(19) United States
(12) Patent Application Publication
(10) Pub. No.: US 2024/0166729 A1
(43) Pub. Date: May 23, 2024

(54) MONOCLONAL ANTIBODY AND USES THEREOF FOR THE TREATMENT AND DIAGNOSIS OF MULTIDRUG-RESISTANT PATHOGENIC BACTERIAL INFECTIONS

(71) Applicants: Ramot at Tel-Aviv University Ltd., Tel Aviv (IL); B.G. Negev Technologies and Applications Ltd., at Ben-Gurion University, Beer Sheva (IL)

(72) Inventors: Yariv WINE, Tel Aviv (IL); Neta SAL-MAN, Lehavim (IL); Yaron HILLMAN, Tel Aviv (IL); Dan LUSTIGER, Tel Aviv (IL); Jenia GERSHBERG, Kiryat Gat (IL); Dan EVEN, Tel Aviv (IL)

(21) Appl. No.: 17/996,013

(22) PCT Filed: Apr. 14, 2021
(86) PCT No.: PCT/IL2021/050430

§ 371 Date: Oct. 12, 2022

Related U.S. Application Data
(60) Provisional application No. 63/010,919, filed on Apr. 16, 2020.
(60) Provisional application No. 63/118,358, filed on Nov. 25, 2020.

(51) Int. Cl. C07K 16/12; A61K 39/00; A61K 45/06; A61P 31/04; G01N 33/569

(52) U.S. Cl. CPC C07K 16/1232; A61K 45/06; A61P 31/04; G01N 33/56916; A61K 2039/505; C07K 2317/33; C07K 2317/34; C07K 2317/565; G01N 2333/245

(57) ABSTRACT

The invention relates to human antibodies specific for Type III secretion system (T3SS), variants and biosimilars thereof. The human antibodies of the invention are specifically directed at the EspB protein. The preset disclosure further provides compositions and uses of the disclosed antibodies in treatment and diagnosis of MDR pathogenic bacterial infections.

The Sequence Listing in ASCII text file format of 31,555 bytes in size, created on Jun. 29, 2023, with the file name “2023-07-14SequenceListing_WINE2A_ST25,” filed in the U.S. Patent and Trademark Office on even date herewith, is hereby incorporated herein by reference.

### FIELD OF THE INVENTION

The present invention generally relates to antibodies specific for multidrug-resistant (MDR) pathogenic bacteria. More specifically, the invention relates to human antibodies specific for Type III secretion system (T3SS), compositions and uses thereof in treatment and diagnosis of MDR pathogenic bacterial infections.

### BACKGROUND ART

References considered to be relevant as background to the presently disclosed subject matter are listed below:
Bhaysar, A. P.; Guttman, J. A.; Finlay, B. B. Nature 449, 827-834 (2007);
Van Bambeke F. et al., (2016) Trends Pharmacol Sci.; 37(9):734-749;
Luz, D. et al., (2017) Front. Immunol. 8:477;
Thanabalasuriar A. et al., (2017) J Clin Invest, 127(6): p. 2249-2261;
WO2017/095744;
Potter A A. et al., (2015) PLoS ONE 10(10): e0139803;
Wang S. et al., (2014) Indian J Microbiol.; 54(2):143-50;
Picking W L. et al., (2017) J Biol Chem.; 292(40):16677-16687;
Jiao X. et al., (2015) Monoclon Antib Immunodiagn Immunother.; 34(6):432-5;
WO13148987;
Baruch, K., et al., (2011) Embo j.; 30(1): p. 221-31;

Acknowledgement of the above references herein is not to be inferred as meaning that these are in any way relevant to the patentability of the presently disclosed subject matter.

### BACKGROUND OF THE INVENTION

The emergence of multiple-drug resistant (MDR) bacterial strains stems largely from the extensive, and sometimes inappropriate, usage of antibiotics in the community and in agriculture, as this misuse has exerted a strong selective pressure on bacteria to develop resistance mechanisms against various antibiotics. In turn, the implications of the increasing numbers of MDR bacterial infections in the clinic, in the community, and in agriculture are constituting a growing global public health concern. MDR bacterial infections are harder to treat and are associated with higher medical costs than antibiotic-sensitive infections, and, perhaps more importantly, there is a significant risk that MDR mechanisms will be spread to other bacterial strains. A parallel public health concern is that the development and approval of new antibiotics has not kept pace with the rising rates of morbidity and mortality due to bacterial infections, giving rise to a predicted annual death rate of 10 million people by 2050 due to resistance to antimicrobials. Pivotal to the efficiency of controlling antibiotic resistance is the ability to provide rapid and accurate surveillance and diagnosis, as is embodied in the WHO One Health concept for addressing the MDR crisis. A particularly promising means for providing such diagnosis lies in monoclonal antibodies (mAbs) targeted against pathogen-specific antigens. mAbs were previously demonstrated as diagnostic agents for the detection of harmful bacteria. In keeping with this line of thought, recent advances in the discovery, engineering, production, and clinical development of mAbs indicate their potential in the design of rapid diagnostics. A major need for rapid diagnosis includes strains of Gram-negative bacterial pathogens, such as Escherichia coli, and species of Salmonella, Shigella, Yersinia, and Pseudomonas, which cause serious diseases, ranging from lethal diarrhea to sepsis, leading to millions of deaths annually. An essential component common to these bacterial pathogens is termed the type 3 secretion system (T3SS). The T3SS is a syringe-like protein complex, which is responsible for injecting virulence factors from the bacterial cytoplasm directly into the human host cell. This T3SS complex is essential for bacterial virulence, as the injected proteins (effectors) manipulate key intracellular host pathways (e.g., cell cycle, immune response, cytoskeletal organization, metabolic processes, and intracellular trafficking) that ultimately promote bacterial replication and transmission [1]. Van Bambeke et al. reviewed several strategies that were used to inhibit T3SS-mediated toxicity. The main families of existing inhibitors comprise small-molecule inhibitors and antibodies that are able to inhibit T3SS expression or function, and to protect host cells from T3SS-mediated cytotoxicity [2]. Luz et al. described the production of a hybrid recombinant EspB toxin that comprises all known variants of this protein found in E. coli strains. Analysis of this recombinant protein allowed to propose immunogenic regions of EspB variants, as well as two common epitopes between known EspB subtypes [3]. Thanabalasuriar et al. have developed a bispecific mAb, targeting two antigens on the surface of Pseudomonas aeruginosa (Psl and PcrV), that was shown to enhance neutrophil uptake of P. aeruginosa and inhibit the T3SS function, thus allowing phagosome acidification and bacterial killing [4]. WO 2017/095744 further discloses a method for preventing or treating nosocomial diseases, e.g., diseases caused by Pseudomonas aeruginosa by administering a specified dose of the bispecific antibody [5]. Potter et al. tested the ability of rabbit polyclonal sera against individual T3SS effector proteins (T3SPs) of Shiga toxin-producing Escherichia coli (STEC) serotype 0103 to block adherence of the organism to HEp-2 cells. It was shown that pooled sera against EspA, EspB, EspF, NleA and Tir lowered the adherence of STEC 0103 relative to pre-immune sera. Pooled anti-STEC 0103 sera were also able to block adherence by STEC 0157 [6]. Wang et al. reported the production of a phage single-chain fragment variable antibody (scFv) library and a specific scFv against the needle protein from the T3SS of Vibrio parahaemolyticus named VP1694 [7]. Picking et al. created a panel of single-V_{H} domain antibodies (VHHs) that recognize distinct epitopes within the Invasion Plasmid Antigen D (IpaD) protein, which is part of the T3SS of Shigella flexneri [8]. Jiao et al. reported the production of two recombinant proteins based on the SpiC protein, a member of Salmonella spp. T3SS. These recombinant proteins were used for generation of monoclonal antibodies which may be useful in the study of SpiC function and for use in the immune diagnosis of Salmonella infection [9]. WO13148987 relates to compositions and methods for treating infection by pathogenic bacteria, specifically by targeting virulence factors of pathogenic E. coli (e.g., intimin) [10]. Instead of the classical antibiotics, antibodies targeting the T3SS may thus represent a potent strategy. Such antibodies have high potential as drugs for preventing and treating infections caused by antibiotic resistant pathogens such as enteropathogenic E. coli (EPEC), enterohemorrhagic E. coli (EHEC) and others. This unmet need should be addressed by developing novel therapeutic drugs against Multiple Drug Resistant (MDR) bacterial strains.

### SUMMARY OF THE INVENTION

In a first aspect, the invention provides an antibody or any antigen-binding fragment thereof, or any matrix, nano- or micro-particles thereof. In some embodiments, the antibody may comprise at least one heavy chain complementarity determining region (CDRH) 1 comprising the amino acid sequence GFTFSHYA, as denoted by SEQ ID NO: 6, or any homologs or derivatives thereof, CDRH2 comprising the amino acid sequence INSNGDST, as denoted by SEQ ID NO: 10, or any homologs or derivatives thereof, CDRH3 comprising the amino acid sequence ARDRRAGYFDYW, as denoted by SEQ ID NO: 14, or any homologs or derivatives thereof, and at least one light chain complementarity determining region (CDRL) 1 comprising the amino acid sequence RDNIGKNY as denoted by SEQ ID NO: 22, or any homologs or derivatives thereof, CDRL2 comprising the amino acid sequence RNN as denoted by SEQ ID NO: 26, or any homologs or derivatives thereof, and CDRL3 comprising the amino acid sequence SAWDTSLNA as denoted by SEQ ID NO: 30, or any homologs or any derivative, variant and biosimilar thereof. In some embodiments, the antibody of the present disclosure is a human antibody. A further aspect of the invention provides a pharmaceutical composition comprising as an active ingredient at least one antibody or any antigen-binding fragment thereof, or any matrix, nano- or micro-particles thereof, in accordance with the invention. In yet another aspect, the invention provides an isolated nucleic acid molecule comprising at least one nucleotide sequence encoding the antibody of the invention or any antigen-binding fragment thereof. Still further, the invention provides vectors comprising the nucleic acid sequences of the invention, as well as host cells expressing these expression vectors. In yet another aspect, the invention relates to a method of treating, preventing, ameliorating, reducing or delaying the onset of an infection by at least one bacterium expressing at least one T3SS in a subject in need thereof. More specifically, the method of the invention comprises the step of administering to the subject a therapeutically effective amount of at least one antibody or any antigen-binding fragment thereof, any matrix, nano- or micro-particles thereof, or of any composition comprising the antibody of the invention. More specifically, the antibody of the invention comprises at least one heavy chain CDRH1 comprising the amino acid sequence GFTFSHYA, as denoted by SEQ ID NO: 6, or any homologs or derivatives thereof, CDRH2 comprising the amino acid sequence INSNGDST, as denoted by SEQ ID NO: 10, or any homologs or derivatives thereof, CDRH3 comprising the amino acid sequence ARDRRAGYFDYW, as denoted by SEQ ID NO: 14, or any homologs or derivatives thereof, and at least one light chain CDRL1 comprising the amino acid sequence RDNIGKNY as denoted by SEQ ID NO: 22, or any homologs or derivatives thereof, CDRL2 comprising the amino acid sequence RNN as denoted by SEQ ID NO: 26, or any homologs or derivatives thereof, and CDRL3 comprising the amino acid sequence SAWDTSLNA as denoted by SEQ ID NO: 30, or any homologs, any derivative, variant and biosimilar of the antibodies of the invention. In a further aspect, the invention relates to a method of treating, preventing, ameliorating, reducing, or delaying the onset of an infection by at least one bacterium expressing at least one T3SS in a subject in need. The method comprising the step of administering to a subject that is treated with at least one anti-bacterial agent, a therapeutically effective amount of at least one isolated antibody of the invention or any antigen-binding fragment thereof, or any composition comprising the antibody. In a further aspect, the invention provides a kit comprising: in a first element (a), at least one antibody or any antigen-binding fragment thereof, or any matrix, nano- or micro-particles thereof, wherein the antibody comprises at least one heavy chain CDRH1 comprising the amino acid sequence GFTFSHYA, as denoted by SEQ ID NO: 6, or any homologs or derivatives thereof, CDRH2 comprising the amino acid sequence INSNGDST, as denoted by SEQ ID NO: 10, or any homologs or derivatives thereof, CDRH3 comprising the amino acid sequence ARDRRAGYFDYW, as denoted by SEQ ID NO: 14, or any homologs or derivatives thereof, and at least one light chain CDRL1 comprising the amino acid sequence RDNIGKNY as denoted by SEQ ID NO: 22, or any homologs or derivatives thereof, CDRL2 comprising the amino acid sequence RNN as denoted by SEQ ID NO: 26, or any homologs or derivatives thereof, and CDRL3 comprising the amino acid sequence SAWDTSLNA as denoted by SEQ ID NO: 30, or any homologs, derivative, variant and biosimilar of the antibodies of the invention. In a second element, (b), the kit of the invention further comprises at least one therapeutic agent, optionally, at least one anti-bacterial agent. In yet another aspect, the invention provides a diagnostic method for detecting at least one bacterium expressing a T3SS in at least one biological sample, the method comprising: the first step (a), involves contacting the at least one biological sample with at least one antibody or any antigen-binding fragment thereof. More specifically, the antibody used by the diagnostic methods of the invention comprises at least one heavy chain CDRH1 comprising the amino acid sequence GFTFSHYA, as denoted by SEQ ID NO: 6, or any homologs or derivatives thereof, CDRH2 comprising the amino acid sequence INSNGDST, as denoted by SEQ ID NO: 10, or any homologs or derivatives thereof, CDRH3 comprising the amino acid sequence ARDRRAGYFDYW, as denoted by SEQ ID NO: 14, or any homologs or derivatives thereof, and at least one light chain CDRL1 comprising the amino acid sequence RDNIGKNY as denoted by SEQ ID NO: 22, or any homologs or derivatives thereof, CDRL2 comprising the amino acid sequence RNN as denoted by SEQ ID NO: 26, or any homologs or derivatives thereof, and CDRL3 comprising the amino acid sequence SAWDTSLNA as denoted by SEQ ID NO: 30, or any homologs or derivatives thereof, or any derivative, variant and biosimilar thereof, wherein the antibody is directly or indirectly associated with at least one detectable moiety. The second step (b), involves determining the presence of the detectable moiety. It should be noted that the presence of the detectable moiety indicates the presence of an at least one bacterium expressing a T3SS in said biological sample. In yet another aspect, the invention provides a diagnostic kit comprising: (a), at least one antibody or any antigen-binding fragment thereof, OR any matrix, nano- or micro-particles thereof, the antibody comprising at least one heavy chain CDRH1 comprising the amino acid sequence GFTFSHYA, as denoted by SEQ ID NO: 6, CDRH2 comprising the amino acid sequence INSNGDST, as denoted by SEQ ID NO: 10, CDRH3 comprising the amino acid sequence ARDRRAGYFDYW, as denoted by SEQ ID NO: 14, and At least one light chain CDRL1 comprising the amino acid sequence RDNIGKNY as denoted by SEQ ID NO: 22, CDRL2 comprising the amino acid sequence RNN as denoted by SEQ ID NO: 26, and CDRL3 comprising the amino acid sequence SAWDTSLNA as denoted by SEQ ID NO: 30, or any derivative, variant and biosimilar thereof, wherein the antibody is directly or indirectly associated with at least one detectable moiety. The kit optionally further comprises at least one of: (b) means for detecting the presence of the at least one detectable moiety; (c) at least one control sample and/or control standard value; (d) instructions for use of the kit. A further aspect of the invention provides a method for modulating the immune system of a subject infected by at least one T3SS expressing bacteria. The method comprising the step of administering to the subject a therapeutically effective amount of at least one antibody in accordance with the invention or of any antigen-binding fragment thereof. These and other aspects of the invention ill become apparent by the hand of the following description.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

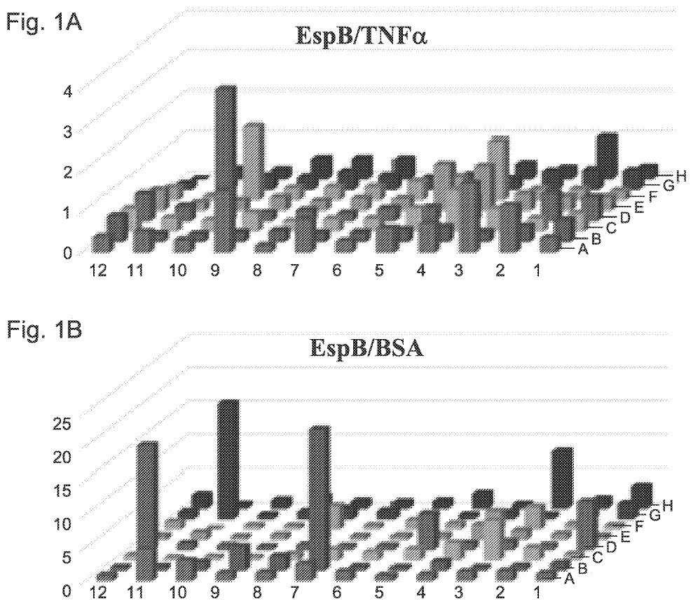

### FIG. 1A-1B: Bio-Panning Cycles Output were Screened by Phage-ELISA

FIG. 1A: Phages from the 3rd cycle output were tested against EspB and human TNF-α.

FIG. 1B: Phages from the 4th cycle output were tested against EspB and BSA.

Three dimensional histograms represent the differential ELISA binding signal of each clone to EspB-control antigen.

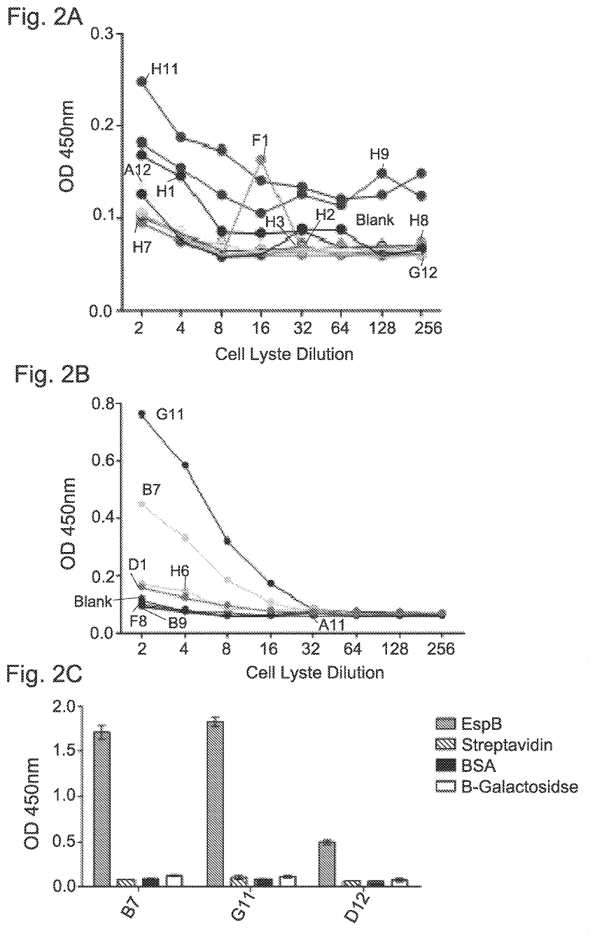

### FIG. 2A-2C: Binding Affinity and Specificity to EspB of scFv Clones by scFv-Soluble ELISA

FIG. 2A: scFv clones from the 3 rd bio-panning cycle were tested against EspB and detected by rabbit anti-CBD tag, and anti-rabbit HRP conjugate antibody.

FIG. 2B: scFv clones from the 4th bio-panning cycle were tested against EspB and two control antigens and detected by rabbit anti-CBD tag, and anti-rabbit HRP conjugate antibody.

FIG. 2C: Three scFv's clones (B7, G11, D12) were tested for binding specificity to EspB compared to two control antigens by scFv-soluble ELISA.

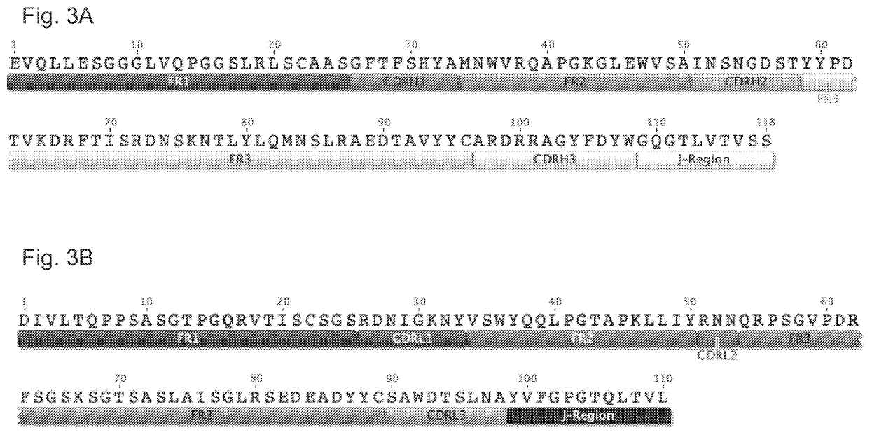

### FIG. 3A-3B: Schematic Representation of the Sequences of the VH and VL of the mAb-B7 Antibody

FIG. 3A: Schematic representation of the amino acid sequence of the V_{H}, as denoted by SEQ ID NO: 2 (encoded by nucleic acid sequence of SEQ ID NO: 1) as well as its 3 CDRs (as denoted by SEQ ID NO: 6, 10, 14).

FIG. 3B: Schematic representation of the amino acid sequence of the V_{L}, as denoted by SEQ ID NO: 18 (encoded by nucleic acid sequence of SEQ ID NO: 17) as well as its 3 CDRs (SEQ ID NO: 22, 26, 30).

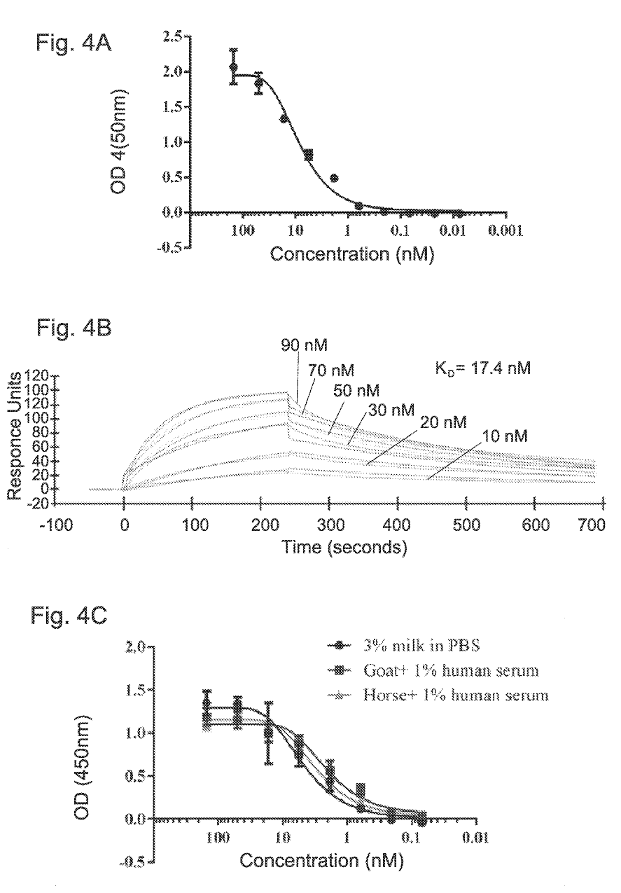

### FIG. 4A-4C: mAb-B7 Binds to EspB with a Nanomolar Affinity

FIG. 4A: Graph showing results of ELISA binding assay of mAb-B7 by coating with 5 μg/ml of EspB or no antigen for control. Error bars represent+/−SD.

FIG. 4B: SPR sensorgrams of mAb-EspB-B7 binding to an EspB-coated chip. mAb-EspB-B7 was added at various concentrations between 10 and 90 nM. Sensorgrams were fitted to the steady-state model.

FIG. 4C: Graph showing results of ELISA binding affinity of mAb-B7 to EspB in 1% human serum or in 3% skim milk in PBS for control. Error bars represent+/−SD.

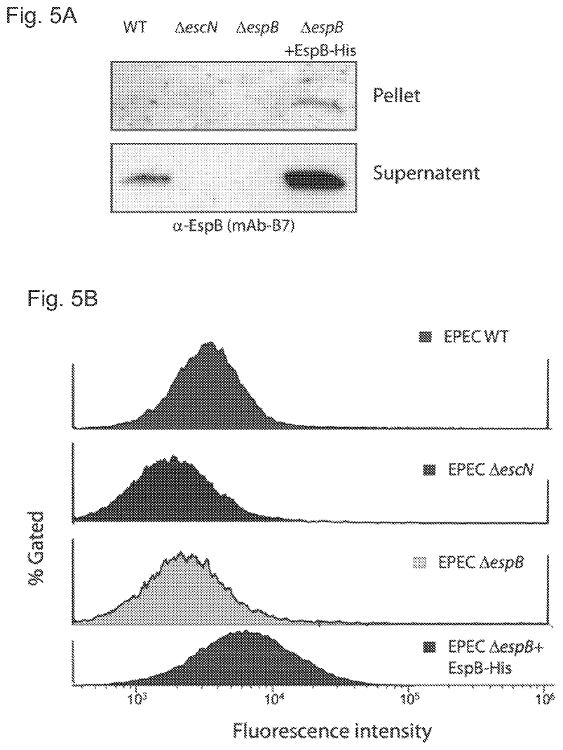

### FIG. 5A-5B: mAb-EspB-B7 Binds to Recombinant and Native EspB

FIG. 5A. EPEC wild type (WT), AescN, AespB and AespB expressing EspB-His strains were grown under T3SS-inducing conditions for 6 hr. The bacterial pellets and supernatants were separated and analyzed using SDS-PAGE and western blotting with mAb-EspB-B7. EspB expression within the bacteria (pellet) was observed only for the AespB+EspB-His strain, while EspB secretion (supernatant) was observed for both WT EPEC and the complemented AespB+EspB-His strain.

FIG. 5B. EPEC WT, AescN, AespB and AespB+EspB-His bacteria were grown under T3SS-inducing conditions for 3 hr. Thereafter, 1×10⁷ bacteria were incubated with mAb-EspB-B7, washed, and stained with Alexa Fluor 488 goat anti-human IgG antibody. Flow cytometry analysis was performed on a Gallios instrument (Beckman coulter).

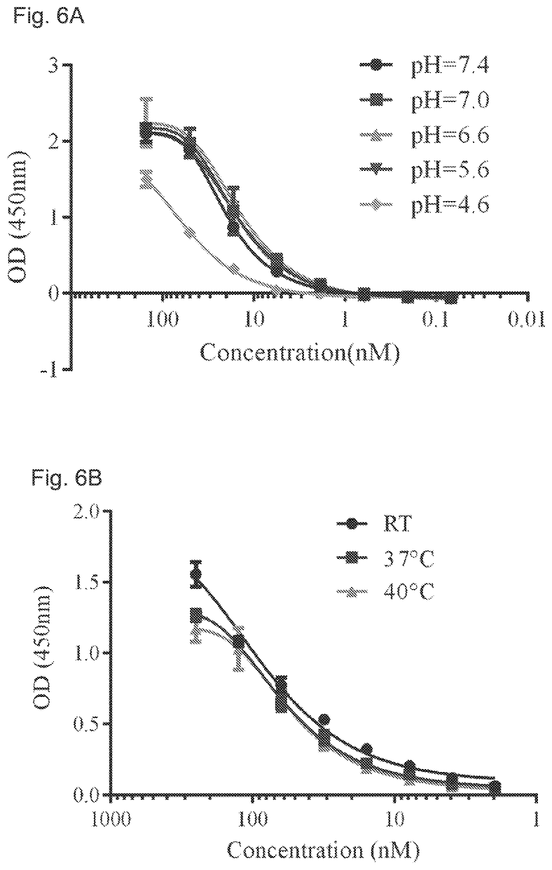

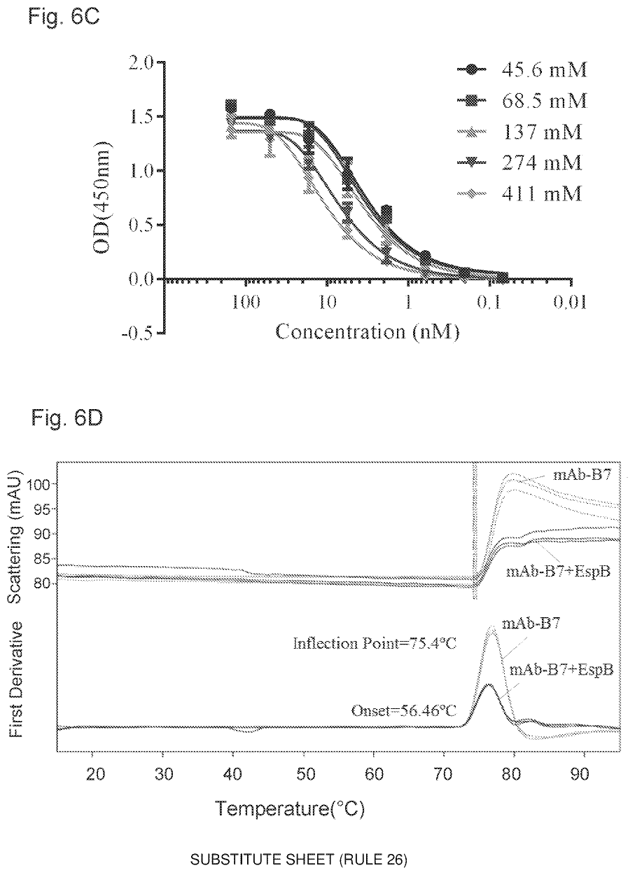

### FIG. 6A-6D: Stability of mAb-B7

FIG. 6A: ELISA assay for measuring mAb-B7 binding affinity to EspB in different pH conditions (4.6-7.4) at temperatures of 37° C., 40° C., or room temperature (RT). Error bars represent+/−SD.

FIG. 6B: ELISA assay for measuring mAb-B7 binding affinity to EspB at temperatures of 37° C., 40° C., or room temperature (RT). Error bars represent+/−SD.

FIG. 6C: ELISA assay for measuring mAb-B7 binding affinity to EspB in various NaCl concentrations (45.6-411 mM). Error bars represent+/−SD.

FIG. 6D: Evaluation of mAb-B7 melting temperature (Tm) by nanoDSF, Prometheus NT.48, NanoTemper.

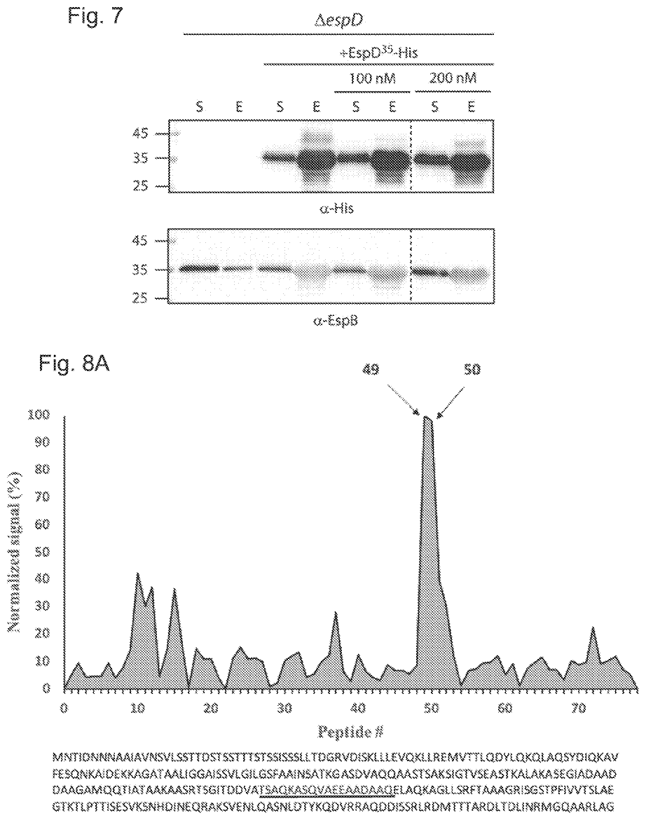

### FIG. 7. mAb-EspB-B7 does not Interfere with the EspB-EspD Interaction

Supernatants of EPEC ΔespD expressing EspD-³⁵His were purified using Ni-NTA beads. EPEC ΔespD strain without the pEspD-³⁵Hs expression vector, was used as a negative control. Samples of supernatants (S) and elution (E) fractions were loaded on SDS-PAGE and analyzed by western blotting with mouse anti-His and anti-EspB antibodies (to avoid detection of the human EspB antibody). Analysis of the supernatants confirmed EspB and EspD secretion into the extracellular medium. The co-elution of EspB with EspD-³⁵His was not affected by the absence or the presence (100 nM and 200 nM) of mAb-EspB-B7. Low EspB non-specific binding to the Ni-NTA beads was detected (in the absence of EspD-³⁵His).

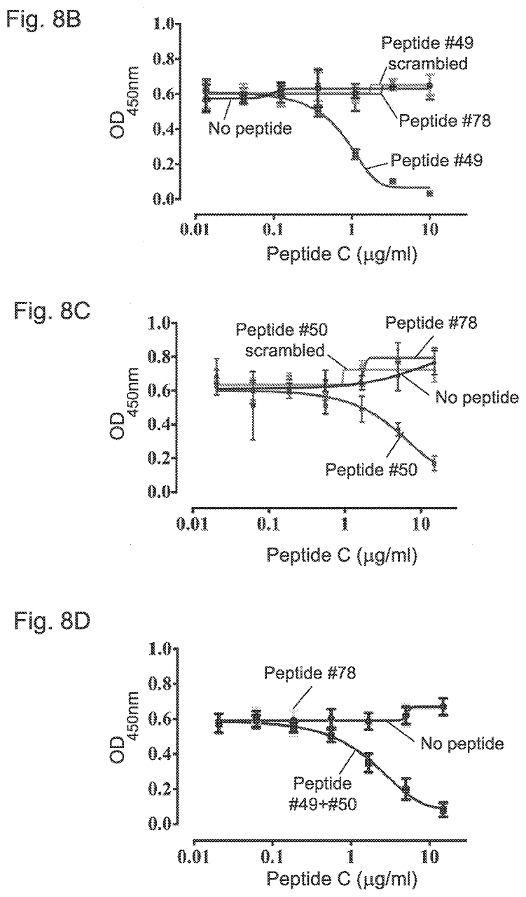

### FIG. 8A-8D. mAb-EspB-B7 Epitope Mapping

FIG. 8A. An EspB pepstar peptide array of 78 cyclic peptides (15-residue long peptides with an 11-residue overlap) was examined for mAb-EspB-B7 binding. Image analysis was carried out with Genepix Pro 6.0 analysis software (Molecular Devices) to detect antibody binding; fluorescence signals were normalized showing their relative intensities. The putative binding site of mAb-EspB-B7 along the EspB protein is marked in underlined bold. Arrows indicate the signals obtained from peptides #49 and #50, which displayed the highest signal intensities. The EspB amino acid sequence in the figure is denoted by SEQ ID NO: 40.

FIGS. 8B, 8C, 8D. mAb-EspB-B7 binding to EspB following pre-incubation with peptide #49 and peptide #49 scrambled (FIG. 8B), peptide #50 and peptide #50 scrambled (FIG. 8C), or peptide #49+50 (FIG. 8D) was evaluated by competitive ELISA and detected using anti-human IgG HRP-conjugated antibody. Peptide #78 was used as a negative control. Error bars represent ±SD.

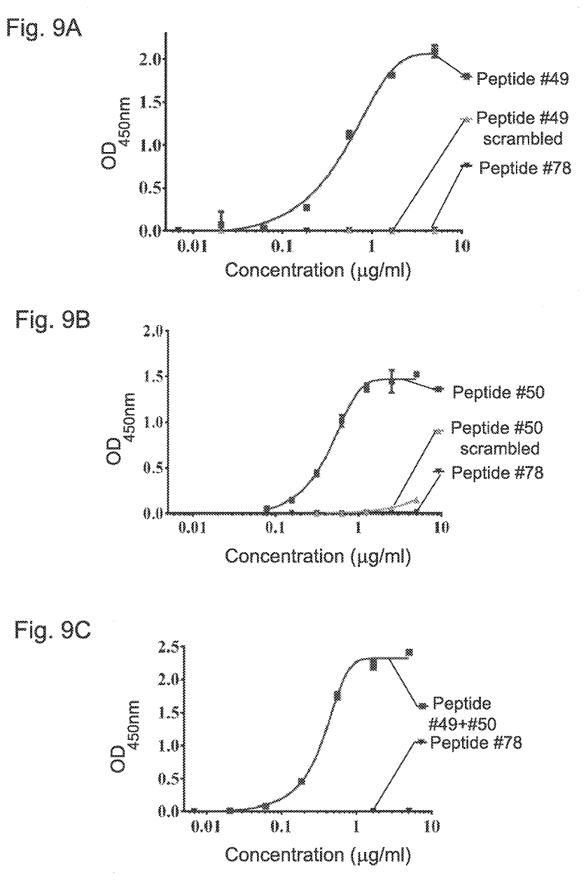

### FIG. 9A-9C. mAb-EspB-B7 Binding to EspB Peptides

FIGS. 9A, 9B, 9C, show mAb-EspB-B7 binding to peptide #49 and peptide #49 scrambled (SEQ ID NO: 33 and 34, respectively) (FIG. 9A), peptide #50 and peptide #50 scrambled (SEQ ID NO: 35 and 36, respectively) (FIG. 9B), or to peptide #49+50 (SEQ ID NO: 37) (FIG. 9C) was evaluated by ELISA. A 96 well plate was coated with the peptides before being incubated with serially diluted mAb-B7 and detected using anti-human IgG HRP-conjugated antibody. Peptide #78 (SEQ ID NO: 38) was used as a negative control. Error bars represent+/−SD. Each peptide was synthesized with the addition of cysteine residues at the C and N-termini, to enable peptide cyclization.

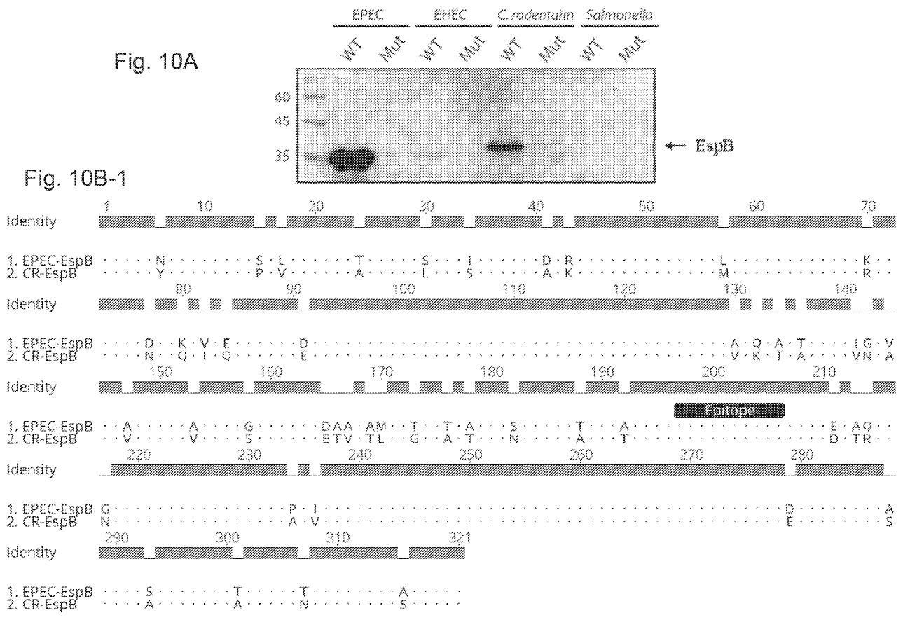

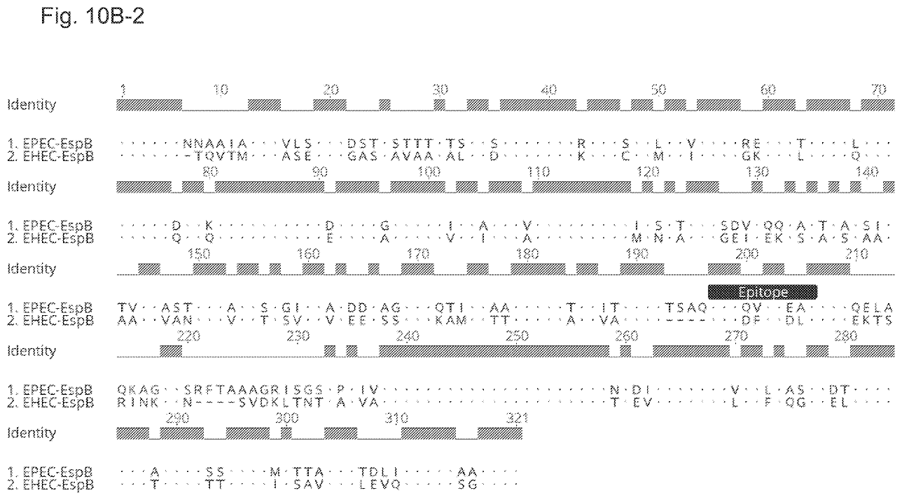

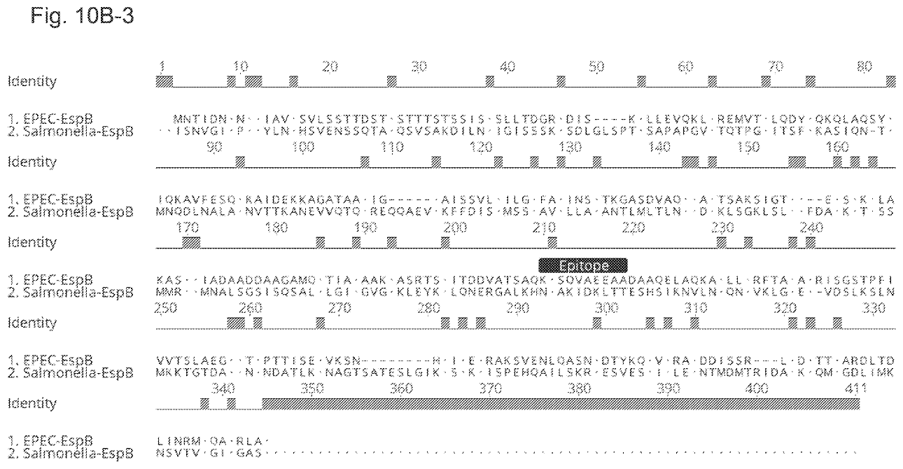

### FIG. 10A-10B. mAb-EspB-B7 Binds EspB Homologs in Other T3SS-Expressing Bacteria

FIG. 10A. Wild type and mutant EPEC, EHEC, C. rodentium and Salmonella were grown under T3SS-inducing conditions. EPEC, EHEC and C. rodentium mutant strains contain a deletion in the escN gene, while Salmonella contains a deletion in the invA gene, which results in non-functional T3SSs in these mutant strains. The bacterial cultures were centrifuged, and the supernatants were collected, normalized, and analyzed by SDS-PAGE and western blotting using mAb-EspB-B7.

FIG. 10B-1-10B-3. Amino acid sequence alignment of EspB from EPEC (SEQ ID NO: 40) with C. rodentium (CR, SEQ ID NO: 46, 10B-1), EHEC (SEQ ID NO: 45, 10B-2), or Salmonella (SEQ ID NO: 47, 10B-3) EspB homologs. The dark bars and/or dots represent identical amino acids in each corresponding sequence, the different residues are indicated. The mAb-EspB-B7 epitope is annotated above the amino acids that are part of the epitope.

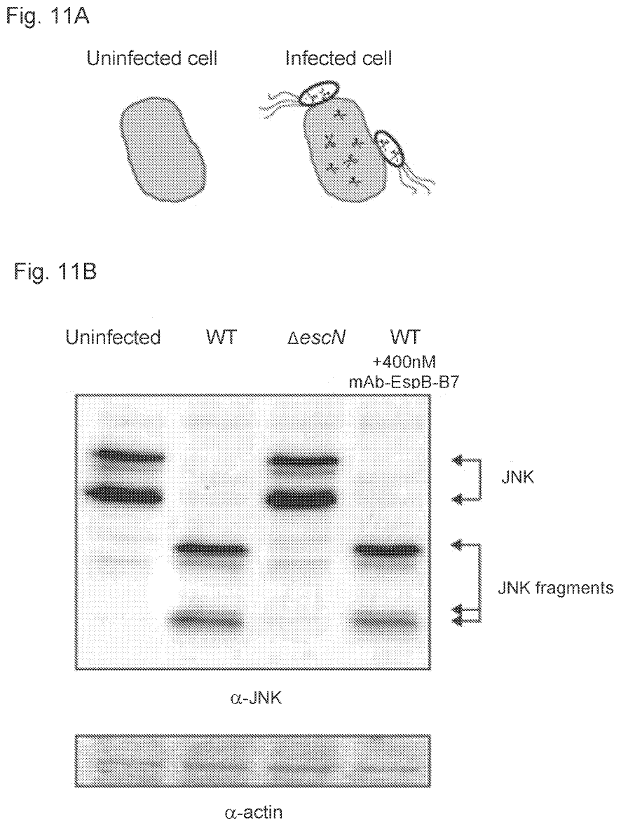

### FIG. 11A-11B. mAb-EspB-B7 does not Inhibit EPEC Translocation Activity into HeLa Cells

FIG. 11A. Scheme of the effector translocation assay. Infection of HeLa cells with EPEC was monitored by detecting the degradation profile of JNK, a human kinase that is subjected to cleavage by the EPEC effector, NleD.

FIG. 11B. HeLa cells were infected with wild-type (WT) EPEC in the presence or absence of 400 nM mAb-EspB-B7. After 3 hr, cells were washed, and host cell proteins were extracted and subjected to western blot analysis using anti-JNK and anti-actin (loading control) antibodies. JNK and its degradation fragments are indicated at the right of the gel. Degradation of JNK was evident in the WT EPEC, sample but not in the uninfected sample or in the samples infected with EPEC AescN. HeLa cells infected with WT EPEC in the presence of 400 nM mAb-EspB-B7 showed a JNK degradation profile similar to that of WT EPEC in the absence of mAb-EspB-B7.

### DETAILED DESCRIPTION OF THE INVENTION

Before specific aspects and embodiments of the invention are described in detail, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims. The problem of antibiotic-resistant pathogens represents a critical unmet need for public health. Instead of the classical antibiotics used so far in the clinic, the inventors have focused their efforts on a biological drug that should overcome the challenge of developing a new antibiotic. As shown in the Examples, a monoclonal antibody (named mAb-B7) was isolated which targets specifically food-borne pathogenic bacteria such as enteropathogenic E. coli (EPEC) that utilize the T3SS for their virulence. Specifically, and as demonstrated by the Examples, the monoclonal antibody developed herein targets EspB, an essential component of the T3SS. This enables inhibiting the ability of the bacteria to inject virulence proteins to the host cell through the T3SS, thus inhibiting bacterial survival. This specific antibody, that is a human antibody made of human sequences, was sequenced and characterized as disclosed by EXAMPLE 1. As shown in EXAMPLE 2, the mAb-B7 was characterized and found to bind EPEC EspB with nM affinity, as determined by Surface Plasmon Resonance (SPR). The mAb-B7 was shown to bind to recombinant and wild-type EspB both in its free and membrane-anchored forms. The mAb-B7 was also subjected to a series of stability assays and found to retain its stability at various pH levels, temperatures and NaCl concentrations, and its thermal stability was confirmed by nano Differential Scanning Fluorimetry (nanoDSF, see EXAMPLE 4). Moreover, the binding site between mAb-B7 and EspB was determined by epitope mapping and validated by ELISA (EXAMPLE 6). Identical sequences of mAb-B7 binding sites were identified in both EPEC EspB and C. rodentium EspB, a related murine pathogen that is commonly used in in vivo experiments of bacterial diarrheal diseases, as shown in EXAMPLE 7. The mAb-B7 was found to bind C. rodentium EspB as well as to EPEC-related bacteria, Enterohemorrhagic E. coli (EHEC), thus suggesting the potential of mAb-B7 as an inhibitor of pathogenicity in vivo. More specifically, the present disclosure describes a mAb raised against EspB, an essential component within the T3SS that is crucial for the infectivity of numerous Gram-negative bacteria, including EPEC. The results disclosed herein demonstrate that mAb-EspB-B7 binds EspB with nM affinity and high specificity. As commercial monoclonal antibodies against bacterial species, targeted mostly against common bacterial antigen such as the flagella or the bacterial Lipopolysaccharides (LPS), have been reported to have micromolar affinities, the mAb-EspB-B7 holds greater potential to allow efficient detection of bacterial pathogens due to its nM affinity. The antibody binding to its EspB target was stable over a wide range of pH values, excluding acidic pH values, and across various salt concentrations. A reduced binding capacity was detected only under high salt concentrations (>250 mM), suggesting that the antibody-antigen binding interface is governed by electrostatic interactions. This idea is supported by the observation that the identified EspB epitope contains nearly 50% of charged amino acids, which might be involved in the antibody-antigen binding. mAb-EspB-B7 demonstrated a relatively high melting temperature, which was moderately elevated when the antibody was complexed with its antigen. This result suggests that EspB binding has a stabilizing effect on the antibody, as was previously reported for anti-ricin neutralizing antibody. Furthermore, the melting temperature profile of mAb-EspB-B7 showed three distinct events that probably correspond to the melting order of the C_{H}2 region, followed by the Fab and C_{H}3, as reported previously. This melting profile indicates that the mAb-EspB-B7 would be suitable for applications that require relatively high thermal stability. The rational for pinpointing EspB derived from the fact that EspB is getting exposed to the extracellular environment following EPEC entrance to the digestive system and in response to thermal and chemical signals. Based on the number of T3SS complexes expressed on each bacterium and the predicted number of EspB subunits found in each T3SS complex, the inventors estimated that there are approximately 100 EspB molecules per each bacterial cell. The present disclosure reports the development and characterization of mAb-EspB-B7 and further demonstrate its potential as a bio-recognition element. The mAb-EspB-B7 demonstrated high specificity and affinity towards EspB, binding capacity to soluble EspB and in the context of whole bacteria, and high stability under a variety of conditions. Epitope mapping using the specially designed cyclic-peptide array revealed that mAb-EspB-B7 binds mostly to a specific amino acid sequence located at positions 193-210 along the EspB sequence (SEQ ID NO: 39). In a previous study, it was shown that this region was not important for EspB-EspD interactions, a fact that was further corroborated by our observation that mAb-EspB-B7 does not disrupt the interaction between the two proteins. Moreover, the observation that mAb-EspB-B7 binds EspB as a component of the fully assembled T3SS complex supports the notion that the epitope of EspB is exposed and not buried within the EspB-EspD interface. It is noteworthy that the peptide array results also identified an additional region, corresponding to peptides #9-12 (SEQ ID NO: 48), that demonstrated mAb-EspB-B7 binding. This finding could perhaps suggest that the epitope recognized by mAb-EspB-B7 is conformational rather than linear. As the main epitope sequence (positions 193-210, (SEQ ID NO: 39)) is fully conserved in EPEC and C. rodentium, the lower similarity along this second region might provide an explanation for the reduced western blot signal that was observed for C. rodentium EspB (FIG. 10A). In addition, while mAb-EspB-B7 binding to a protein was observed in the supernatants of WT EHEC and C. rodentium, no binding was detected in the Salmonella supernatant. This result is in agreement with the presence of the epitope in EHEC and C. rodentium but not in Salmonella (FIG. 10B). The ability of mAb-EspB-B7 to recognize and bind C. rodentium EspB is highly important, as it provides the scientific grounds for the use of the mAb-EspB-B7 antibody as diagnosis tool of mice infection model. In addition, while mAb-EspB-B7 did not demonstrate a reduction of bacterial infectivity in the ex vivo system, the inventors posit that examining it in a mouse model will provide a more comprehensive picture that will include the effect of the antibody in promoting certain activities of the immune system against bacteria, such as opsonization and phagocytic clearance. These activities may prevent the spread of the bacterial infection within the host body and induce a humoral response with serological memory that will shorten the infection duration, promote recovery and provide cellular and serological memory. Another key aspect of mAb-EspB-B7 is its ability to bind both the secreted form of EspB and EspB as a component of the assembled T3SS complex within the bacterial cell. This finding provides further support for its potential as a diagnostic agent capable of detecting bacterial infections directly in clinical samples in a short time with high accuracy, as previously reported. Therefore, in a first aspect, the invention provides an antibody or any antigen-binding fragment thereof. In some embodiments, the antibody may comprise at least one heavy chain complementarity determining region (CDRH) 1 comprising the amino acid sequence GFTFSHYA, as denoted by SEQ ID NO: 6, or any homologs or derivatives thereof, CDRH2 comprising the amino acid sequence INSNGDST, as denoted by SEQ ID NO: 10, or any homologs or derivatives thereof, CDRH3 comprising the amino acid sequence ARDRRAGYFDYW, as denoted by SEQ ID NO: 14, or any homologs or derivatives thereof, and at least one light chain complementarity determining region (CDRL) 1 comprising the amino acid sequence RDNIGKNY as denoted by SEQ ID NO: 22, or any homologs or derivatives thereof, CDRL2 comprising the amino acid sequence RNN as denoted by SEQ ID NO: 26, or any homologs or derivatives thereof, and CDRL3 comprising the amino acid sequence SAWDTSLNA as denoted by SEQ ID NO: 30, or any homologs, derivative, variant and biosimilar of the antibody of the invention. As indicated in more detail herein after, the invention encompasses the antibody of the invention or any antigen-binding fragment thereof that may include in some embodiments, either the heavy or the light chain thereof. As such, the antigen-binding domain of the antibody of the invention may comprise in some embodiments the HCDR1, HCDR2, HCDR3, that may comprise in some further embodiments, the amino acid sequences of SEQ ID NO: 6, 10 and 14, respectively, or any variants and derivatives thereof. In some alternative embodiments, the antigen-binding domain of the antibody of the invention may comprise the LCDR1, LCDR2, LCDR3, that may comprise in some further embodiments, the amino acid sequences of SEQ ID NO: 22, 26 and 30, respectively, or any variants and derivatives thereof. In yet some further embodiments, the HCDR1, HCDR2, HCDR3, of the antibody of the invention or of any antigen-binding fragment thereof are encoded by the nucleic acid sequences that comprise SEQ ID NO: 5, 9, 13, respectively. In yet some further embodiments, the LCDR1, LCDR2, LCDR3, of the antibody of the invention or of any antigen-binding fragment thereof, are encoded by the nucleic acid sequences that comprise SEQ ID NO: 21, 25, 29, respectively. In some other embodiments, the antibody may comprise at least one heavy chain variable region comprising the amino acid sequence denoted by SEQ ID NO: 2 or any homologs, derivatives, or variants thereof and at least one light chain variable region comprising the amino acid sequence denoted by SEQ ID NO: 18 or any homologs, derivatives or variants thereof. In some embodiments, the Framework Regions FR1, FR2, FR3, of the heavy chain of the antibody of the invention or of any antigen-binding fragment thereof, may be encoded by SEQ ID NO: 3, 7, 11, respectively. In yet some embodiments, the Framework Regions FR1, FR2, FR3, of the light chain of the antibody of the invention or of any antigen-binding fragment thereof may be encoded by SEQ ID NO: 19, 23, 27, respectively. Thus, in more specific embodiments, the isolated monoclonal antibody or any antigen-binding fragment thereof may comprise a Heavy chain Framework Region 1 (FR1) comprising the amino acid sequence denoted by SEQ ID NO: 4, or any homologs or derivatives thereof, a heavy chain FR2 comprising the amino acid sequence denoted by SEQ ID NO: 8, or any homologs or derivatives thereof and a heavy chain FR3 comprising the amino acid sequence denoted by SEQ ID NO: 12, or any homologs or derivatives thereof, and a Light chain Framework Region 1 (FR1) comprising the amino acid sequence denoted by SEQ ID NO: 20, or any homologs or derivatives thereof, a Light chain FR2 comprising the amino acid sequence denoted by SEQ ID NO: 24, or any homologs or derivatives thereof, and a Light chain FR3 comprising the amino acid sequence denoted by SEQ ID NO: 28, or any homologs or derivatives thereof. Still further, in some embodiments, the J-region of the heavy chain of the antibody of the invention or of any antigen-binding ragmen thereof may be encoded by the nucleic acid sequence of SEQ ID NO: 15, or any variant thereof. In yet some further embodiments, the J-region of the heavy chain comprises the amino acid sequence as denoted by SEQ ID NO: 16. In yet some further embodiments, the J-region of the light chain of the antibody of the invention or of any antigen-binding ragmen thereof may be encoded by the nucleic acid sequence of SEQ ID NO: 31, or any variant thereof. In yet some further embodiments, the J-region of the light chain comprises the amino acid sequence as denoted by SEQ ID NO: 32. In some embodiments, the antibody may comprise a heavy chain variable region and a light chain variable region, specifically, comprising CDR sequences as described above. In some specific embodiments, the heavy chain variable region is encoded by a nucleic acid sequence which is at least 70% identical to the nucleic acid sequence denoted by SEQ ID NO: 1, or any homologs or derivatives thereof. In yet some further embodiments, the light chain variable region is encoded by a nucleic acid sequence which is at least 70% identical to SEQ ID NO: 17, or any homologs or derivatives thereof. In some embodiments, at least 70% of identity is meant any one of 75%, 80%, 85%, 90%, 95%, 96%, 97%, 98%, 99%, or even 100% identity to the entire sequence. In some other embodiments, the antibody may comprise a heavy chain variable region comprising an amino acid sequence that is at least 70%, 75%, 80%, 85%, 90%, 95%, 96%, 97%, 98%, 99% or 100% identical to SEQ ID NO: 2 or any homologs, derivatives or variants thereof and a light chain variable region comprising an amino acid sequence that is at least 70%, 75%, 80%, 85%, 90%, 95%, 96%, 97%, 98%, 99% or 100% identical to SEQ ID NO: 18 or any homologs, derivatives or variants thereof. The term “antibody” as used herein, means any antigen-binding molecule or molecular complex that specifically binds to or interacts with a particular antigen of any fragments thereof. The term “antibody” includes immunoglobulin molecules typically comprising four polypeptide chains, two heavy (H) chains and two light (L) chains inter-connected by disulfide bonds, as well as dimers (e.g., IgA) and multimers thereof (e.g., IgM). Each heavy chain comprises a heavy chain variable region (abbreviated herein as HCVR or V_{H}) and a heavy chain constant region (CH). The heavy chain constant region comprises three domains, CH1, CH2 and CH3. Each light chain comprises a light chain variable region (abbreviated herein as LCVR or V_{L}) and a light chain constant region. The light chain constant region comprises one domain (CL). The V_{H} and V_{L} regions can be further subdivided into regions of hypervariability, termed complementarity determining regions (CDRs), interspersed with regions that are more conserved, termed framework regions (FR). Each V H and V_{L} is composed of three CDRs and four FRs, arranged from amino-terminus to carboxy-terminus in the following order: FR1, CDR1, PR2, CDR2, FR3, CDR3, FR4. Typically, an antibody is composed of two immunoglobulin (Ig) heavy chains and two Ig light chains. In humans, antibodies are encoded by three independent gene loci, namely kappa (κ) chain (Igκ) and lambda (λ) chain (Igλ) genes for the Light chains and IgH genes for the heavy chains, which are located on chromosome 2, chromosome 22, and chromosome 14, respectively. Still further, it should be appreciated that the antibody of the invention may be of any isotype, for example, IgG1, IgG2, IgG3, IgG4, IgM, IgA1, IgA2, IgE, IgD. The antibody of the invention is in some embodiments a monoclonal antibody. In yet some further embodiments, the antibody of the invention was isolated from a human library and as such is a human antibody, or at least part thereof is of a human origin. The term “human antibody”, as used herein, typically refers to an antibody comprising at least one chain in which the complementarity determining regions (CDR) of the variable domains are human, or synthetic, and whereas the remainder of the chain is of human origin. In some embodiments, the human antibody may be a result humanization, where the variable domain of a humanized chain has a V region amino acid sequence which, analyzed as a whole, is closer to human than to other species, where identity to human sequences must be ≤85%, specifically, 85%, 90%, 91%, 92%, 93%, 94%, 95%, 96%, 96%, 97%, 98%, 99%, or 100%. It should be noted that the human antibodies of the invention may be made by any process of generating fully human mAb, including phage display technology, synthetic libraries, direct, isolation from humans, transgenic animals, humanization techniques, and the like. Still further, the invention further encompasses any chimeric antibody comprising any of the V_{H}, V_{L}, or any of the CDRs disclosed herein. A chimeric antibody as used herein, is one for which both chain types are chimeric as a result of antibody engineering. A chimeric chain is a chain that contains a foreign variable domain (originating from one species other than human, or synthetic or engineered from any species including human) linked to a constant region of human origin. The variable domain of a chimeric chain has a V region amino acid sequence which, analyzed as a whole, is closer to non-human species than to human. In some embodiments, the antibody of the invention is a monoclonal antibody. A monoclonal antibody, as used herein refers to an antibody produced by a single clone of cells or cell line producing identical antibody molecules. Monoclonal antibodies in the form of IgG display bi-valent affinity in binding the same epitope. As indicted above, it should be further understood that the present invention further encompasses any functional fragments of the antibody of the invention, such fragments are referred to herein as antigen binding fragments. The term “an antigen-binding fragment” refers to any portion of an antibody that retains binding to the antigen. Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR)). Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment,” as used herein. Examples of antibody functional fragments include but are not limited to a single-domain antibody (sdAb) which refers to an antibody fragment consisting of a single monomeric variable antibody domain. The first single-domain antibodies were engineered from heavy-chain antibodies found in camelids; these are called VHH fragments. Cartilaginous fishes also have heavy-chain antibodies (IgNAR, ‘immunoglobulin new antigen receptor’), from which single-domain antibodies called variable new antigen receptor antibody (V-NAR) fragments can be obtained. A further example is binding-domain immunoglobulin fusion proteins comprising (i) a binding domain polypeptide that is fused to an immunoglobulin hinge region polypeptide, (ii) an immunoglobulin heavy chain CH2 constant region fused to the hinge region, and (iii) an immunoglobulin heavy chain CH3 constant region fused to the CH2 constant region. The binding domain polypeptide can be a heavy chain variable region or a light chain variable region. These antibody fragments are obtained using conventional techniques known to those with skill in the art, and the fragments are screened for utility in the same manner as are intact antibodies. Still further, in some embodiments, the antibodies disclosed by the invention may be camelid single-domain antibodies (sdAbs), also known as heavy chain-only antibodies (HCAbs) or V_{H}Hs, and widely known as nanobodies. An alternative approach for creating an antigen binding domain in accordance with the invention, is to split the dimeric variable domains from common immunoglobulin G (IgG) from humans or mice into monomers. Although most research into single-domain antibodies is currently based on heavy chain variable domains, nanobodies derived from light chains have also been shown to bind specifically to target epitopes. Thus, it should be further appreciated that in some embodiments, the invention further encompasses a polypeptide comprising a variable region of a light chain comprising at least one of the CDR sequences as denoted by SEQ ID NO: 22, 26 and 30, or any variants, homologs or derivatives thereof. In yet some further embodiments, the polypeptide of the invention may comprise the sequence of a variable region, as denoted by SEQ ID NO: 18. In yet some further embodiments, the invention further provides a polypeptide comprising a variable region of an antibody heavy chain. In some specific embodiments, such polypeptide may comprise the amino acid sequence of at least one of the following CDRs, specifically, of SEQ ID NO: 6, 10 and 14, or any variants homologs or derivatives thereof. In yet some further embodiments, the polypeptide of the invention may comprise the variable region of the heavy chain as denoted by SEQ ID NO: 2, or any homologs or derivatives thereof. As appreciated by one of skill in the art, various antibody fragments can be obtained by a variety of methods, for example, digestion of an intact antibody with an enzyme, such as pepsin, or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries. The term antibody also includes multivalent antibodies, specifically, bivalent molecules, diabodies, triabodies, tetrabodies and the like. References to “V_{H}” or a “VH” refer to the variable region of an immunoglobulin heavy chain, including an Fv, scFv, a disulfilde-stabilized Fv (dsFv) or Fab. References to “V_{L}” or a “VL” refer to the variable region of an immunoglobulin light chain, including of an Fv, scFv, dsFv or Fab. More specifically, the phrase “single chain Fv” or “scFv” refers to an antibody in which the variable domains of the heavy chain and of the light chain of a traditional two chain antibody have been joined to form one chain. Typically, a linker peptide is inserted between the two chains to allow for the stabilization of the variable domains without interfering with the proper folding and creation of an active binding site. A single chain antibody applicable for the invention, e.g., may bind as a monomer. Other exemplary single chain antibodies may form diabodies, triabodies, and tetrabodies. It should be appreciated that in some embodiments, any antibody provided by the invention and used by the methods, and kits of the invention is not a naturally occurring antibody. Specifically, any of the antibodies used herein cannot be considered as a product of nature. In yet some further embodiments, it should be noted that the epitope recognized by the antibodies of the invention may comprise, at least part of residues 185 to 250, specifically, residues 190 to 215, more specifically, 193 to 210 of the EspB protein, specifically, the EspB as denoted by SEQ ID NO: 40. Still further, in some embodiments, the antibody of the invention comprises at least part of the amino acid sequence TSAQKASQVAEEAADAAQ, as denoted by SEQ ID NO: 39. According to Donnenberg et al. (Donnenberg et al. (2011) Journal of Bacteriology; p2972-2980), the EspB protein adopts a transmembrane topology with its C-terminus facing the host cytoplasm. Therefore, the epitope should be found inside the host cell following bacterial infection. It should be appreciated that the invention further encompasses in some embodiments thereof any antibody that recognizes and binds an epitope comprising the amino acid sequence as denoted by SEQ ID NO: 39, or any variants, homologs, fragments or derivatives thereof. In some embodiments, the mAb-B7 antibody recognized the epitope as denoted by SEQ ID NO: 49, that is a fragment of SEQ ID NO: 39. Still further, as shown by the array of EXAMPLE 6, the mAb-B7 antibody further recognizes the residues of peptide #9-12, as denoted by SEQ ID NO: 48. Thus, in some embodiments, the present disclosure further encompasses any antibody the recognizes and binds the epitope of at least one of SEQ ID NO; 39, 48 and 49, or any combinations thereof. The term “epitope” is meant to refer to that portion of any molecule capable of being bound by an antibody which can also be recognized by that antibody. Epitopes or “antigenic determinants” usually consist of chemically active surface groupings of molecules such as amino acids or sugar side chains and have specific three-dimensional structural characteristics as well as specific charge characteristics. In yet some further embodiments, the antibody of the invention cannot be considered as naturally occurring antibody. As such, the antibody of the invention is not a product of nature. In yet some further embodiments, the antibody of the invention is made of human sequences and is therefore a human antibody. As such, the antibody of the invention may be used for therapeutic applications in human subjects. The invention relates to antibodies that are polypeptides comprising amino acid sequences. “Amino acid sequence” or “peptide sequence” is the order in which amino acid residues connected by peptide bonds, lie in the chain in peptides and proteins. The sequence is generally reported from the N-terminal end containing free amino group to the C-terminal end containing amide Amino acid sequence is often called peptide, protein sequence if it represents the primary structure of a protein, however one must discern between the terms “Amino acid sequence” or “peptide sequence” and “protein”, since a protein is defined as an amino acid sequence folded into a specific three-dimensional configuration and that had typically undergone post-translational modifications, such as phosphorylation, acetylation, glycosylation, manosylation, amidation, carboxylation, sulfhydryl bond formation, cleavage and the like. It should be appreciated that the invention encompasses the use of any variant or derivative of the antibody of the invention and any antibodies that are substantially identical or homologue to the antibodies encoded by the nucleic acid sequence of the invention. The term “derivative” is used to define amino acid sequences (polypeptide), with any insertions, deletions, substitutions and modifications to the amino acid sequences (polypeptide) that do not alter the activity of the original polypeptides. By the term “derivative” it is also referred to homologues, variants and analogues thereof. Proteins orthologs or homologues having a sequence homology or identity to the proteins of interest in accordance with the invention, specifically antibodies described herein, may share at least 50%, at least 60% and specifically 65%, 70%, 75%, 80%, 85%, 90%, 91%, 92%, 93%, 94%, 95%, 96%, 97%, 98%, 99%, or higher, specifically as compared to the entire sequence of the proteins of interest in accordance with the invention, for example, any of the antibodies that comprise the amino acid sequence as denoted by any one of SEQ ID NO: 2 and 18, or any one of the CDRs of SEQ ID NO: 6, 10, 14, 22, 26 and 30. Specifically, homologs that comprise or consists of an amino acid sequence that is identical in at least 50%, at least 60% and specifically 65%, 70%, 75%, 80%, 85%, 90%, 91%, 92%, 93%, 94%, 95%, 96%, 97%, 98%, 99%, or higher to SEQ ID NO: 2 and 18 specifically, the entire sequence as denoted by SEQ ID NO: 2 and 18, or any one of the CDRs of SEQ ID NO: 6, 10, 14, 22, 26 and 30. In some embodiments, derivatives refer to antibodies, which differ from the antibodies specifically defined in the present invention by insertions, deletions or substitutions of amino acid residues. It should be appreciated that by the terms “insertion/s”, “deletion/s” or “substitution/s”, as used herein it is meant any addition, deletion or replacement, respectively, of amino acid residues to the polypeptides disclosed by the invention, of between 1 to 50 amino acid residues, between 20 to 1 amino acid residues, and specifically, between 1 to 10 amino acid residues. More particularly, insertion/s, deletion/s or substitution/s may be of any one of 1, 2, 3, 4, 5, 6, 7, 8, 9, or 10 amino acids. It should be noted that the insertion/s, deletion/s or substitution/s encompassed by the invention may occur in any position of the modified peptide, as well as in any of the N or C′ termini thereof. With respect to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologues, and alleles of the invention. For example, substitutions may be made wherein an aliphatic amino acid (G, A, I, L, or V) is substituted with another member of the group, or substitution such as the substitution of one polar residue for another, such as arginine for lysine, glutamic for aspartic acid, or glutamine for asparagine. Each of the following eight groups contains other exemplary amino acids that are conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) Cysteine (C), Methionine (M). More specifically, amino acid “substitutions” are the result of replacing one amino acid with another amino acid having similar structural and/or chemical properties, i.e., conservative amino acid replacements. Amino acid substitutions may be made on the basis of similarity in polarity, charge, solubility, hydrophobicity, hydrophilicity, and/or the amphipathic nature of the residues involved. For example, nonpolar “hydrophobic” amino acids are selected from the group consisting of Valine (V), Isoleucine (I), Leucine (L), Methionine (M), Phenylalanine (F), Tryptophan (W), Cysteine (C), Alanine (A), Tyrosine (Y), Histidine (H), Threonine (T), Serine (S), Proline (P), Glycine (G), Arginine (R) and Lysine (K); “polar” amino acids are selected from the group consisting of Arginine (R), Lysine (K), Aspartic acid (D), Glutamic acid (E), Asparagine (N), Glutamine (Q); “positively charged” amino acids are selected form the group consisting of Arginine (R), Lysine (K) and Histidine (H) and wherein “acidic” amino acids are selected from the group consisting of Aspartic acid (D), Asparagine (N), Glutamic acid (E) and Glutamine (Q). Variants of the antibodies of the invention may have at least 80% sequence similarity or identity, often at least 85% sequence similarity or identity, 90% sequence similarity or identity, or at least 95%, 96%, 97%, 98%, or 99% sequence similarity or identity at the amino acid level, with the protein of interest, such as the antibodies of the invention, specifically, antibodies that comprise the CDRs of SEQ ID NO: 6, 10, 14, 22, 26, 30, or the heavy and light chains as denoted by SEQ ID NO; 2 and 18, respectively. In some embodiments, the invention relates to a biosimilar derived from the mAb-B7 antibody described above. The term “biosimilar” means a biological product that is highly similar to a U.S. licensed reference biological product notwithstanding minor differences in clinically inactive components, and for which there are no clinically meaningful differences between the biological product and the reference product in terms of the safety, purity, and potency of the product. Furthermore, a similar biological or “biosimilar” medicine is a biological medicine that is similar to another biological medicine that has already been authorized for use by the European Medicines Agency. The term “biosimilar” is also used synonymously by other national and regional regulatory agencies. Biological products or biological medicines are medicines that are made by or derived from a biological source, such as a bacterium or yeast. In Europe, a similar biological or “biosimilar” medicine is a biological medicine that is similar to another biological medicine that has already been authorized for use by the European Medicines Agency (EMA). The relevant legal basis for similar biological applications in Europe is Article 6 of Regulation (EC) No 726/2004 and Article 10(4) of Directive 2001/83/EC, as amended and therefore in Europe, the biosimilar may be authorized, approved for authorization or subject of an application for authorization under Article 6 of Regulation (EC) No 726/2004 and Article 10(4) of Directive 2001/83/EC. The already authorized original biological medicinal product may be referred to as a “reference medicinal product” in Europe. Some of the requirements for a product to be considered a biosimilar are outlined in the CHMP Guideline on Similar Biological Medicinal Products. In addition, product specific guidelines, including guidelines relating to monoclonal antibody biosimilars, are provided on a product-by-product basis by the EMA. A biosimilar as described herein may be similar to the reference medicinal product (anti-EspB antibody) by way of quality characteristics, biological activity, mechanism of action, safety profiles and/or efficacy, or any combinations thereof. In addition, the biosimilar may be used or be intended for use to treat the same conditions as the reference medicinal product. Thus, a biosimilar as described herein may be deemed to have similar or highly similar quality characteristics to a reference medicinal product. Alternatively, or in addition, a biosimilar as described herein may be deemed to have similar or highly similar biological activity to a reference medicinal product. Alternatively, or in addition, a biosimilar as described herein may be deemed to have a similar or highly similar safety profile to a reference medicinal product. Alternatively, or in addition, a biosimilar as described herein may be deemed to have similar or highly similar efficacy to a reference medicinal product. As described herein, a biosimilar in Europe is compared to a reference medicinal product which has been authorized by the EMA. However, in some instances, the biosimilar may be compared to a biological medicinal product which has been authorized outside the European Economic Area (a non-EEA authorized “comparator”) in certain studies. Such studies include for example certain clinical and in vivo non-clinical studies. As used herein, the term “biosimilar” also relates to a biological medicinal product which has been or may be compared to a non-EEA authorized comparator. Certain biosimilars are proteins such as antibodies, antibody fragments (for example, antigen binding portions) and fusion proteins. A protein biosimilar may have an amino acid sequence that has minor modifications in the amino acid structure (including for example deletions, additions, and/or substitutions of amino acids) which do not significantly affect the function of the polypeptide. The biosimilar may comprise an amino acid sequence having a sequence identity of 97 percent or greater to the amino acid sequence of its reference medicinal product, e.g., 97 percent, 98 percent, 99 percent or 100 percent. The biosimilar may comprise one or more post-translational modifications, for example, although not limited to, glycosylation, oxidation, deamidation, and/or truncation which is/are different to the post-translational modifications of the reference medicinal product, provided that the differences do not result in a change in safety and/or efficacy of the medicinal product. The biosimilar may have an identical or different glycosylation pattern to the reference medicinal product. Particularly, although not exclusively, the biosimilar may have a different glycosylation pattern if the differences address or are intended to address safety concerns associated with the reference medicinal product. Additionally, the biosimilar may deviate from the reference medicinal product in for example its strength, pharmaceutical form, formulation, excipients and/or presentation, providing safety and efficacy of the medicinal product is not compromised. The biosimilar may comprise differences in for example pharmacokinetic (PK) and/or pharmacodynamic (PD) profiles as compared to the reference medicinal product but is still deemed sufficiently similar to the reference medicinal product as to be authorized or considered suitable for authorization. In certain circumstances, the biosimilar exhibits different binding characteristics as compared to the reference medicinal product, wherein the different binding characteristics are considered by a Regulatory Authority such as the EMA not to be a barrier for authorization as a similar biological product. The term “biosimilar” is also used synonymously by other national and regional regulatory agencies. Still further, in some embodiments, the antibody of the invention may specifically recognize and bind at least one component of the T3SS of at least one bacterium. The “Type III Secretion System or T355” is a complex structure composed of several subunits, which in turn are made up of approximately 20 bacterial proteins. The proteins that make up the T3SS apparatus are termed structural proteins. Additional proteins called “translocators” serve the function of translocating another set of proteins into the host cell cytoplasm. The translocated proteins are termed “effectors,” since they are the virulence factors that affect the changes in the host cells, allowing the invading pathogen to colonize, multiply, and in some cases chronically persist in the host. Briefly, the T3SS apparatus consists of two rings that provide a continuous path across the inner and outer membranes, including the peptidoglycan layer. The inner membrane ring is the larger of the two coaxial rings, and protein components that make up the inner ring have been identified for a number of bacteria. The outer membrane ring is composed of the secretin protein family, which is also known to be involved in type 2 secretion and in the assembly of type IV bacterial pili. A needle-like structure associates with the outer membrane ring and projects from the bacterial surface. It varies in length among the different pathogens and, in the case of pathogenic Escherichia coli, is extended by the addition of filaments that are thought to facilitate attachment to the host cells through the thick glycocalyx layer. Effectors are thought to be transported through the hollow tube-like needle into the host cell through the pores formed in the host cell membrane by the translocator proteins. Translocators are usually conserved among the different pathogens possessing a T3SS and show functional complementarity for secretion and translocation, whereas the effectors are most often distinct, having unique functions suited to a particular pathogen's virulence strategy. However, effector homologues also exist among different T3SS-possessing bacteria. It should be noted that the term “bacterium” or “bacteria” as used herein refers to any of the prokaryotic microorganisms that exist as a single cell or in a cluster or aggregate of single cells. In more specific embodiments, the term “bacteria” specifically refers to Gram negative bacteria, or Gram positive bacteria. In yet some further specific embodiments the term bacteria specifically refers to Gram negative bacteria. While the Gram-positive bacteria are recognized as retaining the crystal violet stain used in the Gram staining method of bacterial differentiation and appear to be purple-colored under a microscope, the Gram-negative bacteria do not retain the crystal violet, making positive identification possible. In other words, the term bacteria apply herein to bacteria with a thin peptidoglycan layer of their cell wall that is sandwiched between an inner cytoplasmic cell membrane and a bacterial outer membrane (Gram-negative). In yet some other embodiments, the bacterium relevant to the antibody of the invention may be at least one Multiple Drug Resistant (MDR) bacterium. “MDR”— “Multiple Drug Resistance” is used in the present invention in respect to Multi Drug Resistant Bacterium. In accordance, it describes an “Anti-Microbial Resistance” (AMR) which is defined as the ability of a microorganism (bacteria, virus, fungi, parasite) to resist the effects of a ding. Microbes resistant to multiple antimicrobials are called multidrug resistant (MDR). AMR occurs when bacteria, viruses, fungi and parasites change over time and no longer respond to medicines, therefore render the medications used to cure the infections they cause ineffective. As used herein, the term “resistance” is not meant to imply that the bacterial cell population is 100% resistant to a specific antibiotic compound, but includes bacteria that are tolerant of the antibiotics or any derivative thereof. More specifically, the term “bacterial resistance gene/s” refers to gene/s conferring about 100%, 90%, 80%, 70%, 60%, 50%, 40%, 30%, 20%, or 10% protection from an antibiotic compound, thereby reversing susceptibility and sensitivity thereof to said antibiotic compound. Bacteria of particular interest may be any bacteria involved in nosocomial infections or any mixture of such bacteria. The term “Nosocomial Infections” refers to Hospital-acquired infections, namely, an infection whose development is favored by a hospital environment, such as surfaces and/or medical personnel, and is acquired by a patient during hospitalization. Nosocomial infections are infections that are potentially caused by organisms resistant to antibiotics. Nosocomial infections have an impact on morbidity and mortality, and pose a significant economic burden. In view of the rising levels of antibiotic resistance and the increasing severity of illness of hospital in-patients, this problem needs an urgent solution. Common nosocomial organisms include Clostridium difficile, methicillin-resistant Staphylococcus aureus, coagulase-negative Staphylococci, vancomycin-resistant Enterococci, resistant Enterobacteriaceae, Pseudomonas aeruginosa, Acinetobacter and Stenotrophomonas maltophilia. The nosocomial-infection pathogens may be Gram-negative rod-shaped (bacilli) organisms (Klebsiella pneumonia, Klebsiella oxytoca, Escherichia coli, Proteus aeruginosa, Serratia spp., Enterobacter aerogenes, Enterobacter cloacae), aerobic Gram-negative coccobacilli (Acinetobacter baumanii, Stenotrophomonas maltophilia) and Gram-negative aerobic bacillus (Stenotrophomonas maltophilia, previously known as Pseudomonas maltophilia). Among many others Pseudomonas aeruginosa is an extremely important nosocomial Gram-negative aerobic rod pathogen. “ESKAPE” pathogens may also be of particular interest. As indicated herein, these pathogens include but are not limited to Enterococcus faecium, Staphylococcus aureus, Klebsiella pneumoniae, Acinetobacter baumanii, Pseudomonas aeruginosa, and Enterobacter. In further embodiments, the bacteria as referred to herein by the invention may include Yersinia enterocolitica, Yersinia pestis, Yersinia pseudotuberculosis, Salmonella bongori, Salmonella typhi that is a sub specie of Salmonella enterica, Pseudomonas aeruginosa, P. aeruginosa, P. oryzihabitans, P. plecoglossicida, P. alcaligenes, P. anguilliseptica, P. argentinensis, P. borbori, P. citronellolis, P. flavescens, P. mendocina, P. nitroreducens, P. oleovorans, P. pseudoalcaligenes, P. resinovorans, P. straminea, Vibrio cholerae, V. parahaemolyticus, and V. vulnificus, Shigella sonnei, S. bovelii, S. dysenteriae, S. fiexneri, Bordetella Pertussis, B. parapertussis, B. bronchiseptica, B. avium, B. hinzii, B. hoimesii, B. trematurn, B. ansorpii and B. petrii, Plasmodium falciparum, Chlamydia trachomatis, C. muridarum, C. pneumoniae, C. pecorum, C. suis, C. abortus, C. fells, C. curiae, and C. psittaci, Bacillus anthracis, B. cereus, B. thuringiensis, B. weihenstephanensis, B. mycoides, and B. pseudomycoides, Helicobacter pylori and Listeria monocytogens, L. fleischmannii, L. grayi, L. innocua, L. ivanovii, L marthii, L. rocourtiae, L. seeligeri, L. weihenstephanensis, and L. welshimeri. L denitrificans. In some other specific embodiments, the bacterium referred herein may be a gram negative. In other specific embodiments, the target cells of interest may be any E. coli strain, specifically, any one of enteroaggregative (EAEC), enterohemorrhagic (EHEC), enteroinvasive (EIEC), enteropathogenic (EPEC), enterotoxigenic (ETEC) and diffuse adherent (DAEC) E. coli. In some further embodiments, the MDR bacterium relevant to the antibody of the invention may be least one of EPEC and EHEC. EPEC and EHEC (e.g., O157:H7) are the main bacterial agents associated with diarrhea among children under 5 years old, and both pathogens are able to induce the A/E lesion. In some embodiments, the MDR bacterium may be EPEC. In some other embodiments, the MDR bacterium may be C. rodentium. In some further embodiments, the antibody of the invention may bind the Escherichia Coli secreted protein B (EspB) expressed by the bacterium. Among the virulence factors comprising the T3SS of these bacteria are the secreted proteins (Esps). The Esp responsible for the syringe-like structure of T3SS is secreted protein A (EspA), which is the needle-shaped protein of approximately 25 kDa, while secreted proteins B [Escherichia coli-secreted protein B (EspB)] and D [Escherichia coli-secreted protein D (EspD)] are responsible for the pore structure assembled in the eukaryotic membrane. EspB is approximately 34 kDa in size and forms a pore (translocon) in the host cell membrane together with EspD. Also, EspB participates in phagocytosis evasion and binding to eukaryotic cell myosin, inhibition of actin interaction, and damage to the microvilli. There are three variants of EspB, i.e., α, β, and γ, where the α variant is subdivided into 1, 2, and 3. Allele frequency studies have shown a EspB to be the most prevalent, followed by (3 EspB. In some embodiment, the EspB protein comprises the amino acid sequence as denoted by SEQ ID NO: 40 (Accession number: WP_001091991.1), or any variants, homologs or derivatives thereof. In some specific embodiments, the Esp protein is encoded by a nucleic sequence as denoted by SEQ ID NO: 41 (Accession number: AAB69980.1), or any homologs or derivatives thereof. In some specific embodiments, homolog as used herein may include the EHEC EspB as dented by SEQ ID NO: 45, and/or the C. rodentium as dented by SEQ ID NO: 46. In some further embodiments, the EspD protein comprises the amino acid sequence as denoted by SEQ ID NO: 42 (Accession number: WP_000935767.1), or any homologs or derivatives thereof. In some specific embodiments, the Esp protein is encoded by a nucleic sequence as denoted by SEQ ID NO: 43 (Accession number: CAI43861.1). In some embodiments, the isolated antibody of the invention, specifically recognizes and binds an epitope comprising residues 185 to 250, specifically residues 190 to 215, more specifically, residues 193 to 210, of the EspB protein, specifically, the EspB protein that comprises the amino acid sequence as denoted by SEQ ID NO: 40. In yet some further embodiments, the epitope recognized by the antibody of the invention may comprise the amino acid sequence of TSAQKASQVAEEAADAAQ, or at least part thereof. In yet some further embodiments, the epitope recognized by the antibody of the invention may comprise the amino acid sequence as denoted by SEQ ID NO: 39. In some embodiments, the mAb-B7 antibody recognized the epitope as denoted by SEQ ID NO: 49, that is a fragment of SEQ ID NO: 39. Still further, the mAb-B7 antibody further recognizes the residues of peptide #9-12, as denoted by SEQ ID NO: 48. Thus, in some embodiments, the present disclosure further encompasses any antibody that recognizes and binds the epitope of at least one of SEQ ID NO; 39, 48 and 49, or any combinations thereof. In yet some further embodiments, the antibody of the invention may interfere with or inhibit at least one of the assembly and the secretion of at least one component of the T3SS. In another embodiment, the antibody of the invention may interfere with the assembly and/or the secretion of the EspB protein and the EspD protein of the bacterium. In yet some further alternative embodiments, the antibody of the invention does not interfere with or inhibit the assembly and the secretion the EspB protein and the EspD protein. In yet some further embodiments, the antibody of the invention modulates the immune system of a subject infected with at least one T3SS expressing bacteria. In some embodiments, the antibody of the present disclosure promotes certain activities of the immune system against bacteria, such as opsonization and phagocytic clearance. These activities may prevent the spread of the bacterial infection within the host body and induce a humoral response with serological memory that will shorten the infection duration, promote recovery and provide cellular and serological memory. Thus, in some embodiments the antibodies of the invention may induce at least one of a humoral response and serological memory in said subject. In some embodiments, the antibodies of the invention may induce opsonization. More specifically, antibody opsonization is a process by which a pathogen is marked for destruction by antibody-dependent cellular cytotoxicity (ADCC), antibody-dependent cellular phagocytosis (ADCP), or complement-dependent cytotoxicity (CDC). More specifically, pathogens that may be any antigens, molecules or microorganisms, are coated with host-produced proteins and lipids (immunoglobulins, complement factors), thereby facilitating the binding and recognition of the opsonized particle or bacteria, by specific receptor molecules present on phagocytes. Thus, in yet some further embodiments, the antibody of the invention may enhance phagocytic clearance of the T3SS expressing bacteria in the infected subject. The antibody of the present disclosure therefore modulates the immune system of the subject. After a primary immune response against an antigen has occurred, re-exposure to the same antigen induces a secondary, much more rapid and robust recall immune response that completely protects the host from, or at least reduces the extent of the reinfection. The development of immunological memory is a hallmark of the adaptive immune system and is mediated by another main aspect of the adaptive immunity—the humoral immune response. In some embodiments, the antibody induces at least one of a humoral response and immunological and/or serological memory in a subject. More specifically, the humoral immune response encompasses serum antibodies and the cells involved in their production (i.e., memory β-cells and long-lived plasma cells, LLPCs), that were created in the body following the primary immune response, and remain long after the primary immune response has disappeared and the effector cells of the immune response are eliminated. The immune component of the immunological memory, comprise an important defensive mechanism in subsequent infections. it confers protection in the extracellular space, mediated by secreted antibodies, allowing the direct: inactivation, or killing of micro-organisms via, for example, neutralization or opsonization. The other component of the immunological memory being the cellular component involves T-calls (CD4 and CD8) that produce cytokines and kill already infected cells. More specifically, the immune system memorizes the characteristics of pathogens to provide effective immune protection. Memory B cells and long-lived plasma cells (PCs) account for the long-term humoral immunity elicited by infections. Once generated, memory B cells enter a resting state and persist over long periods of time in the lymphoid organs in the apparent absence of immunizing antigen. T cell-dependent (TD) B cell memory is generated along two fundamentally distinct differentiation pathways. One of these is the classical generation pathway through antibody affinity maturation in the germinal center (GC) reaction with the help of T follicular helper (Tfh) cells. In the other pathway, memory B cells develop in response to a TD antigen before the onset and independently of the GC reaction with the help of T cells. The maintenance of these cells over time may depend on interactions with cells in their environment, perhaps in specialized “niches” akin to those postulated for the maintenance of PCs. Memory B cells provide the quick anamnestic antibody response that follows after antigen re-exposure. This activity is critical for eliminating pathogens. Still further, it should be understood, that in some embodiments thereof, the invention further encompasses any antibody that competes with any of the antibodies of the invention, specifically, any antibody that competes with an antibody comprising at least one of the CDRs as denoted by SEQ ID NO: 6, 10, 14, 22, 26 and 30, or any homologs or derivatives thereof. In yet some further embodiments, the invention further encompasses any antibody that competes with an antibody comprising the variable heavy chain as denoted by SEQ ID NO: 2, or any homologs or derivatives thereof, and/or the variable light chain that comprises the amino acid sequence as denoted by SEQ ID NO: 18, or any homologs or derivatives thereof. In yet some further embodiments, the term “competes” as used herein refers to any competition that results in reduction, attenuation, decrease or inhibition of binding of at least one of, the binding of the antibody of the invention to its epitope, and interference with the induction of a humoral response and serological memory in a subject. Thus, any antibody that competes with the antibody of the invention in at least one of these effects, is also encompassed by the present invention. Still further, any antibody that binds the epitope of SEQ ID NO: 39, 49 and 48, or any combinations thereof, is encompassed by the preset disclosure. In some other embodiments, the isolated monoclonal antibody according to the invention may be for use in a method of treating, preventing, ameliorating, reducing or delaying the onset of an infection by at least one bacterium expressing a T3SS in a subject. A further aspect of the invention therefore provides a pharmaceutical composition comprising as an active ingredient an antibody or any antigen-binding fragment thereof, or any matrix, nano- or micro-particles thereof, wherein the antibody comprises at least one heavy chain CDRH1 comprising the amino acid sequence GFTFSHYA, as denoted by SEQ ID NO: 6, or any homologs or derivatives thereof, CDRH2 comprising the amino acid sequence INSNGDST, as denoted by SEQ ID NO: 10, or any homologs or derivatives thereof, CDRH3 comprising the amino acid sequence ARDRRAGYFDYW, as denoted by SEQ ID NO: 14, or any homologs or derivatives thereof, and at least one light chain CDRL1 comprising the amino acid sequence RDNIGKNY as denoted by SEQ ID NO: 22, or any homologs or derivatives thereof, CDRL2 comprising the amino acid sequence RNN as denoted by SEQ ID NO: 26, or any homologs or derivatives thereof, and CDRL3 comprising the amino acid sequence SAWDTSLNA as denoted by SEQ ID NO: 30, or any homologs or derivatives thereof, or any derivative, variant and biosimilar thereof, said composition optionally further comprises at least one of a pharmaceutically acceptable carrier, excipient and diluent. In some embodiments, the pharmaceutical composition of the invention may comprise any of the antibodies of the invention, specifically, any of the antibodies as detailed above.

As noted above, the pharmaceutical composition of the invention may optionally further comprise at least one pharmaceutically acceptable carrier/s, excipient/s, auxiliaries, and/or diluent/s. “Pharmaceutically or therapeutically acceptable carrier” refers to a carrier medium which does not interfere with the effectiveness of the biological activity of the active ingredients. As mentioned herein, the compositions provided by the invention optionally further comprise at least one pharmaceutically acceptable excipient or carrier. As used herein “pharmaceutically acceptable carrier” includes any and all solvents, dispersion media, coatings, antibacterial and antifungal agents and the like. The use of such media and agents for pharmaceutical active substances is well known in the art. Except as any conventional media or agent is incompatible with the active ingredient, its use in the therapeutic composition is contemplated. As used herein “pharmaceutically acceptable carrier/diluents/excipient” includes any and all solvents, dispersion media, coatings and the like. The use of such media and agents for pharmaceutical active substances is well known in the art. Except as any conventional media or agent is incompatible with the active ingredient, its use in the therapeutic composition is contemplated. Pharmaceutical compositions used to treat subjects in need thereof according to the invention generally comprise a buffering agent, an agent who adjusts the osmolarity thereof, and optionally, one or more pharmaceutically acceptable carriers, excipients and/or additives as known in the art. Supplementary active ingredients can also be incorporated into the compositions. The carrier can be solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. Excipients of the present invention can include, without limitation, lipidoids, liposomes, lipid nanoparticles, polymers, lipoplexes, core-shell nanoparticles. In various embodiments, the final solution of any of the compositions of the invention may be adjusted with a pharmacologically acceptable acid, base or buffer. In some embodiments, the antibodies of the invention or any compositions, combinations or kits thereof may be adapted for systemic administration. The antibody, pharmaceutical composition and kits of the invention can be administered and dosed by the methods of the invention, in accordance with good medical practice. More specifically, the antibody, compositions and combinations used in the methods and kits of the invention, described herein after, may be adapted for administration by systemic, parenteral, intraperitoneal, transdermal, oral (including buccal or sublingual), rectal, topical (including buccal or sublingual), vaginal, intranasal and any other appropriate routes. Such formulations may be prepared by any method known in the art of pharmacy, for example by bringing into association the active ingredient with the carrier(s) or excipient(s). In some embodiments, the antibodies of the invention or any compositions, combinations or kits thereof may be suitable for systemic administration. The antibody, pharmaceutical composition and kits of the invention can be administered and dosed by the methods of the invention, in accordance with good medical practice. More specifically, the antibody, compositions and combinations used in the methods and kits of the invention, described herein after, may be adapted for administration by systemic, parenteral, intraperitoneal, transdermal, oral (including buccal or sublingual), rectal, topical (including buccal or sublingual), vaginal, intranasal and any other appropriate routes. Such formulations may be prepared by any method known in the art of pharmacy, for example by bringing into association the active ingredient with the carrier(s) or excipient(s). In yet another aspect, the invention provides an isolated nucleic acid molecule comprising a nucleotide sequence encoding the antibody of the invention or any antigen-binding fragment thereof, and any matrix, nano- or micro-particles thereof. The term “nucleic acid”, “nucleic acid sequence”, or “polynucleotide” and “nucleic acid molecule” refers to polymers of nucleotides, and includes but is not limited to deoxyribonucleic acid (DNA), ribonucleic acid (RNA), DNA/RNA hybrids including polynucleotide chains of regularly and/or irregularly alternating deoxyribosyl moieties and ribosyl moieties (i.e., wherein alternate nucleotide units have an —OH, then and —H, then an —OH, then an —H, and so on at the 2′ position of a sugar moiety), and modifications of these kinds of polynucleotides, wherein the attachment of various entities or moieties to the nucleotide units at any position are included. The terms should also be understood to include, as equivalents, analogs of either RNA or DNA made from nucleotide analogs, and, as applicable to the embodiment being described, single-stranded (such as sense or antisense) and double-stranded polynucleotides. Preparation of nucleic acids is well known in the art. Still further, in some embodiments, the invention provides an expression vector or cassette comprising any of the isolated nucleic acid molecules of the invention, specifically, any of the nucleic acid molecules encoding any of the antibodies of the invention. It should be further appreciated that the invention also encompasses host cell/s transformed or transfected with any of the expression vectors of the invention that comprise nucleic acid sequences encoding any of the antibodies of the invention. In yet another aspect, the invention relates to a method of treating, preventing, ameliorating, reducing or delaying the onset of an infection by at least one bacterium expressing at least one T3SS in a subject in need thereof. More specifically, the method of the invention comprises the step of administering to the subject a therapeutically effective amount of at least one monoclonal antibody or any antigen-binding fragment thereof, any matrix, nano- or micro-particles thereof, or of any composition comprising the antibody of the invention. More specifically, the antibody of the invention comprises at least one heavy chain CDRH1 comprising the amino acid sequence GFTFSHYA, as denoted by SEQ ID NO: 6, or any homologs or derivatives thereof, CDRH2 comprising the amino acid sequence INSNGDST, as denoted by SEQ ID NO: 10, or any homologs or derivatives thereof, CDRH3 comprising the amino acid sequence ARDRRAGYFDYW, as denoted by SEQ ID NO: 14, or any homologs or derivatives thereof, and at least one light chain CDRL1 comprising the amino acid sequence RDNIGKNY as denoted by SEQ ID NO: 22, or any homologs or derivatives thereof, CDRL2 comprising the amino acid sequence RNN as denoted by SEQ ID NO: 26, or any homologs or derivatives thereof, and CDRL3 comprising the amino acid sequence SAWDTSLNA as denoted by SEQ ID NO: 30, or any homologs or derivatives thereof, or any derivative, variant and biosimilar of the antibodies of the invention. In some embodiments, the antibody suitable for the method of the invention may comprise a heavy chain variable region and a light chain variable region, wherein the heavy chain variable region is encoded by a nucleic acid sequence which is at least 70% identical to the nucleic acid sequence denoted by SEQ ID NO: 1 and wherein the light chain variable region is encoded by a nucleic acid sequence which is at least 70% identical to SEQ ID NO: 17, or any homologs or derivatives thereof. In some embodiments, the antibody suitable for the method of the invention may comprise a heavy chain variable region comprising the amino acid sequence denoted by SEQ ID NO: 2, or any homologs, derivatives or variants thereof and a light chain variable region comprising the amino acid sequence denoted by SEQ ID NO: 18, or any homologs, derivatives or variants thereof. In some embodiments, the antibody suitable for the method of the invention may comprise a Heavy chain Framework Region 1 (FR1) comprising the amino acid sequence denoted by SEQ ID NO: 4 or any homologs or derivatives thereof, a heavy chain FR2 comprising the amino acid sequence denoted by SEQ ID NO: 8, or any homologs or derivatives thereof, and a heavy chain FR3 comprising the amino acid sequence denoted by SEQ ID NO: 12, or any homologs or derivatives thereof, and a Light chain Framework Region 1 (FR1) comprising the amino acid sequence denoted by SEQ ID NO: 20, or any homologs or derivatives thereof, a Light chain FR2 comprising the amino acid sequence denoted by SEQ ID NO: 24 or any homologs or derivatives thereof, and a Light chain FR3 comprising the amino acid sequence denoted by SEQ ID NO: 28, or any homologs or derivatives thereof. It should be noted that in some embodiments, the methods of the invention may use any antibody that recognizes and binds the epitope recognized by the antibody of the invention. In yet some further embodiments, any antibody that competes with the antibody may be used by the methods of the invention. More specifically, any antibody that competes with an antibody comprising at least one of the CDRs as denoted by SEQ ID NO: 6, 10, 14, 22, 26 and 30, or any homologs or derivatives thereof. As noted above, the invention further encompasses methods using any antibody that competes with any of the antibodies of the invention, specifically, any antibody that competes with an antibody comprising at least one of the CDRs as denoted by SEQ ID NO: 6, 10, 14, 22, 26 and 30, or any homologs or derivatives thereof. In yet some further embodiments, the invention further encompasses methods using any antibody that competes with an antibody comprising the variable heavy chain as denoted by SEQ ID NO: 2, or any homologs or derivatives thereof, and/or the variable light chain that comprises the amino acid sequence as denoted by SEQ ID NO: 18, or any homologs or derivatives thereof. In some specific embodiments, the antibody suitable for the method of the invention may specifically recognize and bind at least one component of the T3SS of at least one bacterium. In more specific embodiments, the antibody used by the methods of the invention recognizes and binds at least one component of the bacterial T3SS. More specifically, the bacteria referred to a gram negative bacteria. In some further embodiments, the antibody used by the methods of the invention recognizes and binds at least one component of the T3SS of at least one MDR bacterium. In some further embodiments, the MDR bacterium may be at least one of EPEC and EHEC. Specifically concerning the EPEC and EHEC bacteria, the hallmark of EPEC and EHEC-induced intestinal pathology is the attaching and effacing (A/E) lesion, whose formation depends on a T3SS encoded within the loci of enterocyte effacement (LEE) and the interplay of many T3SS effectors. Following intimate attachment of the bacteria to the intestinal epithelium, the brush border microvilli are disrupted (effacement), and the bacteria promote formation of actin pedestals that elevate the pathogen above the intestinal epithelium. To attach to the enterocytes, EPEC and EHEC utilize their T3SSs to inject the Translocated Intimin Receptor (Tir) into the host cell, where it inserts into the host cell membrane and binds to the bacterial outer membrane protein intimin. Binding of intimin to Tir induces Tir clustering, initiating a cascade of signaling events that leads to actin polymerization and pedestal formation. This ultimately results in the formation of the A/E lesion. EPEC Tir is tyrosine phosphorylated to recruit the Arp2/3 complex and drive actin polymerization, whereas EHEC Tir is not phosphorylated but, rather, relies on an additional T3SS effector, TccP/EspFU, for Arp2/3 recruitment. Successful pedestal formation requires downregulation of filopodia, which form in response to EPEC/EHEC infection, as well as disruption of the host microtubule network. The T3SS effectors Map (mitochondrion-associated protein), Tir, EspH (153), EspG, and EspG2 mediate these processes. This multifaceted approach allows A/E pathogens to coordinate the formation of A/E lesions and actin pedestals, providing them with a unique niche in the intestine of the infected host. In some more specific embodiments, the T3SS recognized by the antibody used by the methods of the invention may be an MDR bacterium, in some specific embodiments, such bacteria may be EPEC. In yet another embodiment, the bacteria may induce attaching and effacing (A/E) lesion in the subject. In some further embodiments, the bacteria referred herein may be C. rodentium. In some embodiments, the antibody used by the methods of the invention modulates the immune system of the subject. In yet some further embodiments, the antibody used by the methods of the invention induces at least one of a humoral response and immunological memory in the subject. As noted above, the methods of the invention are applicable for treatment and prevention of disorders associated with T3SS-expressing pathogens. The clinical spectrum of disease caused by T3SS-containing pathogens is remarkably broad. Infection with EPEC, EHEC, Shigella, Salmonella, and Yersinia species results in intestinal disease. Yersinia pestis is the causative agent of plague. Salmonella serovar Typhi causes enteric fever. Bordetella causes whooping cough, while the opportunistic pathogen Pseudomonas aeruginosa can cause a variety of problems, including pneumonia, urinary tract infection, wound infection, septicemia, and endocarditis. Chlamydia trachomatis is a common sexually transmitted organism, and Chlamydia pneumoniae causes pneumonia and has been implicated in atherosclerotic disease of blood vessels. Burkholderia pseudomallei causes community-acquired bacteremia and pneumonia. Whether by a direct toxic mechanism or through induction of self-damaging host responses, the virulence of all of these bacteria utilizes T3SSs. Clearly, T3SSs are not restricted to a specific pathogen, tissue, host environment, clinical disease spectrum, or patient population. In some further embodiments, the methods of the invention may be applicable for treating any infection associated with at least one of transient enteritis or colitis, cholecystitis, bacteremia, cholangitis, urinary tract infection (UTI), traveler's diarrhea, neonatal meningitis and pneumonia, or any conditions, symptoms or effects associated therewith. Thus, in some specific embodiments, the methods of the invention may be applicable for transient enteritis. The term “transient enteritis or colitis” relates to an inflammation of the small intestine. It is most commonly caused by food or drink contaminated with pathogenic microbes. Duodenitis, jejunitis and ileitis are subtypes of enteritis which are only localized to a specific part of the small intestine. Inflammation of both the stomach and small intestine is referred to as gastroenteritis. Signs and symptoms of enteritis are highly variable and vary based on the specific cause and other factors such as individual variance and stage of disease. Symptoms may include abdominal pain, cramping, diarrhoea, dehydration, fever, nausea, vomiting and weight loss. In yet some further embodiments, the methods of the invention may be applicable for Cholecystitis. As used herein, Cholecystitis is inflammation of the gallbladder. Symptoms include right upper abdominal pain, nausea, vomiting, and occasionally fever. Often gallbladder attacks (biliary colic) precede acute cholecystitis. Complications of acute cholecystitis include gallstone pancreatitis, common bile duct stones, or inflammation of the common bile duct. In some further embodiments, the methods of the invention may be applicable for Bacteremia. Bacteremia (also bacteraemia) refers to the presence of bacteria in the blood. Bacteria can enter the bloodstream as a severe complication of infections (like pneumonia or meningitis), during surgery (especially when involving mucous membranes such as the gastrointestinal tract), or due to catheters and other foreign bodies entering the arteries or veins (including during intravenous drug abuse). Transient bacteremia can result after dental procedures or brushing of teeth. Bacteremia can have several important health consequences. The immune response to the bacteria can cause sepsis and septic shock, which has a high mortality rate. Bacteria can also spread via the blood to other parts of the body (which is called hematogenous spread), causing infections away from the original site of infection, such as endocarditis or osteomyelitis. Still further, in some embodiments, the methods of the invention may be applicable for cholangitis. Ascending cholangitis, also known as acute cholangitis or cholangitis, is inflammation of the bile duct, usually caused by bacteria ascending from its junction with the duodenum (first part of the small intestine). It tends to occur if the bile duct is already partially obstructed by gallstones. Characteristic symptoms include yellow discoloration of the skin or whites of the eyes, fever, abdominal pain, and in severe cases, low blood pressure and confusion. In yet some further embodiments, the methods of the invention may be applicable for urinary tract infection. A urinary tract infection (UTI) is an infection that affects part of the urinary tract. When it affects the lower urinary tract it is known as a bladder infection (cystitis) and when it affects the upper urinary tract it is known as kidney infection (pyelonephritis). Symptoms from a lower urinary tract include pain with urination, frequent urination, and feeling the need to urinate despite having an empty bladder. Symptoms of a kidney infection include fever and flank pain usually in addition to the symptoms of a lower UTI. In some cases, the urine may appear bloody. In certain embodiments, the methods of the invention may be applicable for Traveler's diarrhea. Traveler's diarrhea (TD) is a stomach and intestinal infection. TD is defined as the passage of unformed stool (one or more by some definitions, three or more by others) while traveling. It may be accompanied by abdominal cramps, nausea, fever, and bloating. Occasionally bloody diarrhea may occur. Most travelers recover within four days with little or no treatment. About 10% of people may have symptoms for a week. Bacteria are responsible for more than half of cases. The bacteria enterotoxigenic Escherichia coli (ETEC) are typically the most common except in Southeast Asia, where Campylobacter is more prominent. In yet some more embodiments, the methods of the invention may be applicable for Neonatal meningitis. Neonatal meningitis is a serious medical condition in infants. Meningitis is an inflammation of the meninges (the protective membranes of the central nervous system (CNS)) and is more common in the neonatal period (infants less than 44 days old) than any other time in life and is an important cause of morbidity and mortality globally. Symptoms seen with neonatal meningitis are often unspecific that may point to several conditions, such as sepsis (whole body inflammation). These can include fever, irritability, and dyspnea. The only method to determine if meningitis is the cause of these symptoms is lumbar puncture (LP; an examination of the cerebrospinal fluid). The most common causes of neonatal meningitis is bacterial infection of the blood, known as bacteremia (specifically Group B Streptococci (GBS; Streptococcus agalactiae), Escherichia coli, and Listeria monocytogenes). Delayed treatment of neonatal meningitis may cause include cerebral palsy, blindness, deafness, and learning deficiencies. In some embodiments, the methods of the invention may be applicable for Pneumonia. Pneumonia is an inflammatory condition of the lung affecting primarily the small air sacs known as alveoli. Typically symptoms include some combination of productive or dry cough, chest pain, fever, and trouble breathing. Bacteria are the most-common cause of community-acquired pneumonia (CAP), with Streptococcus pneumoniae isolated in nearly 50% of cases. Other commonly-isolated bacteria include Haemophilus influenzae in 20%, Chlamydophila pneumoniae in 13%, and Mycoplasma pneumoniae in 3% of cases; Staphylococcus aureus; Moraxella catarrhalis; Legionella pneumophila; and Gram-negative bacilli. A number of drug-resistant versions of the above infections are becoming more common, including drug-resistant Streptococcus pneumoniae (DRSP) and methicillin-resistant Staphylococcus aureus (MRSA). In yet some other embodiments, the method of the invention may further comprise the step of administering to the subject a therapeutically effect amount of at least one anti-bacterial agent. The invention thus further provides combined therapy combining the antibodies of the invention and at least one further antibacterial agent. In some embodiments, the antibacterial agent may be at least one antibiotic agent or any combinations thereof. Thus, in a further aspect, the invention relates to a method of treating, preventing, ameliorating, reducing, or delaying the onset of an infection by at least one bacterium expressing at least one T3SS comprising the step of administering to a subject treated with at least one anti-bacterial agent, a therapeutically effective amount of an isolated antibody or any antigen-binding fragment thereof, or any matrix, nano- or micro-particles thereof, or any composition comprising the antibody. In some specific embodiments, the antibody comprises at least one heavy chain CDRH1 comprising the amino acid sequence GFTFSHYA, as denoted by SEQ ID NO: 6, or any homologs or derivatives thereof, CDRH2 comprising the amino acid sequence INSNGDST, as denoted by SEQ ID NO: 10, or any homologs or derivatives thereof, CDRH3 comprising the amino acid sequence ARDRRAGYFDYW, as denoted by SEQ ID NO: 14, or any homologs or derivatives thereof, and at least one light chain CDRL1 comprising the amino acid sequence RDNIGKNY as denoted by SEQ ID NO: 22, or any homologs or derivatives thereof, CDRL2 comprising the amino acid sequence RNN as denoted by SEQ ID NO: 26, or any homologs or derivatives thereof, and CDRL3 comprising the amino acid sequence SAWDTSLNA as denoted by SEQ ID NO: 30, or any homologs or derivatives thereof, or any derivative, variant and biosimilar thereof. As specified herein above, in connection with previous aspects of the invention, in some embodiments, the methods of the invention may also use any of the antibodies specified above, as well as any antibody that competes with the antibodies of the invention, and/or any antibody that recognizes and binds the epitope of at least one of SEQ ID NO; 39, 48 and 49, or any combinations thereof. In some specific embodiments, the methods of the invention may be applicable for infections caused by MDR bacteria. In some more specific embodiments, the bacteria referred to herein may be a gram negative bacteria. In yet some other embodiments, the bacteria may be at least one of EPEC and EHEC. In other specific embodiments, the methods of the invention are applicable for infectious caused by EPEC. In some embodiments, the infections relevant to the method of the invention may be associated with at least one of transient enteritis or colitis, cholecystitis, bacteremia, cholangitis, UTI, traveler's diarrhea, neonatal meningitis and pneumonia, or any condition, symptoms or effects associated therewith. In some other embodiments, the methods of the invention provide combined therapy using any of the antibodies of the invention as discussed above, and at least one antibacterial agent. In some embodiments, such agent may be at least one antibiotic agent or any combinations thereof. It should be understood that the antibiotic agents relevant to this aspect are described herein after in connection with other aspects of the invention. In some other embodiments, the antibody suitable for the methods of the invention may bind the EspB expressed by the bacterium. In yet some further embodiments, the antibody of the methods of the invention modulates the immune system of the subject. Still further, in some embodiments, the antibody induces at least one of a humoral response and immunological memory in the subject. As indicated above, the invention provides therapeutic methods for treating and inhibiting infectious diseases. In some particular embodiments, the antibody suitable for the method of the invention may interfere with or inhibit at least one of the assembly and the secretion of at least one component of the T3SS. In some yet particular embodiments, the antibody suitable for the method of the invention may interfere with the assembly and/or the secretion of the EspB protein and the EspD protein of the bacterium. In yet some further alternative embodiments, the antibody of the invention does not interfere with or inhibit the assembly and the secretion the EspB protein and the EspD protein. The terms “treat, treating, treatment” as used herein and in the claims mean ameliorating one or more clinical indicia of disease activity by administering a pharmaceutical composition of the invention in a patient having a pathologic disorder. The term “treatment” as used herein refers to the administering of a therapeutic amount of the composition of the present invention which is effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease form occurring or a combination of two or more of the above. The term “prevention” as used herein, includes the prevention or postponement of development of the disease, prevention or postponement of development of symptoms and/or a reduction in the severity of such symptoms that will or are expected to develop, preventing the occurrence or reoccurrence of the acute disease attacks. These further include ameliorating existing symptoms, preventing—additional symptoms and ameliorating or preventing the underlying metabolic causes of symptoms. The term “amelioration” as referred to herein, relates to a decrease in the symptoms, and improvement in a subject's condition brought about by the compositions and methods according to the invention, wherein said improvement may be manifested in the forms of inhibition of pathologic processes associated with the infectious disease caused by a T3SS expressing MDR bacteria described herein, a significant reduction in their magnitude, or an improvement in a diseased subject physiological state. The term “inhibit” and all variations of this term is intended to encompass the restriction or prohibition of the progress and exacerbation of pathologic symptoms or a pathologic process progress, said pathologic process symptoms or process are associated with. The term “eliminate” relates to the substantial eradication or removal of the pathologic symptoms and possibly pathologic etiology, optionally, according to the methods of the invention described below. The terms “delay”, “delaying the onset”, “retard” and all variations thereof are intended to encompass the slowing of the progress and/or exacerbation of a pathologic disorder or an infectious disease and their symptoms slowing their progress, further exacerbation, or development, so as to appear later than in the absence of the treatment according to the invention. More specifically, treatment or prevention include the prevention or postponement of development of the disease, prevention or postponement of development of symptoms and/or a reduction in the severity of such symptoms that will or are expected to develop. These further include ameliorating existing symptoms, preventing additional symptoms and ameliorating or preventing the underlying metabolic causes of symptoms. It should be appreciated that the terms “inhibition”, “moderation”, “reduction” or “attenuation” as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%. With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively. It should be understood that the therapeutic methods of the invention involve any applicable mode of administration. The phrases “systemic administration”, “administered systemically” as used herein mean the administration of a compound, drug or other material other than directly into the central blood system, such that it enters the patient's system and, thus, is subject to metabolism and other like processes. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Systemic administration includes parenteral injection by intravenous bolus injection, by intravenous infusion, by sub-cutaneous, intramuscular, intraperitoneal injections or by suppositories, by patches, or by any other clinically accepted method, including tablets, pills, lozenges, pastilles, capsules, drinkable preparations, ointment, cream, paste, encapsulated gel, patches, boluses, or sprayable aerosol or vapors containing these complexes and combinations thereof, when applied in an acceptable carrier. Alternatively, to any pulmonary delivery as by oral inhalation such as by using liquid nebulizers, aerosol-based metered dose inhalers (WI's), or dry powder dispersion devices. In other embodiments, the antibodies of the invention as well as the pharmaceutical compositions, combinations and kits disclosed herein may be adapted for topical administration. By “topical administration” it is meant that the antibody or any pharmaceutical composition thereof and the carrier may be adapted to any mode of topical administration including: epicutaneous, transdermal, oral, bronchoalveolar lavage, ophtalmic administration, enema, nasal administration, administration to the ear, administration by inhalation. Regardless of the route of administration selected, the compositions of the present invention, which may be used in a suitable hydrated form, and/or the pharmaceutical compositions of the present invention, are formulated into pharmaceutically-acceptable dosage forms by conventional methods known to those of skill in the art. The invention provides methods for treating infectious diseases caused by bacterial infections. As used herein, “disease”, “disorder”, “condition” and the like, as they relate to a subject's health, are used interchangeably and have meanings ascribed to each and all of such terms. It is understood that the interchangeably used terms “associated” and “related”, when referring to pathologies herein, mean diseases, disorders, conditions, or any pathologies which at least one of: share causalities, co-exist at a higher than coincidental frequency, or where at least one disease, disorder, condition or pathology causes a second disease, disorder, condition or pathology. By “patient”, “individual” or “subject” it is meant any organism who may be affected by the above-mentioned conditions, and to whom the prognostic methods herein described are desired, including humans. More specifically, the methods, devices and kits of the invention described herein after, is intended for mammals. By “mammalian subject” is meant any mammal for which the proposed therapy is desired, including human, equine, canine, and feline subjects, most specifically humans. As noted above, the subjects are treated with at least one antibody according to the invention. The term “treatment” refers to the complete range of therapeutically positive effects of administrating to a subject including inhibition, reduction of, alleviation of, and relief from, a condition known to be treated with an antibody of the invention, for example an infectious disease caused by a T3SS expressing MDR bacteria as detailed herein. More specifically, treatment or prevention of relapse or recurrence of the disease includes the prevention or postponement of development of the disease, prevention or postponement of development of symptoms and/or a reduction in the severity of such symptoms that will or are expected to develop. These further include ameliorating existing symptoms, preventing additional symptoms and ameliorating or preventing the underlying metabolic causes of symptoms. It should be appreciated that the terms “inhibition”, “moderation”, “reduction” or “attenuation” as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%. With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively. As noted above, the present invention further encompasses combined therapy using the antibody of the invention with at least one additional therapeutic agent. It should be appreciated that the antibody of the invention may be administered prior to, after and/or simultaneously with administration of the additional therapeutic agent that may be in some embodiments, at least one anti-bacterial agent. The invention thus encompasses combined therapeutic methods, as well as combined kits that allow the administration of the antibody of the invention and the additional agent via separate compositions, administration modes and regimens. To facilitate such combined therapy, the invention provides a kit. Thus, in a further aspect, the invention provides a kit comprising: in a first element (a), at least one antibody or any antigen-binding fragment thereof, or any matrix, nano- or micro-particles thereof. In some embodiments, the antibody comprises at least one heavy chain CDRH1 comprising the amino acid sequence GFTFSHYA, as denoted by SEQ ID NO: 6, or any homologs or derivatives thereof, CDRH2 comprising the amino acid sequence INSNGDST, as denoted by SEQ ID NO: 10, or any homologs or derivatives thereof, CDRH3 comprising the amino acid sequence ARDRRAGYFDYW, as denoted by SEQ ID NO: 14, or any homologs or derivatives thereof, and at least one light chain CDRL1 comprising the amino acid sequence RDNIGKNY as denoted by SEQ ID NO: 22, or any homologs or derivatives thereof, CDRL2 comprising the amino acid sequence RNN as denoted by SEQ ID NO: 26, or any homologs or derivatives thereof, and CDRL3 comprising the amino acid sequence SAWDTSLNA as denoted by SEQ ID NO: 30, or any homologs or derivatives thereof, or any derivative, variant and biosimilar of the antibodies of the invention. In a second element, (b), the kit of the invention further comprises at least one anti-bacterial agent. In some embodiments, the antibody of the kit of the invention may comprise a heavy chain variable region and/or a light chain variable region, wherein the heavy chain variable region is encoded by a nucleic acid sequence which is at least 70% identical to the nucleic acid sequence denoted by SEQ ID NO: 1 and wherein the light chain variable region is encoded by a nucleic acid sequence which is at least 70% identical to SEQ ID NO: 17. In some other embodiments, the antibody of the kit of the invention may comprise a heavy chain variable region comprising the amino acid sequence denoted by SEQ ID NO: 2 or a variant thereof and a light chain variable region comprising the amino acid sequence denoted by SEQ ID NO: 18 or a variant thereof. In some further embodiments, the antibody of the kit of the invention may comprise a Heavy chain Framework Region 1 (FR1) comprising the amino acid sequence denoted by SEQ ID NO: 4, a heavy chain FR2 comprising the amino acid sequence denoted by SEQ ID NO: 8 and a heavy chain FR3 comprising the amino acid sequence denoted by SEQ ID NO: 12 and a Light chain Framework Region 1 (FR1) comprising the amino acid sequence denoted by SEQ ID NO: 20, a Light chain FR2 comprising the amino acid sequence denoted by SEQ ID NO: 24 and a Light chain FR3 comprising the amino acid sequence denoted by SEQ ID NO: 28. In yet some further embodiments, the kits of the invention may comprise any antibody disclosed by the invention, as well as any antibody that recognizes and binds the epitope recognized by the antibodies of the invention. Still further, the kits of the invention may comprise any antibody that competes with any of the antibodies of the invention, specifically, any antibody that competes with an antibody comprising at least one of the CDRs as denoted by SEQ ID NO: 6, 10, 14, 22, 26 and 30, or any homologs or derivatives thereof. In yet some further embodiments, the kits of the invention may comprise any antibody that competes with an antibody comprising the variable heavy chain as denoted by SEQ ID NO: 2, or any homologs or derivatives thereof, and/or the variable light chain that comprises the amino acid sequence as denoted by SEQ ID NO: 18, or any homologs or derivatives thereof. In yet some embodiments, the kits of the present disclosure further encompasses any antibody the recognizes and binds the epitope of at least one of SEQ ID NO; 39, 48 and 49, or any combinations thereof. In more specific embodiments, the antibody of the kit of the invention may specifically recognize and bind at least one component of the T3SS of at least one bacterium. In some embodiments, the additional therapeutic agent may be an anti-bacterial agent. In certain embodiments, the kits and methods of the invention further include and use at least one additional therapeutic agent. Such additional agents of the kits of the invention may include at least one anti-bacterial agent, anti-fungal agent, growth factors, anti-inflammatory agents, vasopressor agents including but not limited to nitric oxide and calcium channel blockers, collagenase inhibitors, topical steroids, matrix metalloproteinase inhibitors, ascorbates, angiotensin II, angiotensin III, calreticulin, tetracyclines, fibronectin, collagen, thrombospondin, transforming growth factors (TGF), keratinocyte growth factor (KGF), fibroblast growth factor (FGF), insulin-like growth factors (IGFs), IGF binding proteins (IGFBPs), epidermal growth factor (EGF), platelet derived growth factor (PDGF), neu differentiation factor (NDF), hepatocyte growth factor (HGF), vascular endothelial growth factor (VEGF), heparin-binding EGF (HBEGF), thrombospondins, von Willebrand Factor-C, heparin and heparin sulfates, and hyaluronic acid. The term “antimicrobial agent” as used herein refers to any entity with antimicrobial activity (either bactericidal or bacteriostatic), i.e. the ability to inhibit the growth and/or kill bacterium, for example Gram negative bacteria. An antimicrobial agent may be any agent which results in inhibition of growth or reduction of viability of a bacteria by at least about 10%, 20%, 30% or at least about 40%, or at least about 50% or at least about 60% or at least about 70% or more than 70%, for example, 75%, 80%, 85%, 90%, 95%, 100% or any integer between 30% and 70% or more, as compared to in the absence of the antimicrobial agent. Stated another way, an antimicrobial agent is any agent which reduces a population of microbial cells, such as bacteria by at least about 30% or at least about 40%, or at least about 50% or at least about 60% or at least about 70% or more than 70%, or any integer between 30% and 70% as compared to in the absence of the antimicrobial agent. In one embodiment, an antimicrobial agent is an agent which specifically targets a bacteria cell. In another embodiment, an antimicrobial agent modifies (i.e. inhibits or activates or increases) a pathway which is specifically expressed in bacterial cells. An antimicrobial agent can include any chemical, peptide (i.e. an antimicrobial peptide), peptidomimetic, entity or moiety, or analogues of hybrids thereof, including without limitation synthetic and naturally occurring non-proteinaceous entities. In some embodiments, an antimicrobial agent is a small molecule having a chemical moiety. For example, chemical moieties include unsubstituted or substituted alkyl, aromatic or heterocyclyl moieties including macrolides, leptomycins and related natural products or analogues thereof. Antimicrobial agents can be any entity known to have a desired activity and/or property, or can be selected from a library of diverse compounds. In yet some further embodiments, such antibacterial agents may be antibiotic agents. Still further, in some embodiments such antibiotic agent may be at least one beta-lactam antibiotic agent. The term “β-lactam” or “β-lactam antibiotics” as used herein refers to any antibiotic agent which contains a b-lactam ring in its molecular structure. β-lactam antibiotics are a broad group of antibiotics that include different classes such as natural and semi-synthetic penicillins, clavulanic acid, carbapenems, penicillin derivatives (penams), cephalosporins (cephems), cephamycins and monobactams, that is, any antibiotic agent that contains a β-lactam ring in its molecular structure. They are the most widely-used group of antibiotics. While not true antibiotics, the β-lactamase inhibitors are often included in this group. β-lactam antibiotics are analogues of D-alanyl-D-alanine the terminal amino acid residues on the precursor NAM/NAG-peptide subunits of the nascent peptidoglycan layer. The structural similarity between β-lactam antibiotics and D-alanyl-D-alanine prevents the final crosslinking (transpeptidation) of the nascent peptidoglycan layer, disrupting cell wall synthesis. Under normal circumstances peptidoglycan precursors signal a reorganization of the bacterial cell wall and, as a consequence, trigger the activation of autolytic cell wall hydrolases. Inhibition of cross-linkage by β-lactams causes a buildup of peptidoglycan precursors, which triggers the digestion of existing peptidoglycan by autolytic hydrolases without the production of new peptidoglycan. As a result, the bactericidal action of B-lactam antibiotics is further enhanced. Generally, β-lactams are classified and grouped according to their core ring structures, where each group may be divided to different categories. The term “penam” is used to describe the core skeleton of a member of a penicillin antibiotic. i.e. a β-lactam containing a thiazolidine rings. Penicillins contain a β-lactam ring fused to a 5-membered ring, where one of the atoms in the ring is sulfur and the ring is fully saturated. Penicillins may include narrow spectrum penicillins, such as benzathine penicillin, benzylpenicillin (penicillin G), phenoxymethylpenicillin (penicillin V), procaine penicillin and oxacillin. Narrow spectrum penicillinase-resistant penicillins include methicillin, dicloxacillin and flucloxacillin. The narrow spectrum β-lactamase-resistant penicillins may include temocillin. The moderate spectrum penicillins include for example, amoxicillin and ampicillin. The broad spectrum penicillins include the co-amoxiclav (amoxicillin+clavulanic acid). Finally, the penicillin group also includes the extended spectrum penicillins, for example, azlocillin, carbenicillin, ticarcillin, mezlocillin and piperacillin. Other members of this class include pivampicillin, hetacillin, bacampicillin, metampicillin, talampicillin, epicillin, carbenicillin, carindacillin, tie arcillin, azlocillin, piperacillin, mezlocillin, mecillinam, pivmecillinam, sulbenicillin, clometocillin, procaine benzylpenicillin, azidocillin, penamecillin, propicillin, pheneticillin, cloxacillin and nafcillin. β-lactams containing pyrrolidine rings are named carbapenams. A carbapenam is a P-lactam compound that is a saturated carbapenem. They exist primarily as biosynthetic intermediates on the way to the carbapenem antibiotics. Carbapenems have a structure that renders them highly resistant to β-lactamases and therefore are considered as the broadest spectrum of β-lactam antibiotics. The carbapenems are structurally very similar to the penicillins, but the sulfur atom in position 1 of the structure has been replaced with a carbon atom, and hence the name of the group, the carbapenems. Carbapenem antibiotics were originally developed from thienamycin, a naturally-derived product of Streptomyces cattleya. The carbapenems group includes: biapenem, doripenem, ertapenem, imipenem, meropenem, panipenem and PZ-601. β-lactams containing 2, 3-dihydrothiazole rings are named penems. Penems are similar in structure to carbapenems. However, where penems have a sulfur, carbapenems have another carbon. There are no naturally occurring penems; all of them are synthetically made. An example for penems is faropenem. β-lactams containing 3, 6-dihydro-2H-1, 3-thiazine rings are named cephems. Cephems are a subgroup of β-lactam antibiotics and include cephalosporins and cephamycins. The cephalosporins are broad-spectrum, semisynthetic antibiotics, which share a nucleus of 7-aminocephalosporanic acid. First generation cephalosporins, also considered as the moderate spectrum includes cephalexin, cephalothin and cefazolin. Second generation cephalosporins that are considered as having moderate spectrum with anti-Haemophilus activity may include cefaclor, cefuroxime and cefamandole. Second generation cephamycins that exhibit moderate spectrum with anti-anaerobic activity include cefotetan and cefoxitin. Third generation cephalosporins considered as having broad spectrum of activity includes cefotaxime and cefpodoxime. Finally, the fourth generation cephalosporins considered as broad spectrum with enhanced activity against Gram positive bacteria and β-lactamase stability include the cefepime and cefpirome. The cephalosporin class may further include: cefadroxil, cefixime, cefprozil, cephalexin, cephalothin, cefuroxime, cefamandole, cefepime and cefpirome. Cephamycins are very similar to cephalosporins and are sometimes classified as cephalosporins. Like cephalosporins, cephamycins are based upon the cephem nucleus. Cephamycins were originally produced by Streptomyces, but synthetic ones have been produced as well. Cephamycins possess a methoxy group at the 7-alpha position and include: cefoxitin, cefotetan, cefmetazole and flomoxef. β-lactams containing 1, 2, 3, 4-tetrahydropyridine rings are named carbacephems. Carbacephems are synthetically made antibiotics, based on the structure of cephalosporin, a cephem. Carbacephems are similar to cephems but with a carbon substituted for the sulfur. An example of carbacephems is loracarbef. Monobactams are β-lactam compounds wherein the β-lactam ring is alone and not fused to another ring (in contrast to most other B-lactams, which have two rings). They work only against Gram negative bacteria. Other examples of monobactams are tigemonam, nocardicin A and tabtoxin. β-lactams containing 3, 6-dihydro-2H—I, 3-oxazine rings are named oxacephems or clavams. Oxacephems are molecules similar to cephems, but with oxygen substituting for the sulfur. Thus, they are also known as oxapenams. An example for oxapenams is clavulanic acid. They are synthetically made compounds and have not been discovered in nature. Other examples of oxacephems include moxalactam and flomoxef. Another group of β-lactam antibiotics is the β-lactamase inhibitors, for example, clavulanic acid. Although they exhibit negligible antimicrobial activity, they contain the β-lactam ring. Their sole purpose is to prevent the inactivation of β-lactam antibiotics by binding the β-lactamases, and, as such, they are co-administered with P-lactam antibiotics. β-lactamase inhibitors in clinical use include clavulanic acid and its potassium salt (usually combined with amoxicillin or ticarciiiin), sulbactam and tazobactam. In some further embodiments, the antibody of the kits of the invention recognizes T3SS components of MDR bacteria. In some particular embodiments, such bacteria may be a gram negative bacteria. In some specific embodiments, the MDR bacterium may be at least one of EPEC and EHEC. In some more specific embodiments, the bacterium may be EPEC. In some embodiments, the antibody of the kit of the invention modulates the immune system of a subject. In yet some further embodiments, the antibody induces at least one of a humoral response and immunological memory in a subject. In some further embodiments, the antibody of the kit of the invention may bind the EspB protein expressed by any of the bacteria disclosed above. In other embodiments, the antibody of the kit of the invention may interfere with or inhibit at least one of the assembly and the secretion of at least one component of the T3SS. In yet some further alternative embodiments, the antibody of the invention does not interfere with or inhibit the assembly and the secretion the EspB protein and the EspD protein. In more specific embodiments, the antibody of the kit of the invention may interfere with the assembly and/or the secretion of the EspB protein and the EspD protein of the bacterium. In yet some further embodiments, the kits of the invention may be for use in a method of treating, preventing, ameliorating, reducing or delaying the onset of an infection by at least one bacterium expressing at least one T3SS. In providing an antibody that specifically recognizes and binds a component of T3SS of MDR bacteria, the invention may also provide the ability of detecting bacteria that comprise such elements. As such, the invention further provide specific diagnostic application for the antibodies of the invention. Thus, in yet another aspect, the invention provides a diagnostic method for detecting at least one bacterium expressing a T3SS in at least one biological sample, the method comprising the following steps. The first step (a), involves contacting the at least one biological sample with at least one antibody or with any antigen-binding fragment thereof, or with any matrix, nano- or micro-particles thereof. More specifically, the antibody used by the diagnostic methods of the invention comprises at least one heavy chain CDRH1 comprising the amino acid sequence GFTFSHYA, as denoted by SEQ ID NO: 6, or any variants, homologs or derivatives thereof, CDRH2 comprising the amino acid sequence INSNGDST, as denoted by SEQ ID NO: 10, or any variants, homologs or derivatives thereof, CDRH3 comprising the amino acid sequence ARDRRAGYFDYW, as denoted by SEQ ID NO: 14, or any variants, homologs or derivatives thereof, and at least one light chain CDRL1 comprising the amino acid sequence RDNIGKNY as denoted by SEQ ID NO: 22, or any homologs or derivatives thereof, CDRL2 comprising the amino acid sequence RNN as denoted by SEQ ID NO: 26, or any homologs or derivatives thereof, and CDRL3 comprising the amino acid sequence SAWDTSLNA as denoted by SEQ ID NO: 30, or any homologs or derivatives thereof, or any derivative, variant and biosimilar thereof, or any antigen-binding fragment thereof. In some embodiments, the antibody of the kits disclosed herein, is directly or indirectly associated with at least one detectable moiety and/or solid support. The second step (b), involves determining the presence of the detectable moiety. It should be noted that the presence of the detectable moiety indicates the presence of an at least one bacterium expressing a T3SS in said biological sample. Thus, determining the presence of at least one bacterium expressing a T3SS in said biological sample, if said detectable moiety is detected. In certain embodiments, the bacterium detectable by the diagnostic methods may be at least one MDR bacterium. In some specific embodiments, such MDR bacterium may be at least one of EPEC and EHEC. In some more specific embodiments, the bacterium may be EPEC. In some embodiments, the at least one bacterium referred herein may be gram-negative bacteria. In yet another embodiment, the antibody suitable for the methods of the invention may bind the EspB protein expressed by the bacterium. In some embodiments, the antibody modulates the immune system of a subject. In some embodiments, the antibody induces opsonization and phagocytic clearance in the subject. In yet some further embodiments, the antibody of the present disclosure induces at least one of a humoral response and immunological memory in a subject. In some further embodiments, the antibody used by the diagnostic methods of the invention may interfere with or inhibit at least one of the assembly and the secretion of at least one component of the T3SS. In certain embodiments, the antibody may interfere with the assembly and/or the secretion of the EspB protein and the EspD protein of the bacterium. In yet some further alternative embodiments, the antibody of the invention does not interfere with or inhibit the assembly and the secretion the EspB protein and the EspD protein. In yet an alternative embodiment, the diagnostic methods of the invention may be specifically applicable for detecting an infection by of at least one T3SS expressing bacteria in a mammalian subject, specifically, any mammalian subject as defined by the present disclosure. In yet some further embodiments, the diagnostic methods and kits may be applicable for any sample. The terms “sample”, “test sample” and “specimen”, “biological sample” are used interchangeably in the present specification and claims and are used in its broadest sense. They are meant to include both biological and environmental samples and may include an exemplar of synthetic origin. This term refers to any media that may contain the T3SS expressing bacteria and may include body fluids (urine, blood, milk, cerebrospinal fluid, rinse fluid obtained from wash of body cavities, phlegm, pus), samples taken from various body regions (throat, vagina, ear, eye, skin, sores), food products (both solids and fluids) and swabs taken from medicinal instruments, apparatus, materials), as well as substances in which controlled chemical reactions are being carried out. More specifically, according to certain embodiments, the method of the invention uses any appropriate biological sample. The term “biological sample” in the present specification and claims is meant to include samples obtained from any subject or environmental sources, for example, a mammal subject. It should be recognized that in certain embodiments a biological sample may be for example, blood cells, blood, serum, plasma, bone marrow, lymph fluid, urine, sputum, saliva, feces, semen, spinal fluid or CSF, the external secretions of the skin, respiratory, intestinal, and genitourinary tracts, tears, milk, any human organ or tissue, any sample obtained by lavage, optionally of the breast ducal system, plural effusion, sample of in vitro or ex vivo cell culture and cell culture constituents. In certain embodiment, the biological sample suitable for the method of the invention may be any one of serum, whole blood sample, urine, saliva, or any fraction or preparation thereof. In some embodiments, the sample applicable in the methods, and kits of the invention may be either as naturally obtained from the tested subject or manipulated and prepared. In some embodiments, the body fluid samples may be concentrated samples. In yet some further embodiments, the serum samples may be diluted and as such, different sera concentrations may be used. In some further embodiments the serum concentration may range between about 0.01% and 100%, More specifically, 0.01%, 0.02%, 0.03%, 0.04%, 0.05%, 0.06%, 0.07%, 0.08%, 0.09%, 0.1%, 0.2%, 0.2%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19% or 20%, 25%, 30%, 35%, 40%, 45%, 50%, 55%, 60%, 65%, 70%, 75%, 80%, 85, 90%, 95%, 100% or more. In more specific embodiment, the sample concentration may range between about 1% to about 20%, in yet some further particular embodiments, the sample concentration of the sample may be 5%. It should be further noted that in some embodiments, the diagnostic methods of the invention may be also applicable for environmental samples. Environmental samples include environmental material such as surface matter, earth, soil, water, air and industrial samples, as well as samples obtained from food and dairy processing instruments, apparatus, equipment, utensils, disposable and non-disposable items. These examples are not to be construed as limiting the sample types applicable to the present invention. The sample may be any media, specifically, a liquid media that may contain the T3SS expressing bacteria. Typically substances and samples or specimens that are a priori not liquid may be contacted with a liquid media which is contacted with the antibody composition of the invention. More specifically, by the term “food”, it is referred to any substance consumed, usually of plant or animal origin. Some non-limiting examples of animals used for feeding are cows, pigs, poultry, etc. The term food also comprises products derived from animals, such as, but not limited to, milk and food products derived from milk, eggs, meat, etc. In some specific embodiments, the present invention encompasses samples of a substance, which is used as a drink. A drink or beverage is a liquid which is specifically prepared for human consumption. Non limiting examples of drinks include, but are not limited to water, milk, alcoholic and non-alcoholic beverages, soft drinks, fruit extracts, etc. It should be understood that the diagnostic methods disclosed by the invention may be further used for monitoring subjects treated with any therapeutic compound, and/or with the antibodies of the invention. More specifically, the diagnostic methods of the invention may be further used form monitoring the extent of infection (or bacterial load) in the treated subject. For such monitoring purpose, the steps of the methods of the invention may be repeated at least one further time for at least one further sample obtained from the subject. In some embodiments, the sample is obtained in another time point and is therefore considered herein as a temporally separated sample. As indicated above, in accordance with some embodiments of the invention, in order to assess the patient condition, or monitor the disease progression, as well as responsiveness to a certain treatment, at least two “temporally-separated” test samples must be collected from the examined patient and compared thereafter in order to obtain the rate of change in the amount of bacteria between said samples, as reflected by the amount of the detectable moiety associated directly or indirectly with the antibody of the invention, as determined in the sample. In practice, to detect a change in at least one of these parameters between said samples, at least two “temporally-separated” test samples and preferably more must be collected from the patient. This period of time, also referred to as “time interval”, or the difference between time points (wherein each time point is the time when a specific sample was collected) may be any period deemed appropriate by medical staff and modified as needed according to the specific requirements of the patient and the clinical state he or she may be in. For example, this interval may be at least one day, at least three days, at least three days, at least one week, at least two weeks, at least three weeks, at least one month, at least two months, at least three months, at least four months, at least five months, at least one year, or even more. When calculating the rate of change, one may use any two samples collected at different time points from the patient. To ensure more reliable results and reduce statistical deviations to a minimum, averaging the calculated rates of several sample pairs is preferable. A calculated or average value of a negative rate of change in bacterial load, as reflected by the amount of the detectable moiety associated directly or indirectly with the antibody of the invention, indicates that said subject exhibits a beneficial response to said treatment; thereby monitoring the efficacy of a treatment with the antibody of the invention and/or with any additional therapeutic agent and the disease progression. The number of samples collected and used for evaluation of the subject may change according to the frequency with which they are collected. For example, the samples may be collected at least every day, every two days, every four days, every week, every two weeks, every three weeks, every month, every two months, every three months every four months, every 5 months, every 6 months, every 7 months, every 8 months, every 9 months, every 10 months, every 11 months, every year or even more. For the described diagnostic applications, the invention further provides in further aspects thereof, diagnostic kits. Thus, in yet another aspect, the invention provides a diagnostic kit comprising: (a) at least one antibody or any antigen-binding fragment thereof, or a any matrix, nano- or micro-particles thereof, the antibody comprising at least one heavy chain CDRH1 comprising the amino acid sequence GFTFSHYA, as denoted by SEQ ID NO: 6, CDRH2 comprising the amino acid sequence INSNGDST, as denoted by SEQ ID NO: 10, CDRH3 comprising the amino acid sequence ARDRRAGYFDYW, as denoted by SEQ ID NO: 14, and at least one light chain CDRL1 comprising the amino acid sequence RDNIGKNY as denoted by SEQ ID NO: 22, CDRL2 comprising the amino acid sequence RNN as denoted by SEQ ID NO: 26, and CDRL3 comprising the amino acid sequence SAWDTSLNA as denoted by SEQ ID NO: 30, or any derivative, variant and biosimilar thereof, wherein the antibody is directly or indirectly associated with at least one detectable moiety and/or a solid support; the kit optionally further comprises at least one of: (b) means for detecting the presence of the at least one detectable moiety; (c) at least one control sample and/or control standard value; (d) instructions for use of the kit. As indicated herein, for diagnostic and monitoring purposes, the antibody of the invention may be directly or indirectly associated with a detectable moiety that facilitates quantification and identification thereof. In some further embodiments, the detectable moiety associated directly or indirectly with the antibody of the invention, may refer to any chemical moiety that can be used to provide a detectable signal, and that can be attached to a nucleic acid or protein via a covalent bond or noncovalent interaction (e.g., through ionic or hydrogen bonding, or via immobilization, adsorption, or the like). Labels generally provide signals detectable by at least one of fluorescence, chemiluminescence, radioactivity, colorimetry, mass spectrometry, X-ray diffraction or absorption, magnetism, enzymatic activity, electrochemical active compounds, or the like. In some specific embodiments, the detectable moiety may be at least one of conductive, electrochemical, fluorescent, chemiluminescent, enzymatic, radioactive, magnetic, metal, and colorimetric label, or any combinations thereof. Examples of labels useful in connection with the invention, include, but are not limited to at least one of haptens, enzymes, enzyme substrates, coenzymes, enzyme inhibitors, fluorophores, quenchers, chromophores, magnetic particles or beads, redox sensitive moieties (e.g., electrochemically active moieties), luminescent markers, radioisotopes (including radionucleotides), conductive materials, or electrochemical materials that in some embodiments may be suitable for electrochemical detection, specifically, nano- and micro-sized materials, such as gold nanoparticles (GNPs), latex, carbon nanotubes (CNTs), graphene (GR), magnetic particles (MBs), quantum dots (QDs) and conductive polymers, biobarcodes and members of binding pairs. More specific examples include at least one of fluorescein, phycobiliprotein, tetraethyl rhodamine, and beta-galactosidase. Binding pairs may include biotin/Strepavidin, biotin/avidin, biotin/neutravidin, biotin/captavidin, GST/glutathione, maltose binding protein/maltose, calmodulin binding protein/calmodulin, enzyme-enzyme substrate, receptor-ligand binding pairs, and analogs and mutants of the binding pairs. It should be appreciated that the use of tags for labeling directly or indirectly the antibody of the invention, is also encompassed by the invention. Non-limiting examples for such tag may include His-tag, Flag, HA, myc and the like. It should be further appreciated that the detectable moieties disclosed herein are applicable for any aspect of the invention. In certain embodiments, the diagnostic kit of the invention may be applicable for detecting the presence of at least one bacterium expressing at least one T3SS in at least one biological sample. In some embodiments, the bacterium referred herein may be at least one MDR bacterium. In more other embodiments, the at least one bacterium may be a gram negative bacteria. In another embodiment, the MDR bacterium may be at least one of EPEC and EHEC, as defined herein before. In some more specific embodiments, the bacterium may be EPEC. In some further embodiments, the antibody of the diagnostic kit of the invention may bind the EspB protein expressed by the bacterium. In some embodiments, the antibody modulates the immune system of a subject. In some embodiments, the antibody induces opsonization and phagocytic clearance in the subject. In yet some further embodiments, the antibody of the present disclosure induces at least one of a humoral response and immunological memory in a subject. In some embodiments, the diagnostic kit of the invention may be for use in detecting infection of a mammalian subject by at least one T3SS expressing bacteria. A further aspect of the invention provides a method for modulating the immune system of a subject infected by at least one T3SS expressing bacteria. The method comprising the step of administering to the subject a therapeutically effective amount of at least one monoclonal antibody or any antigen-binding fragment thereof, or any matrix, nano- or micro-particles thereof, wherein the antibody comprises at least one heavy chain CDRH1 comprising the amino acid sequence GFTFSHYA, as denoted by SEQ ID NO: 6, CDRH2 comprising the amino acid sequence INSNGDST, as denoted by SEQ ID NO: 10, CDRH3 comprising the amino acid sequence ARDRRAGYFDYW, as denoted by SEQ ID NO: 14, and at least one light chain CDRL1 comprising the amino acid sequence RDNIGKNY as denoted by SEQ ID NO: 22, CDRL2 comprising the amino acid sequence RNN as denoted by SEQ ID NO: 26, and CDRL3 comprising the amino acid sequence SAWDTSLNA as denoted by SEQ ID NO: 30, or any derivative, variant and biosimilar thereof. In some embodiments, the antibody suitable for the method of the invention may be as detailed above in the present disclosure. In some further embodiments, the subject administered with the antibody of the invention may suffer with at least one of transient enteritis or colitis, cholecystitis, bacteremia, cholangitis, urinary tract infection (UTI), traveler's diarrhea, neonatal meningitis and pneumonia, or any conditions, symptoms or effects associated therewith. In some embodiments, the invention may further provide a method for inhibiting at least one of the assembly and/or the secretion of at least one component of the T3SS system of at least one bacterium in a subject in need thereof, using the antibody of the invention. As used in this specification and the appended claims, the singular forms “a”, “an”, and “the” include plural references unless the context clearly dictates otherwise. Thus for example, references to “a method” includes one or more methods, and/or steps of the type described herein and/or which will become apparent to those persons skilled in the art upon reading this disclosure and so forth. Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. More specifically, the terms “comprises”, “comprising”, “includes”, “including”, “having” and their conjugates mean “including but not limited to”. This term encompasses the terms “consisting of” and “consisting essentially of”. The phrase “consisting essentially of” means that the composition or method may include additional ingredients and/or steps, but only if the additional ingredients and/or steps do not materially alter the basic and novel characteristics of the claimed composition or method. The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. As used herein the term “about” refers to ±10%. It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between. The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric. The examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention. It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements. Various embodiments and aspects of the present invention as delineated hereinabove and as claimed in the claims section below find experimental support in the following examples. Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

### EXAMPLES

### Experimental Procedures

### Bacterial Strains

Wild-type (WT) EPEC 0127:H6 strain E2348/69 [streptomycin-resistant] and EPEC null mutants (AescN, AespB, ΔespD) were used to purify EspB, to evaluate mAb-EspB-B7 binding, and to assess T3SS and translocation activities. WT and T3SS-mutant strains of Citrobacter rodentium DBS100, enterohemorrhagic E. coli (EHEC), and Salmonella enterica serovar Typhimurium were used to assess antibody specificity. Antibiotics were used at the following concentrations: streptomycin (50 μg/mL), ampicillin (100 μg/mL), chloramphenicol (30 μg/mL), and nalidixic acid (50 μg/mL).

### Expression and Purification of Recombinant Espb

EPEC 0127:H6 strain E2348/69 deleted for the espB gene (ΔespB) was transformed with a bacterial expression vector encoding His-tagged EspB (EspB-His) and grown overnight in Luria-Bertani (LB) broth supplemented with the appropriate antibiotics. The following steps of the expression and purification of EspB are described herein. More specifically, the overnight culture was diluted 1:50 and grown for 3 hr under T3SS-inducing conditions (pre-heated Dulbecco's modified Eagle's medium [DMEM] in a tissue culture incubator with 5% CO2, statically). These conditions induce the secretion of EspB into the extracellular environment. Next, 0.5 mM isopropyl-β-d-thiogalactopyranoside (IPTG) was added, and the culture was grown for an additional 4 hr. The culture was centrifuged for 30 min at 12000×g, and the supernatant containing the secreted EspB-His was collected and supplemented with protease inhibitor cocktail of 200 mM phenylmethylsulfonyl fluoride (PMSF) and 1 μM benzamidine. The supernatant was then loaded on a His-Trap HP 1-mL column (GE Healthcare), washed with 50 mM imidazole, and eluted with 500 mM imidazole, according to the manufacturer's protocol. The elution fractions were analyzed by SDS-PAGE and Coomassie staining to identify the fractions that contain the purified protein. The recovered protein was further purified by gel filtration chromatography using a Superose 12 10/300 GL column (GE Healthcare). The peak fractions were collected, frozen in liquid nitrogen and stored at −80° C.

### Bio-Panning Cycles Output Screening by Phage-ELISA

E. coli TG-1 cultures were separately infected by the phage output of the third and fourth bio-panning cycles. 190 infected TG-1 single colonies were randomly picked for evaluation by phage-ELISA, and phages were prepared from each colony. Phages binding affinity to EspB or control antigen was evaluated by ELISA and detected using anti-phage HRP conjugate. Phages from the 3^{rd} cycle output were tested against EspB and human TNF-α, and phages from the 4 ^{th} cycle output were tested against EspB and BSA.

### Phage Panning, mAb-EspB-B7 Expression and Purification

A human synthetic-phage library, displaying single-chain variable fragment (scFv), was used to isolate antibodies targeting EspB as described previously. mAb-EspB-B7 V_{H} and V_{L} were cloned in mammalian expression vectors (pcDNA3.4H and pcDNA3.4 L encoding the IgG1 heavy and lambda light chain constant regions) by Gibson cloning. The cloned vectors were transformed into E. coli competent cells (XL-1 blue) and were purified using plasmid purification kit (Invitrogen). The vectors were co-transfected into Expi293 expression system (Gibco) according to the manufacturer's instructions. Transfected Expi293 cells were harvested by centrifugation at 2000×g for 10 min at 4° C. and conditioned medium was applied to MabSelect affinity column (GE Healthcare) according to the manufacturer's instructions.

### ELISA Binding Assays

### mAb-B7 Binding Affinity to EspB

A 96 well ELISA plate was coated with 50 μl of 5 μg/ml of EspB or no antigen for control and incubated over night at 4° C. Then, the plate was blocked with 300 μl/well of 3% [w/v]skim milk in PBS for 1 h at 37° C. and was washed with PBS-T in a plate washer (microplate washer, 405LSRS, BioTek). mAb-B7 was applied to the first line of the plate, followed by serial dilution throughout the plate. The plate was incubated for 1 h at room temperature and washed. Next, goat anti-human H+L HRP-conjugated secondary antibody was added to the plate for 1 h at room temperature. Finally, 50 μl/well of TMB were applied. Reaction was stopped by adding 50 μl/well of 1M H2SO4.

### ELISA Binding Assay of Epitope Peptides

A 96 well ELISA plate was coated with 50 μl of serial dilution of Peptide #49 (as denoted by SEQ ID NO: 33), peptide #49-scrambled (as denoted by SEQ ID NO: 34), peptide #50 (as denoted by SEQ ID NO: 35), peptide #50-scrambled (as denoted by SEQ ID NO: 36) or #49+50 (as denoted by SEQ ID NO: 37) and incubated over night at 4° C. Peptide #78 (as denoted by SEQ ID NO: 38) or intact EspB protein were used as controls. Then, the plate was blocked with 300 μl/well of 3% [w/v]skim milk in PBS for 1 h at 37° C. and was washed with PBS-T in a plate washer (microplate washer, 405LSRS, BioTek). mAb-B7 (20 nM) was applied to the plate for 1 h at room temperature and washed. Next, goat anti-human H+L HRP-conjugated secondary antibody was added to the plate for 1 h at room temperature. Finally, 50 μl/well of TMB were applied. Reaction was stopped by adding 50 μl/well of 1M H2SO4. Plate absorbance at A₄₅₀ₙₘ was read using microplate reader (Epoch, BioTek).

### Enzyme-Linked Immunosorbent Assay (ELISA)

For all ELISA experiments, 96-well ELISA plates were coated with 5 μg/mL of target antigen in PBS and incubated overnight at 4° C. Blocking, washing and detection steps were carried out as described previously. More specifically, EspB coated 96-well plates were blocked with 300 μL/well of 3% [w/v] skim milk in PBS for 1 hr at 37° C. and washed with PBS. mAb-EspB-B7 in blocking solution was added to the first line of the plate and serially diluted throughout the plate. The plate was incubated for 1 hr at room temperature, washed, and incubated with goat anti-human H+L HRP-conjugated secondary antibody in 0.05% PBST (Jackson ImmunoResearch) for 1 hr at room temperature. Plates were then washed and signal was developed using 3,3′,5,5′-tetramethylbenzidine (TMB). The reactions were quenched by 1 M H₂SO₄ and absorbance was measured at optical density (OD) of 450 nm (Epoch, BioTek). ELISA assays to test mAb-EspB-B7 binding in various conditions were carried out using similar protocol as described above with the following modifications: (i) for binding under various pH conditions, mAb-EspB-B7 was incubated in 0.1 M citric acid buffer pH 7.4, 7.0, 6.6, 5.6, and 4.6 during the binding step; (ii) for binding at various salt concentrations, mAb-EspB-B7 was incubated in 45.6 nM, 68.5 nM, 137 nM, 274 nM, and 411 nM NaCl; and (iii) for assessment of the serum effect on mAb-EspB-B7 binding, the antibody was incubated in 10% goat or horse serum with 1% Tween 20 and 1% human serum during the binding step. Competitive ELISA with peptides was carried out as follows: A 96-well ELISA plate (I) and a 96-well inert Bradford plate (II) were used for each of the peptides examined. The respective scrambled peptides (carrying the same amino acid compositions in a scrambled order), full-length EspB and peptide #78 (SEQ ID NO: 38) were used as positive and negative controls, respectively. Plate I was coated with 3 μg/ml EspB or PBS and incubated overnight at 4° C. Blocking of plate I was performed as described above. mAb-EspB-B7 (15 nM) was pre-incubated with serially diluted concentrations of peptides, starting at 15 μg/mL for 1 hr at room temperature, transferred to plate I, and incubated for 1 hr at room temperature. The remaining steps were performed as described above for regular ELISA.

### Competitive ELISA

Competitive ELISA was performed between intact EspB protein and EspB derived peptides that were identified as part of the mAb epitope. 96 well ELISA plate was coated with 50 μl of 3 μg/ml of EspB and incubated over night at 4° C. Then, the plate was blocked with 300 μl/well of 3% [w/v] skim milk in PBS for 1 h at 37° C. and was washed with PBS-T in a plate washer. Peptide #49, peptide #49-scrambled, peptide #50, peptide #50-scrambled or peptide #49+50 were incubated for 1 h at room temperature with mAb-B7 at constant concentration of 15 nM (around mAb-B7 K_{D}). Peptide #78 or no peptide were used as negative controls. Next, the mixture of mAb-B7 and the different peptides was added to the plate. The peptides were serially diluted through the plate, while mAb-B7 concentration remains constant and incubated for 1 h at room temperature. Then, the plate was treated with goat anti-human H+L HRP-conjugated secondary antibody as described above.

### Biacore

The binding kinetics of mAb-B7 to EspB were measured on a Biacore T200 instrument at 25° C. EspB protein was covalently coupled to CMS sensor chip surface. Measurements were performed in a buffer containing PBS, surfactant P20, HEPES 10 mM, NaCl 150 mM, EDTA 3 mM, pH 7.4. 200 μl of various concentrations of mAb-B7 ranging from 10-90 nM were injected at a flow rate of 30 μL/minute. For regeneration of the sensor chip surface NaOH 5 mM was applied. The kinetic data were fitted and analyzed with the Biacore T200 Evaluation Software 3.0 and respective rate constants (kₒₙ, k_{off}) and K_{D} values were calculated. Infliximab antibody was used as negative control.

### Western Blot

EPEC WT, ΔescN and ΔespB+pAS10−EspB—His strains were grown under T3SS-inducing conditions (statically, in DMEM medium with 5% CO2) for 6 h. The bacteria pellets and supernatants were separated and analyzed using SDS-PAGE and Western blotting with mAb-B7. EPEC and C. rodentium bacteria were grown under T3SS-inducing conditions. Bacterial supernatants were analyzed by SDS-PAGE and immunoblotting by incubating the membrane with mAb-B7 and an anti-human IgG HRP conjugated antibody.

EPEC, EHEC, C. rodentium and Salmonella bacteria were grown under T3SS-inducing conditions. Mutated strains of EPEC, EHEC and C. rodentium are ΔescN, the mutated strain of Salmonella is ΔinvA (deletion in the structural T3SS gene). Bacteria were centrifuged and supernatants were collected and analyzed by SDS-PAGE and immunoblotting with mAb-B7 and an anti-human IgG HRP-conjugated antibody.

### Immunoblotting

Samples were subject to immunoblotting as described previously. Samples were subjected to SDS-PAGE and transferred to nitrocellulose membranes (pore size: 0.45 μm, Bio-Rad) or polyvinylidene difluoride (PVDF, Mercury, Millipore). The blots were blocked for 1 hr with 5% (w/v) skim milk-PBST (0.1% Tween in phosphate-buffered saline), incubated with the primary antibody (diluted in 5% skim milk-PBST for 1 hr at room temperature or overnight at 4° C.), washed, and then incubated with the secondary antibody (diluted in 5% skim milk-PBST, for 1 hr at room temperature). Chemi-luminescence was detected with EZ-ECL reagents (Biological Industries). The following primary antibodies were used: mAb-EspB-B7, diluted 1:1000; mouse anti-EspB (a gift from Prof. Finlay, University of British Columbia), diluted 1:1000; mouse anti-His (Pierce), diluted 1:2000; mouse anti-JNK (BD Pharmingen), diluted 1:1000 in TBS; and mouse anti-actin (MPBio), diluted 1:10,000. The following secondary antibodies were used: horseradish peroxidase-conjugated (HRP)-goat anti-mouse (Abcam Inc.) and HRP-conjugated goat anti-human (Abcam Inc) antibodies.

### Flow Cytometry

WT EPEC, ΔescN, ΔespB and ΔespB+pSA10−EspB—His bacteria were grown overnight in LB with appropriate antibiotics. The cultures were diluted 1:50 in DMEM and grown in a tissue culture incubator (with 5% CO2) for 3 h, to induce T3SS formation. 1×10⁷ bacteria were plated on a 96U plate and centrifuged at 800 g for 5 min and the supernatant was removed. Bacteria were incubated with mAb-B7 (1:100) for 1 h at room temperature. Samples were washed with phosphate-buffered saline (PBS) and stained using Alexa Fluor 488 goat anti-human IgG antibody (Jackson ImmunoResearch) for 30 min Samples were washed and resuspended in PBS for analysis. Flow cytometry analysis was performed on the Gallios instrument (Beckman Coulter). Further analysis was performed using Kaluza software (Beckman Coulter).

### Flow Cytometry

EPEC bacteria were grown overnight in LB with the appropriate antibiotics. The cultures were diluted 1:40 and grown under T3SS-inducing conditions for 3 hr. Thereafter, 1×10⁷ bacteria were plated in a 96-U shape well plate and centrifuged at 800×g for 5 min, and the supernatants were removed. Bacteria were incubated with primary antibody (mAb-EspB-B7, 1:100) for 1 hr at room temperature, washed with PBS, and stained using Alexa Fluor 488 goat anti-human IgG secondary antibody (Jackson ImmunoResearch) for 30 min. Samples were washed and resuspended in PBS for analysis. Flow cytometry analysis was performed on Gallios (Beckman Coulter) equipped with 488 nm, 405 nm and 638 nm lasers and a switchable 561 nm laser. Data analysis was performed with Kaluza software (Beckman Coulter).

### PEPSTAR PEPTIDE MICROARRAYS

Pepstar peptide microarrays were provided by JPT Peptide Technologies GmbH. The peptides on the arrays represent a scan of 15-mer peptides (78 linear peptides and 78 cyclic peptides) derived from EspB protein that overlap by 11 amino acids. Each microarray included three identical subarrays as technical triplicates. As a positive control, whole EspB was spotted in the array, while BSA served as negative control. The binding of mAb-B7 to the peptide array was carried out according to the manufacturer's instructions (www.jpt.com), with the following modifications: (i) mAb-B7 in TBS-Tween (TBST; 0.1% vol/vol) was incubated with the peptide microarray at a concentration of 20 μg/mL for 2 h at room temperature; (ii) peptide microarray slides were washed five times with TBST, incubated with Alexa Fluor 647-affinipure mouse anti human IgG (Jackson ImmunoResearch) for 45 min at room temperature, and subsequently washed five times with TBST and then five times with ddH2O; and (iii) slides were dried by spinning down. Fluorescent signals were acquired with a GenePix 4000B scanner (Molecular Devices) at a resolution with a pixel size of 10 μm, at 350 gain. Image analysis was carried out with Genepix Pro 6.0 analysis software (Molecular Devices). Signals obtained were normalized and plotted to reflect the relative intensities of the fluorescence signals.

### SDS Gel Analysis

ΔespB bacteria were grown under T3SS-inducing conditions, bacterial supernatants were collected and loaded onto Ni-beads column and with or without mAb-B7 (200 nM). The initial supernatants, the flow-through and the elution samples were on resolved by SDS-PAGE and immunoblotted with anti-His or anti-EspB polyclonal antibodies.

### Infectivity of HeLa Cells

EPEC WT, ΔescN, ΔespB and ΔespB+pEspB bacteria were grown under T3SS-inducing conditions before infecting HeLa cells in the presence or the absence of 400 nM mAb-B7. The cells were then washed, collected and lysed. Samples were loaded on SDS-PAGE and analyzed by western immunoblotting with anti-JNK antibody.

### Surface Plasmon Resonance (SPR)

Association and dissociation of the EspB-mAb-EspB-B7 complex was monitored by SPR with a Biacore 200 apparatus (GE Healthcare Life Sciences) with EspB immobilized on a CMS chip (GE Healthcare Life Sciences). SPR experiments were conducted according to the manufacturer protocols Immobilization of EspB on CMS chip was carried out by amine coupling chemistry using the following protocol at a flow rate of 10 μL/min and with 20 mM phosphate buffer with 0.15 M NaCl, and 0.005% Tween 20 at pH 5.91 as a running buffer. The chip was first activated by injecting a freshly prepared mixture of 50 mM N-hydroxysuccinimide and 195 mM 1-ethyl-3-β-dimethylaminopropyl) carbodiimide for 7.5 mM, then EspB (2.5 μg/mL in PBS buffer containing surfactant P20, 10 mM HEPES pH 7.4, 150 mM NaCl, and 3 mM EDTA) was injected for 5 mM to reach 120 resonance units (RU), and finally the remaining activated carboxylic groups were blocked by injecting 1 M ethanolamine hydrochloride, pH 8.6, for 5 min. The association of mAb-EspB-B7 with EspB was monitored by injecting different concentrations of mAb-EspB-B7 for 4 min at a flow rate of 30 μL/min, and the dissociation was monitored at the end of the antibody injection. To regenerate the chip, 5 mM NaOH solution was used. Data analysis was carried out by fitting the sensorgrams to the steady state model (T200 evaluation software).

### In Vitro Type 3 Secretion Assay

In vitro T3SS assay was carried out as described previously for EPEC and Salmonella. EPEC strains were grown overnight in LB supplemented with the appropriate antibiotics in a shaker at 37° C. The cultures were diluted 1:40 into pre-heated DMEM (Biological Industries) and grown statically for 6 hr in a tissue culture incubator (with 5% CO2), to an OD of 0.7 at 600 nm (OD600). These conditions simulate host environment and induce T3SS expression. The cultures were then centrifuged at 20000×g for 5 min to separate the bacterial pellets from the supernatants; the pellets were dissolved in SDS-PAGE sample buffer, and the supernatants were collected and filtered through a 0.22-μm filter (Millipore). The supernatants were then precipitated with 10% (v/v) trichloroacetic acid (TCA) overnight at 4° C. to concentrate proteins secreted into the culture medium. The volume of the supernatants was normalized to the bacterial cultures at OD600 to ensure equal loading of the samples. The samples were then centrifuged at 18000×g for 30 min at 4° C., the precipitates of the secreted proteins were dissolved in SDS-PAGE sample buffer, and the residual TCA was neutralized with saturated Tris. The T3SS activity of C. rodentium was determined similarly to that described for EPEC. For EHEC, the inventors cultured double the amount of EPEC (8 mL cultures instead of 4 mL) due to lower amounts of secreted proteins of EHEC relative to EPEC.

### Co-Elution of EspB and EspD35-his by Nickel Affinity Chromatography

Co-elution assays were performed as previously described [8]. EPEC ΔespD in the presence or the absence of an EspD-³⁵His expression vector, was grown under T3SS-inducing conditions for 7 hr (0.5 mM IPTG was added after 3 hr to induce protein expression). To evaluate the ability of mAb-EspB-B7 to inhibit the interaction between EspB and EspD, 100 or 200 nM of mAb-EspB-B7 were added to EPEC ΔespD expressing EspD-35His sample. The supernatants, containing secreted EspD-³⁵His and EspB, were collected by centrifugation (20000×g for 5 min) and were passed through a 0.45-μm-pore-size filter. Protease inhibitor solution was added to the samples (200 mM PMSF and 1 μM benzamidine), and they were incubated with Ni-NTA resin while being rotated overnight at 4° C. The samples were then loaded on gravity columns, and the flow-through was collected. The columns were washed three times with 5 mL of washing buffer (30 mM phosphate buffer pH 7.5, 500 mM NaCl, 50 mM imidazole), and proteins were eluted using elution buffer (30 mM phosphate buffer pH 7.5, 500 mM NaCl, 500 mM imidazole). Equal volumes of the supernatant and the eluate samples were precipitated with 10% (v/v) TCA for 1 hr at 4° C., centrifuged (30 mM, 16000× g, 4° C.), air dried, and dissolved in SDS-PAGE sample buffer. Supernatants and eluted samples were analyzed by SDS-PAGE and western blotting using mouse anti-His and mouse anti-EspB antibodies, to avoid detection of the human mAB-EspB-B7 antibody.

### Epitope Mapping Using Peptide Array

Peptide microarrays of 15-residues cyclic peptides, derived from the EspB sequence and containing an overlap of 11 residues, were obtained from JPT Peptide Technologies GmbH. Peptide array analysis was carried out according to the manufacturer protocols. Each microarray included three identical subarrays as technical triplicates. Full-length EspB protein was spotted on the array and used as a positive control, while bovine serum albumin (BSA) served as a negative control. The binding of mAb-EspB-B7 to the peptide array was carried out according to the manufacturer's instructions (www.jpt.com), with minor modifications. Briefly, 20 μg/mL mAb-EspB-B7 (0.1% TBST v/v) were incubated on the peptide microarray for 2 hr at room temperature. The peptide microarray slides were then washed (five times with TBST), incubated with Alexa Fluor 647-affinipure mouse anti-human IgG (Jackson ImmunoResearch) for 45 mM at room temperature, washed (five times with TBST and then five times with doubly distilled H2O), and dried. Fluorescence was detected with a GenePix 4000B scanner (Molecular Devices) at a resolution of 10 μm pixel size and analyzed by the Genepix Pro 6.0 analysis software (Molecular Devices). Signals were normalized and plotted to reflect the relative intensities of the fluorescence signals.

### Effector Translocation Activity

Translocation assays were performed as previously described. More specifically, HeLa cells (8×10⁵ cells per well) were infected for 3 hr with EPEC strains that were pre-induced for 3 hr for T3SS activity (pre-heated DMEM, statically, in a CO2 tissue culture incubator). Cells were then washed with PBS, collected, and lysed with RIPA buffer. Samples were centrifuged at 18000×g for 5 min to remove non-lysed cells, and supernatants were collected, mixed with SDS-PAGE sample buffer, and subjected to western blot analysis with anti-JNK and anti-actin antibodies (loading control). Uninfected samples and the ΔescN mutant strain-infected samples were used as negative controls. To evaluate the ability of mAb-EspB-B7 to inhibit EPEC translocation activity, 400 nM of mAb-EspB-B7 were added to a sample infected with WT EPEC.

### Nano Differential Scanning Fluorimetry (NanoDSF)

To assess the thermal stability of mAb-B7, 20 μM mAb-B7 samples were loaded into UV capillaries (NanoTemper Technologies) and analyzed using the nanoDSF Prometheus NT.48. The temperature gradient was set to 1° C./min increase between 15° C. and 95° C. The melting temperatures (Tm) that was derived from protein unfolding was presented by plotting the tryptophan fluorescence at κ=330 nm and 2=350 nm over temperature. The melting temperatures were determined by calculating the maximum of the first derivative and the peak position (at Tₘ) was determined.

### Example 1

### Anti-EspB Antibody Preparation Utilizing Phage Bio Panning

scFv phage displaying libraries are powerful and rapid tool for in-vitro isolation of antibodies fragments against any desired antigen. To isolate antibodies targeting the EspB subunit of the EPEC T3SS, a phage displaying scFv human synthetic library was used, as previously described (Kuhn, P. et al. (2016), Prot. Clin. Appl., 10: 922-948; Benhar I et al. (2004) Journal of Molecular Biology Vol 335, Issue 1, p. 177-192)). EspB-His was expressed under T3SS condition and purified from supernatant of EPEC WT that secrete the protein to the extracellular medium. The protein was loaded on Ni-beads; fraction containing the protein were collected and further purified on a size-exclusion chromatography column Following the third and fourth selection cycles, phage outputs were used from these cycles to separately infect E. coli TG-1 cultures. Phages scFv clones prepared from 190 infected single E. coli TG-1 colonies were picked randomly, were tested for high affinity and specificity to EspB by phage-ELISA. These phages expressing scFv on their surface, were individually incubated with EspB or a control antigen (BSA or TNF-alpha) and detected with anti-phage HRP conjugate antibody (FIG. 1). To evaluate the binding affinity and specificity of each isolated clone, a ratio binding signal to EspB/control-antigen was calculated. Eleven clones from the third cycle (FIG. 1A) and seven clones from the forth cycle (FIG. 1B) showing the highest ratio of binding signal were chosen for downstream applications. scFv phage displaying libraries may introduce some biases such as isolation of background binders coming from binding to solid surface materials, non-specific phage binders from ‘bald’ phages that do not present scFv on their surface, or binders to unwanted antigen in the environment such as blocking solution proteins (BSA, skim milk) and recombinant protein-tags (His). To deal with this issue the bio-panning process includes a depletion steps of background binders by alternating the bio panning against control antigens. The scFv-soluble ELISA assay helps predicting if isolated clones will maintain their affinity and specificity as full IgG molecules. Therefore, the binding affinity and specificity of the eighteen scFv clones detected by phage-ELISA were evaluated with their corresponding scFv soluble fragments that were expressed in and harvested from E. coli cells (FIG. 2A, FIG. 2B). Briefly, E. coli cells were lysed by sonication and the binding affinity of scFv clones isolated from the third bio-panning cycle were tested against EspB, and from the 4 th bio-panning cycle were tested against EspB and additional two control antigens by scFv-soluble ELISA (FIG. 2C). Clones B7, G11 and D12 from cycle 4 (FIG. 1B), showed highest binding affinity to EspB, and were further evaluated for binding specificity by scFv-soluble ELISA to EspB and two control antigens. The three clones that showed high binding specificity to EspB, were selected for further evaluation as full-length IgG molecules targeting EspB protein. Sequence analysis of the V H and V_{L} genes of the three clones revealed identical sequences, therefore only one selected clone (B7) proceeded to downstream analysis. To express a full IgG encoding the variable regions from B7 scFv clone, the V_{H} and V_{L} genes from B7 encoding phagemid were amplified using polymerase chain reaction (PCR). Mammalian expression vectors (pcDNA 3.4 heavy or pcDNA 3.4 light) encoding the heavy and light chain constant regions of IgG1 isotype and lambda light chain, respectively, were amplified to be used as scaffold for Gibson cloning of the VH and VL genes encoding B7 scFv clone. VH+VL genes were cloned into pcDNA3.4 heavy and light chain vectors, respectively, and transformed into E. coli competent cells. To produce full-length functional IgG₁ from B7 clone using pcDNA3.4-IgH/IgL vectors, mammalian cell expression system was utilized. pcDNA3.4-IgH/IgL vectors were transiently co-transfected into HEK-293 Expi™ cells that express heavy and light chains and secret them in an assembled IgG₁ form to their growth medium. Following expression and purification utilizing protein A affinity column, B7 IgG₁ binding affinity and specificity to EspB or control antigen (human TNF-alpha) was determined. FIG. 3 provides a schematic representation of the amino acid and encoding nucleic acid sequences of the V_{H} (FIG. 3A, as denoted by SEQ ID NO: 2 and SEQ ID NO: 1, respectively) and the V_{L} (FIG. 3B, as denoted by SEQ ID NO: 18 and SEQ ID NO: 17, respectively) of mAb-B7 as well as its 6 CDRs (as denoted by SEQ ID NO: 6, 10, 14 and SEQ ID NO: 22, 26, 30, respectively). The employed library was as described previously in Azriel-Rosenfeld R, et al. (Azriel-Rosenfeld R, et al. Journal of molecular biology (2004) 335(1):177-92).

### Example 2

### Affinity and Specificity of Lead mAb-B7

### mAb-B7 Binding Affinity to EspB

In order to determine the binding affinity of the lead mAb-B7 to its EspB target, indirect ELISA and Surface Plasmon Resonance (SPR) were used. As determined by ELISA (FIG. 4A) and SPR (FIG. 4B), mAb-B7 binds to EspB with the affinity of KD=17.4 nM. Additionally, high affinity of mAb-B7 to EspB was detected in human serum (FIG. 4C), which gives an indication that the antibody can bind EspB under physiological conditions. Still further, to determine whether mAb-EspB-B7 binds specifically to EspB under native conditions, the binding of mAb-EspB-B7 to WT EPEC, ΔescN (a mutant lacking a functional T3SS), ΔespB (a mutant that does not express and secrete EspB), and ΔespB+EspB-His (an espB null strain that overexpresses plasmid-encoded EspB-His), was examined. The different strains grown under T3SS-inducing conditions were separated into bacterial pellets and supernatants, which were then analyzed by SDS-PAGE and western blot using mAb-EspB-B7 in the detection phase. In agreement with a previous study, a significant secretion of EspB into the bacterial supernatant of WT EPEC was determined, however, not into the supernatants of the ΔescN or ΔespB mutants (FIG. 5A). Moreover, the inventors observed that overexpression of EspB (ΔespB+EspB−His) resulted in a higher expression of the protein within the bacteria (pellet) as well as a higher secretion into the extracellular medium (FIG. 5A).

### Example 3

### mAb-EspB-B7 Binding to EspB in the Assembled T3SS

To examine whether mAb-EspB-B7 can bind to the native protein in the assembled T3SS, flow cytometry was used. For this purpose, the bacterial strains grown under T3SS-inducing conditions were incubated first with mAb-EspB-B7 and then with a secondary antibody conjugated to a fluorophore. As expected, mAb-EspB-B7 binding was detected in WT EPEC and in the ΔespB strain overexpressing EspB, whereas no or minimal binding was detected in the ΔescN and ΔespB mutant strains (FIG. 5B).

### Example 4

### Stability of mAb-EspB-B7 Binding Under Various Physiochemical Conditions

Next, the ability of mAb-EspB-B7 to bind EspB was evaluated under various conditions by ELISA. Examination of mAb-EspB-B7 binding under different pH conditions demonstrated that the binding was essentially not altered under a wide range of pH values (5.6-7.4), with the exception of pH 4.6, at which (as expected) there was a reduction in binding capacity (FIG. 6A). FIG. 6B shows mAb-EspB-B7 binding under different temperatures. As shown by the figure, the antibody maintains binding in physiological temperatures of 37° C. and 40° C. and in room temperature. Interestingly, testing mAb-EspB-B7 across a wide range of NaCl concentrations demonstrated that only increased salt concentrations (>250 mM) affected the binding capacity of the antibody (FIG. 6C). Finally, to assess the thermal stability of mAb-EspB-B7, the melting temperature of the antibody was determined, both alone and in complex with purified EspB, by using nanoDSF. The melting temperatures of 75.4° C. and 82° C. for mAb-EspB-B7 alone and in complex with EspB, respectively (FIG. 6D), indicated high mAb stability.

### Example 5

### Non-Interference of mAb-EspB-B7 with the EspB-EspD Interaction

The EspB protein is found in a complex with another T3SS protein, called EspD, within the assembled T3SS. To confirm that the mAb-EspB-B7 epitope is exposed following EspB-EspD interaction, the ability of EspB to co-elute with EspD in the absence or the presence of mAb-EspB-B7 (100 or 200 nM), was evaluated. As observed in FIG. 7, the presence of mAb-EspB-B7 did not affect the co-elution of EspB with EspD, suggesting that mAb-EspB-B7 does not interfere with the EspB-EspD interaction. Low non-specific binding of EspB to the Ni-NTA beads was observed in the negative control (a sample that did not express EspD-³⁵Hs).

### Example 6

### mAb-EspB-B7 Epitope Mapping

To identify the exact epitope of mAb-EspB-B7 within the EspB protein, a peptide array of 78 cyclic peptides that covers the full sequence of EspB (321 residues long), was designed. Each peptide was 15 residues long, with an overlap of 11 residues between the peptides. Recombinant EspB (full-length) served as a positive control, while BSA served as a negative control. Incubation of mAb-EspB-B7 with the peptide array revealed that mAb-EspB-B7 bound mostly to two cyclic peptides within the array, namely, to peptides #49 (positions 193-207) and #50 (positions 197-211), which have the sequences TSAQKASQVAEEAAD (SEQ ID NO: 33) and KASQVAEEAADAAQE (SEQ ID NO: 35) of the EspB protein, respectively (FIG. 8A). To confirm that this epitope is indeed recognized by mAb-EspB-B7, the following peptides were synthesized: peptide #49; peptide #50; a peptide that comprises the combined sequences of peptides #49 and #50 (TSAQKASQVAEEAADAAQE) (SEQ ID NO: 37); peptide #78 (SEQ ID NO: 38), which was not detected by the mAb-EspB-B7 and was therefore suitable as a negative control; and two peptides with scrambled sequences of peptides #49 and #50. Competitive ELISA between full-length EspB and the cyclic peptides revealed that pre-incubation of peptides #49, #50 or #49+#50 (1 μg/ml) (SEQ ID NOs. 33, 35, 37, respectively), with mAb-EspB-B7 completely abolished the ability of the antibody to bind full-length EspB (FIG. 8B, 8C, 8D). To determine whether the competitive effect is derived directly from the binding of mAb-EspB-B7 to the peptides, the ability of mAb-EspB-B7 to recognize and bind these peptides was assessed. As observed in FIG. 9, mAb-EspB-B7 can bind peptides #49, #50 (SEQ ID NOs. 33, 35) and the combined peptide (#49+#50) (SEQ ID NOs. 37), while no binding was detected for the scrambled peptides or for peptide #78 (FIG. 9A, 9B, 9C). These results confirm that the main mAb-EspB-B7 epitope is the KASQVAEEAAD sequence of the EspB protein (SEQ ID NO: 49). The sequences of the peptides are presented in Table 1.

TABLE 1
peptides for epitope mapping (Sequences of the indicated peptides)

| | | SEQUENCE |
|---|---|---|
| Peptide | Sequence | ID NO: |
| Peptide #49 | NH2-CTSAQKASQVAEEAADC- | 33 |
| | CONH2 | |
| Peptide #49-sc | NH2-CSSEVQAAKADTEAQAC- | 34 |
| | CONH2 | |
| Peptide #50 | NH2-CKASQVAEEAADAAQEC- | 35 |
| | CONH2 | |
| Peptide #50-sc | NH2-CDEAVAQAAAEKEAQSC- | 36 |
| | CONH2 | |
| Peptide #49 + 50 | NH2- | 37 |
| | CTSAQKASQVAEEAADAAQEC- | |
| | CONH2 | |
| Peptide #49 + 50-sc | NH2- | 44 |
| | CAEATQEADVAEQASKASQAC- | |
| | CONH2 | |
| Peptide #78 | NH2-CTDLINRMGQAARLAGC- | 38 |
| | CONH2 | |

### Example 7

### mAb-EspB-B7 Specificity Towards EPEC EspB Homologs

EPEC cannot be used in animal model experiments since it is not pathogenic in mice. Hence, in preparation for upcoming in vivo experiments, the alignment between the sequences of EspB of EPEC and the EspB of Citrobacter rodentium (C. rodentium) was examined C. rodentium is the related murine pathogen that is commonly used in in vivo experiments of bacterial diarrheal diseases. To assess the specificity of mAb-EspB-B7 toward EspB homologs in other bacterial pathogens and its potential to be used for detection of bacteria related to other infectious diseases, bacterial cultures grown under T3SS-inducing conditions were centrifuged, and supernatants and pellets were analyzed by SDS-PAGE and western blotting using mAb-EspB-B7. The following WT bacteria and T3SS-mutant strains were cultured: EPEC; enterohemorrhagic E. coli (EHEC), which causes a more severe disease than EPEC in humans; C. rodentium, an EPEC-related mouse pathogen; and Salmonella enterica serovar Typhimurium, which utilizes two T3SSs for virulence. The strongest signal was observed for the WT EPEC supernatant; a significant, but less strong, signal was also detected for C. rodentium, and an even less strong signal, for EHEC (FIG. 10A). However, no signals were detected in the supernatants of Salmonella or any of the T3SS mutant strains. Sequence alignment of EPEC and C. rodentium EspB proteins revealed high conservation between the proteins and full conservation of the mAb-EspB-B7 epitope sequence. Sequence alignment between EPEC and EHEC EspB proteins revealed 80% similarity at the mAb-EspB-B7 epitope region (FIG. 10B). Thus, these results showed that the mAb-B7 antibody of the present disclosure recognizes the C. rodentium protein and its animal model is likely to be suitable for the specified needs.

### Example 8

### Effect of mAb-EspB-B7 on Bacterial-Host Cell Interaction

To examine the ability of mAb-EspB-B7 to directly interfere with the bacterial infection of host cells, the translocation activity of WT EPEC was examined in the presence or absence of mAb-EspB-B7. For that purpose, HeLa cells were infected with EPEC strains (WT and ΔescN) and the cleavage pattern of JNK was examined, a host protein that is cleaved by a translocated EPEC effector known as NleD (FIG. 11A). WT EPEC caused extensive degradation of JNK, relative to the uninfected sample and the samples infected with the ΔescN mutant strain (FIG. 11B). The inventors found that, HeLa cells infected by WT EPEC in the presence of a high concentration of mAb-EspB-B7 (400 nM) showed translocation activity similar to that of WT EPEC infection with no addition of antibody. These results suggest that while mAb-EspB-B7 was capable of binding EspB with high affinity and specificity, this binding did not interfere with the protein function and did not inhibit the T3SS translocation activity in an ex vivo model.

### Example 9

### Selection of Animal Infection Model

The Citrobacter rodentium, the gram-negative murine-specific pathogen closely related to the human pathogens EPEC and EHEC is selected.

Bacteria from frozen culture are inoculated onto agar plate. Bacteria are grown overnight. Oral gavage inoculation of groups of three mice is performed at the following dose-range: 1.6×10⁷, 4×10⁷, 1.0 ×10⁸, 2.5×10⁸ cfu. Each group of three mice is sacrificed on days 5, 7 or 9. The following parameters are examined for evaluation of pathogenicity: measure of colonic epithelial barrier permeability, measure of bacterial load in cecum and colon and assessment of histology or of immunofluorescence staining of infected colon tissues. Then, the inoculum level and day of sacrifice that is on linear part of dose-response curve for signals in permeability, bacterial load, and histology/staining studies are selected. In addition, 2 or 3 parameters are selected for evaluation of pathogenicity that are consistent in each group.

### Example 10

### Inhibition of Pathogenesis in Animal Models

Injection of mAb-B7 to mice is tested. The dose-range of mAb at 3-4 dosage levels is based on data from ex-vivo inhibition experiments. The mAb or PBS are injected IP to groups of three mice 24 hours before administration of C. rodentium by oral gavage, then twice a day. The mice are sacrificed mice on the selected sacrifice day (5 or 7 or 9).

The efficacy is then evaluated. The parameters employed for evaluation of efficacy are as selected from above calibration study. Effective mAb dose are selected for synergy studies that is on plateau of dose-response curve. For evaluation of efficacy, 1 or 2 parameters are selected based on effectiveness and reproducibility.

### Example 11

### Synergy with Standard Antibiotics

Injection of antibiotic together with mAb-B7 is tested in mice. The in-vitro antibiotic sensitivity of strain is evaluated with common antibiotics and two antibiotics are selected. Each antibiotic are tested at 3-4 dosage levels based on data from in-vitro sensitivity experiment. Antibiotics are injected IP to groups of three mice simultaneous with oral gavage of C. rodentium, then daily thereafter. The mice are sacrificed on the selected sacrifice day.

The efficacy of the antibiotic is then evaluated by employing parameters for evaluation of pathogenicity as selected from the mAb studies. The antibiotic level that is on linear part of dose-response curve for signals in permeability, bacterial load, and histology/staining studies is selected. Next, co-administration of mAb and antibiotic is performed in mice. The mAb at selected dose level or PBS are injected IP to groups of three mice on day 1 and then twice a day. Antibiotics at selected dose level or PBS are injected to groups of three mice on day 0, then daily thereafter. Oral gavage of C. rodentium at selected cfu level or PBS is performed immediately after injection of antibiotic on day 0. The mice are sacrificed on the selected sacrifice day. The efficacy of the coadministration of mAb and antibiotic is then evaluated by employing parameters for evaluation of pathogenicity as selected from the mAb studies. eatment and diagnosis of MDR pathogenic bacterial infections.

1. An antibody or any antigen-binding fragment thereof,
wherein said antibody comprises at least one heavy chain complementarity determining region (CDRH) 1 comprising the amino acid sequence GFTFSHYA, as denoted by SEQ ID NO: 6, CDRH2 comprising the amino acid sequence INSNGDST, as denoted by SEQ ID NO: 10, CDRH3 comprising the amino acid sequence ARDRRAGYFDYW, as denoted by SEQ ID NO: 14, and at least one light chain complementarity determining region (CDRL) 1 comprising the amino acid sequence RDNIGKNY as denoted by SEQ ID NO: 22, CDRL2 comprising the amino acid sequence RNN as denoted by SEQ ID NO: 26, and CDRL3 comprising the amino acid sequence SAWDTSLNA as denoted by SEQ ID NO: 30, or any derivative, variant and biosimilar thereof.

2. The antibody according to claim 1, wherein said antibody specifically recognizes and binds at least one component of the Type III Secretion System (T3SS) of at least one bacterium.

3. The antibody according to claim 1, wherein at least one of:
(a) said bacterium is at least one Multiple Drug Resistant (MDR) bacterium; and
(b) said MDR bacterium is at least one of Enteropathogenic Escherichia coli (EPEC) and Enterohemorrhagic Escherichia coli (EHEC).

4. (canceled)

5. The antibody according to claim 1, wherein said antibody binds the Escherichia coli secreted protein B (EspB) expressed by said bacterium.

6-7. (canceled)

8. A pharmaceutical composition comprising as an active ingredient the isolated monoclonal antibody according to claim 1 or any antigen-binding fragment thereof or any matrix, nano- or micro-particles thereof, or any derivative, variant and biosimilar thereof, said composition optionally further comprises at least one of a pharmaceutically acceptable carrier, excipient and diluent.

9. (canceled)

10. A method of treating, preventing, ameliorating, reducing or delaying the onset of an infection by at least one bacterium expressing at least one T3SS in a subject in need thereof, comprising the step of administering to said subject a therapeutically effective amount of at least one antibody or any antigen-binding fragment thereof, any matrix, nano- or micro-particles thereof, or of any composition comprising said antibody, wherein said antibody comprises at last one heavy chain CDRH1 comprising the amino acid sequence GFTFSHYA, as denoted by SEQ ID NO: 6, CDRH2 comprising the amino acid sequence INSNGDST, as denoted by SEQ ID NO: 10, CDRH3 comprising the amino acid sequence ARDRRAGYFDYW, as denoted by SEQ ID NO: 14, and at least one light chain CDRL1 comprising the amino acid sequence RDNIGKNY as denoted by SEQ ID NO: 22, CDRL2 comprising the amino acid sequence RNN as denoted by SEQ ID NO: 26, and CDRL3 comprising the amino acid sequence SAWDTSLNA as denoted by SEQ ID NO: 30, or any derivative, variant and biosimilar thereof.

11. The method according to claim 10, wherein said antibody specifically recognizes and binds at least one component of the T3SS of at least one bacterium.

12. The method according to claim 11, wherein said bacterium is at least one MDR bacterium, optionally, wherein said MDR bacterium is at least one of EPEC and EHEC.

13. The method according to claim 11, wherein said antibody binds the EspB expressed by said bacterium.

14. The method according to claim 10, wherein said antibody modulates the immune system of said subject, optionally, wherein said antibody induce at least one of a humoral response and serological memory in said subject.

15. The method according to claim 10, wherein said infection is associated with at least one of transient enteritis or colitis, cholecystitis, bacteremia, cholangitis, urinary tract infection (UTI), traveler's diarrhea, neonatal meningitis and pneumonia, or any conditions, symptoms or effects associated therewith.

16. The method according to claim 10, wherein said method further comprising the step of administering to said subject a therapeutically effect amount of at least one therapeutic agent, optionally, said agent is at least one anti-bacterial agent.

17. A method of treating, preventing, ameliorating, reducing or delaying the onset of an infection by at least one bacterium expressing at least one T3SS comprising the step of administering to a subject treated with at least one anti-bacterial agent, a therapeutically effective amount of the antibody according to claim 1, or any antigen-binding fragment thereof, any matrix, nano- or micro-particles thereof, or any composition comprising said antibody, or any derivative, variant and biosimilar thereof, optionally wherein at least one of:
(a) said MDR bacterium is at least one of Enteropathogenic Escherichia coli (EPEC) and Enterohemorrhagic Escherichia coli (EHEC);
(b) said infection is associated with at least one of transient enteritis or colitis, cholecystitis, bacteremia, cholangitis, UTI, traveler's diarrhea, neonatal meningitis and pneumonia, or any condition, symptoms or effects associated therewith; and said antibody binds the EspB expressed by said bacterium.

18-19. (canceled)

20. The method according to claim 17, wherein said antibacterial agent is at least one antibiotic agent or any combinations thereof.

21. (canceled)

22. A kit comprising:
a) at least one antibody according to claim 1, or any antigen-binding fragment thereof or any matrix, nano- or micro-particles thereof, or any derivative, variant and biosimilar thereof; and
b) at least one therapeutic agent.

23-26. (canceled)

27. A diagnostic method for detecting at least one bacterium expressing a T3SS in at least one biological sample, said method comprising:
a) contacting said at least one biological sample with at least one antibody or any antigen-binding fragment thereof, or any matrix, nano- or micro-particles thereof, said antibody comprising at least one heavy chain CDRH1 comprising the amino acid sequence GFTFSHYA, as denoted by SEQ ID NO: 6, CDRH2 comprising the amino acid sequence INSNGDST, as denoted by SEQ ID NO: 10, CDRH3 comprising the amino acid sequence ARDRRAGYFDYW, as denoted by SEQ ID NO: 14, and at least one light chain CDRL1 comprising the amino acid sequence RDNIGKNY as denoted by SEQ ID NO: 22, CDRL2 comprising the amino acid sequence RNN as denoted by SEQ ID NO: 26, and CDRL3 comprising the amino acid sequence SAWDTSLNA as denoted by SEQ ID NO: 30, or any derivative, variant and biosimilar thereof, wherein said antibody or any antigen binding fragment thereof is directly or indirectly associated with at least one detectable moiety; and
b) determining the presence of at least one bacterium expressing a T3SS in said biological sample, if said detectable moiety is detected.

28. (canceled)

29. The method according to claim 27, wherein at least one of:
(a) said bacterium is at least one MDR bacterium; and
(b) said MDR bacterium is at least one of EPEC and EHEC.

30. (canceled)

31. The method according to claim 27, for detecting an infection of a mammalian subject by at least one T3SS expressing bacteria.

32. A diagnostic kit comprising:
a) at least one antibody according to claim 1, or any antigen-binding fragment thereof, or any matrix, nano- or micro-particles thereof, or any derivative, variant and biosimilar thereof, wherein said antibody is directly or indirectly associated with at least one detectable moiety; said kit optionally further comprises at least one of:
b) means for detecting the presence of said at least one detectable moiety;
c) at least one control sample and/or control standard value;
d) instructions for use of said kit.

33-38. (canceled)

39. A method for modulating the immune system of a subject infected by at least one T3SS expressing bacteria, comprising the step of administering to said subject an effective amount of at least one monoclonal antibody according to claim 1, or any antigen-binding fragment thereof, or any matrix, nano- or micro-particles thereof, or any derivative, variant and biosimilar thereof, wherein, optionally, said subject suffers from at least one of transient enteritis or colitis, cholecystitis, bacteremia, cholangitis, urinary tract infection (UTI), traveler's diarrhea, neonatal meningitis and pneumonia, or any conditions, symptoms or effects associated therewith.

40. (canceled)


SEQUENCE LISTING (49 sequences)

SEQ ID NO: 1 - DNA, 354 bp, Artificial Sequence
  B7-VH Full Sequence
gaggtgcagctgttggagtctgggggaggcttggtacagcctggggggtccctgagactctcctgtgcagcctctggattcacctttagccactatgccatgaactgggtccgccaggctccagggaaggggctggagtgggtttcagccattaatagtaatggtgatagcacctactatccagacactgtgaaggaccgattcaccatctccagagacaattccaagaacacgctgtatctgcaaatgaacagcctgagagccgaggacacggccgtgtattactgtgcgagagatcggcgagcagggtactttgactactggggccagggcaccctggtcacagtctcctca

SEQ ID NO: 2 - protein, 118 aa, Artificial Sequence
  B7-VH  Full Sequence
EVQLLESGGGLVQPGGSLRLSCAASGFTFSHYAMNWVRQAPGKGLEWVSAINSNGDSTYYPDTVKDRFTISRDNSKNTLYLQMNSLRAEDTAVYYCARDRRAGYFDYWGQGTLVTVSS

SEQ ID NO: 3 - DNA, 75 bp, Artificial Sequence
  B7-VH FR1
gaggtgcagctgttggagtctgggggaggcttggtacagcctggggggtccctgagactctcctgtgcagcctct

SEQ ID NO: 4 - protein, 25 aa, Artificial Sequence
  B7-VH FR1
EVQLLESGGGLVQPGGSLRLSCAAS

SEQ ID NO: 5 - DNA, 24 bp, Artificial Sequence
  B7-VH CDRH1
ggattcacctttagccactatgcc

SEQ ID NO: 6 - protein, 8 aa, Artificial Sequence
  B7-VH CDRH1
GFTFSHYA

SEQ ID NO: 7 - DNA, 51 bp, Artificial Sequence
  B7-VH FR2
atgaactgggtccgccaggctccagggaaggggctggagtgggtttcagcc

SEQ ID NO: 8 - protein, 17 aa, Artificial Sequence
  B7-VH FR2
MNWVRQAPGKGLEWVSA

SEQ ID NO: 9 - DNA, 24 bp, Artificial Sequence
  B7-VH CDRH2
attaatagtaatggtgatagcacc

SEQ ID NO: 10 - protein, 8 aa, Artificial Sequence
  B7-VH CDRH2
INSNGDST

SEQ ID NO: 11 - DNA, 114 bp, Artificial Sequence
  B7-VH FR3
tactatccagacactgtgaaggaccgattcaccatctccagagacaattccaagaacacgctgtatctgcaaatgaacagcctgagagccgaggacacggccgtgtattactgt

SEQ ID NO: 12 - protein, 38 aa, Artificial Sequence
  B7-VH FR3
YYPDTVKDRFTISRDNSKNTLYLQMNSLRAEDTAVYYC

SEQ ID NO: 13 - DNA, 36 bp, Artificial Sequence
  B7-VH CDRH3
gcgagagatcggcgagcagggtactttgactactgg

SEQ ID NO: 14 - protein, 12 aa, Artificial Sequence
  B7-VH CDRH3
ARDRRAGYFDYW

SEQ ID NO: 15 - DNA, 30 bp, Artificial Sequence
  B7-VH J Region
ggccagggcaccctggtcacagtctcctca

SEQ ID NO: 16 - protein, 10 aa, Artificial Sequence
  B7-VH J Region
GQGTLVTVSS

SEQ ID NO: 17 - DNA, 330 bp, Artificial Sequence
  B7- VL (lambda) Full Sequence
gatatcgtgctgactcagccaccctcagcgtctgggacccccgggcagagggtcaccatctcctgctctggaagcagggacaacatcgggaagaattatgtgtcctggtaccagcagcttccaggaacagcccccaaactcctcatttataggaataatcagcggccctcaggggtccctgaccggttctctggctccaagtctggcacctcagcctccctggccatcagtgggctgcggtccgaggatgaggctgattattactgctcagcgtgggacaccagcctcaatgcttatgtcttcgggcctggcacccagctcaccgtcctc

SEQ ID NO: 18 - protein, 110 aa, Artificial Sequence
  B7- VL (lambda) Full Sequence
DIVLTQPPSASGTPGQRVTISCSGSRDNIGKNYVSWYQQLPGTAPKLLIYRNNQRPSGVPDRFSGSKSGTSASLAISGLRSEDEADYYCSAWDTSLNAYVFGPGTQLTVL

SEQ ID NO: 19 - DNA, 75 bp, Artificial Sequence
  B7- VL FR1
gatatcgtgctgactcagccaccctcagcgtctgggacccccgggcagagggtcaccatctcctgctctggaagc

SEQ ID NO: 20 - protein, 25 aa, Artificial Sequence
  B7- VL FR1
DIVLTQPPSASGTPGQRVTISCSGS

SEQ ID NO: 21 - DNA, 24 bp, Artificial Sequence
  B7- VL CDRL1
agggacaacatcgggaagaattat

SEQ ID NO: 22 - protein, 8 aa, Artificial Sequence
  B7- VL CDRL1
RDNIGKNY

SEQ ID NO: 23 - DNA, 51 bp, Artificial Sequence
  B7- VL FR2
gtgtcctggtaccagcagcttccaggaacagcccccaaactcctcatttat

SEQ ID NO: 24 - protein, 17 aa, Artificial Sequence
  B7- VL FR2
VSWYQQLPGTAPKLLIY

SEQ ID NO: 25 - DNA, 10 bp, Artificial Sequence
  B7- VL CDRL2
  misc_feature (10)..(10): n is null
aggaataatn

SEQ ID NO: 26 - protein, 4 aa, Artificial Sequence
  B7- VL CDRL2
  MISC_FEATURE (4)..(4): Xaa is null
RNNX

SEQ ID NO: 27 - DNA, 108 bp, Artificial Sequence
  B7- VL FR3
cagcggccctcaggggtccctgaccggttctctggctccaagtctggcacctcagcctccctggccatcagtgggctgcggtccgaggatgaggctgattattactgc

SEQ ID NO: 28 - protein, 36 aa, Artificial Sequence
  B7- VL FR3
QRPSGVPDRFSGSKSGTSASLAISGLRSEDEADYYC

SEQ ID NO: 29 - DNA, 27 bp, Artificial Sequence
  B7- VL CDRL3
tcagcgtgggacaccagcctcaatgct

SEQ ID NO: 30 - protein, 9 aa, Artificial Sequence
  B7- VL CDRL3
SAWDTSLNA

SEQ ID NO: 31 - DNA, 36 bp, Artificial Sequence
  B7- VL J Region
tatgtcttcgggcctggcacccagctcaccgtcctc

SEQ ID NO: 32 - protein, 12 aa, Artificial Sequence
  B7- VL J Region
YVFGPGTQLTVL

SEQ ID NO: 33 - protein, 17 aa, Artificial Sequence
  PEPTIDE #49
CTSAQKASQVAEEAADC

SEQ ID NO: 34 - protein, 17 aa, Artificial Sequence
  PEPTIDE #49-SC
CSSEVQAAKADTEAQAC

SEQ ID NO: 35 - protein, 17 aa, Artificial Sequence
  PEPTIDE #50
CKASQVAEEAADAAQEC

SEQ ID NO: 36 - protein, 17 aa, Artificial Sequence
  PEPTIDE #50-SC
CDEAVAQAAAEKEAQSC

SEQ ID NO: 37 - protein, 21 aa, Artificial Sequence
  PEPTIDE #49+50
CTSAQKASQVAEEAADAAQEC

SEQ ID NO: 38 - protein, 17 aa, Artificial Sequence
  PEPTIDE #78
CTDLINRMGQAARLAGC

SEQ ID NO: 39 - protein, 18 aa, Artificial Sequence
  EPITOPE OF B7
TSAQKASQVAEEAADAAQ

SEQ ID NO: 40 - protein, 321 aa, Escherichia coli
  MISC_FEATURE: EspB, EPEC Accession number WP_001091991.1
MNTIDNNNAAIAVNSVLSSTTDSTSSTTTSTSSISSSLLTDGRVDISKLLLEVQKLLREMVTTLQDYLQKQLAQSYDIQKAVFESQNKAIDEKKAGATAALIGGAISSVLGILGSFAAINSATKGASDVAQQAASTSAKSIGTVSEASTKALAKASEGIADAADDAAGAMQQTIATAAKAASRTSGITDDVATSAQKASQVAEEAADAAQELAQKAGLLSRFTAAAGRISGSTPFIVVTSLAEGTKTLPTTISESVKSNHDINEQRAKSVENLQASNLDTYKQDVRRAQDDISSRLRDMTTTARDLTDLINRMGQAARLAG

SEQ ID NO: 41 - DNA, 945 bp, Escherichia coli
  misc_feature: EspB, EPEC Accession number AAB69980.1
atgaatactattgattatactaatcaagtaatgacggttaattctgtttcggagaatactaccggctctaatgcaattaccgcatctgctattaattcatctttgcttaccgatggtaaggtcgatgtttctaaactgatgctggaaattcaaaaactcctgggcaagatggtgcgtatattgcaggattaccaacagcaacagttgtcgcagagctatcagatccaactggccgtttttgagagccagaataaagccattgatgaaaaaaaggccgctgcaacagccgctctggttggtggggctatttcatcagtattggggatcttaggctcttttgcagcaattaacagtgctacgaaaggcgcgagtgatattgctcaaaaaaccgcctctacatcttctaaggctattgatgcggcttctgatactgcgactaaaacgttgactaaggcaacggaaagcgttgctgatgctgttgaagatgcatccagcgtgatgcagcaagcgatgactacagcaacgagagcggccagccgtacatccgacgttgctgatgacattgccgattctgctcagagagcttctcagctggctgaaaacgctgcagatgccgctcagaaggcaagtcgggcaagccgctttatggctgcagtagataagattactggctctacaccatttattgccgttaccagtcttgccgaaggcacgaagacattgccaacaacggtatctgaatcagtcaaatctaaccatgagattagcgaacagcgttataagtctgtggagaacttccagcagggtaatttggatctgtataagcaagaagttcgcagagcgcaggatgatatcgctagccgtctgcgtgatatgacaacagccgctcgcgatctcactgatcttcagaatcgtatgggtcaatcggttcgcttagctgggtaa

SEQ ID NO: 42 - protein, 380 aa, Escherichia coli
  MISC_FEATURE: EspD, EPEC Accession number WP_000935767.1
MLNVNSDIQSMRSGASAATATSGINQPEVTSALDLQLVKSTAPSASWTESTALPTPPAGHSLVTPSAAEDVLSKLFGGISGEVRSRTEGTEPQRSTQNASSGYPYLSQVNNVDPQAMMMMVTLLSLDASAQKVASMKNSNEIYTDGQNKALDNKTLEFKKQLEEQQKAEEKAQKSKIVGQVFGWLGVAVTAIAAIFNPALWAVVAVSATAMALQTAVDVMGDKAPQALKTAAQVFGGVSIAASILTAGIGGVSSLMSKVGDVANKVGSNIVKTVTKLADVFVENVVSKVAATANGFTTSARSIGTTVLNNDSAQYSILAQLSAYAVQNLTRQSENLGESAKVELDKAAAELRNQASYLQNASQLISDSARVNSRIVSGRV

SEQ ID NO: 43 - DNA, 1000 bp, Escherichia coli
  misc_feature: EspD, EPEC Accession number CAI43861.1
atgctcaatgtaaatagcgatatccagtcgatgaggtctggggccagcgctgctacggctacttcaggtattaatcagcctgaagtgacttccgcgctggatttacaactggttaaatccacggccccatcagcaagttggacagaaagtaccgctttaccaacgccaccggcaggccactcattagtgacgccctctgctgctgaggatgtcctgagtaaactgtttggtggtattagtggtgaagttaggagtcgaacagagggaaccgaaccacagcgcagcactcagaatgcaagctctggctatccttatctttctcaggtgaataacgttgaccctcaggccatgatgatgatggtcaccctgttatccctggatgcttcagcgcagaaagttgcgagtatgaaaaactccaacgagatctacacggatgggcaaaataaggcgctggataataaaacgctggagtttaaaaagcagctcgaagaacaacagaaagccgaagagaaagcacaaaaaagtaaaattgttggtcaggtctttggttggttgggcgtcgcggtaactgctattgcagctatttttaacccggcactctgggctgttgttgccgttagcgcaacagcaatggcactgcaaactgcagttgatgtaatgggcgataaggccccgcaggcgttaaagacagcagcgcaagtctttggcggagtatctattgctgcaagtattctgacagccggcattggcggggtgtcttcacttatgtctaaagttggcgatgtcgctaacaaagttggttcaaacatcgtgaaaacagttactaaactggccgatgtatttgttgagaacgttgtttcaaaagttgcggcaacggcaaatggatttacgacctctgcacgttctattgggacaactgtgctgaataatgattctgcgcaatacagcatattggctcagttatctgcttacgcggtgcaaaacttaactcgacagagtg

SEQ ID NO: 44 - protein, 21 aa, Artificial Sequence
  PEPTIDE 49-50 SC
CAEATQEADVAEQASKASQAC

SEQ ID NO: 45 - protein, 312 aa, Escherichia coli
  MISC_FEATURE: EHEC-EspB
MNTIDNTQVTMVNSASESTTGASSAVAASALSIDSSLLTDGKVDICKLMLEIQKLLGKMVTLLQDYQQKQLAQSYQIQQAVFESQNKAIEEKKAAATAALVGGIISSALGILGSFAAMNNAAKGAGEIAEKASSASSKAAGAASEVANKALVKATESVADVAEEASSAMQKAMATTTKAASRASGVADDVAKASDFAEDLADAAEKTSRINKLLNSVDKLTNTTAFVAVTSLAEGTKTLPTTISESVKSTHEVNEQRAKSLENFQQGNLELYKQDVRRTQDDITTRLRDITSAVRDLLEVQNRMGQSGRLAG

SEQ ID NO: 46 - protein, 321 aa, Citrobacter rodentium
  MISC_FEATURE: C. rodentium (CR)-EspB
MNTIDYNNAAIAVNPVVSSTTDSASSTTTLTSSSSSSLLTAGKVDISKLLLEVQKLMREMVTTLQDYLQRQLAQSYNIQQAIFQSQNKAIEEKKAGATAALIGGAISSVLGILGSFAAINSATKGASDVVQKATSASAKSVNTASEVSTKALVKASESIADAADETVGTLQQGIAAATKAANRTSGIADDVTTSAQKASQVAEEAADAAQDLTRKANLLSRFTAAAGRISGSTAFVVVTSLAEGTKTLPTTISESVKSNHDINEQRAKSVENLQASNLETYKQDVRRSQDDIASRLRDMTATARDLNDLINRMGQSARLAG

SEQ ID NO: 47 - protein, 409 aa, Salmonella
  MISC_FEATURE: Salmonella-EspB A
MLISNVGINPAAYLNNHSVENSSQTASQSVSAKDILNSIGISSSKVSDLGLSPTLSAPAPGVLTQTPGTITSFLKASIQNTDMNQDLNALANNVTTKANEVVQTQLREQQAEVGKFFDISGMSSSAVALLAAANTLMLTLNQADSKLSGKLSLVSFDAAKTTASSMMREGMNALSGSISQSALQLGITGVGAKLEYKGLQNERGALKHNAAKIDKLTTESHSIKNVLNGQNSVKLGAEGVDSLKSLNMKKTGTDATKNLNDATLKSNAGTSATESLGIKDSNKQISPEHQAILSKRLESVESDIRLEQNTMDMTRIDARKMQMTGDLIMKNSVTVGGIAGASGQYAATQERSEQQISQVNNRVASTASDEARESSRKSTSLIQEMLKTMESINQSKASALAAIAGNIRA

SEQ ID NO: 48 - protein, 27 aa, Artificial Sequence
  peptide #9-12
SISSSLLTDGRVDISKLLLEVQKLLRE

SEQ ID NO: 49 - protein, 11 aa, Artificial Sequence
  Epitope peptide
KASQVAEEAAD